(19) United States
(12) Patent Application Publication
(10) Pub. No.: US 2014/0120058 A1
(43) Pub. Date: May 1, 2014

(54) POLYMER CONJUGATED PROSTAGLANDIN ANALOGUES

(71) Applicants: Michael Shane O'Shea, Mulgrave (AU); Florian Hans Maximillian Graichen, Antwerpen (BE); Russell John Tait, Balwyn (AU); Andrew Craig Donohue, Bentleigh East (AU); Sarah Man Yee Ng, Ascot Vale (AU); Asha Marina D'Souza, Carnegie (AU)

(72) Inventors: Michael Shane O'Shea, Mulgrave (AU); Florian Hans Maximillian Graichen, Antwerpen (BE); Russell John Tait, Balwyn (AU); Andrew Craig Donohue, Bentleigh East (AU); Sarah Man Yee Ng, Ascot Vale (AU); Asha Marina D'Souza, Carnegie (AU)

(73) Assignees: POLYACTIVA PTY LTD; COMMONWEALTH SCIENTIFIC AND INDUSTRIAL RESEARCH ORGANISATION; THE BIONICS INSTITUTE OF AUSTRALIA

(21) Appl. No.: 14/111,408

(22) PCT Filed: Apr. 12, 2012
(86) PCT No.: PCT/AU2012/000376

§ 371 Date: Dec. 23, 2013

Related U.S. Application Data
(60) Provisional application No. 61/474,598, filed on Apr. 12, 2011.

(51) Int. Cl. A61K 47/48

(52) U.S. Cl. CPC A61K 47/48192
USPC 424/78.37; 528/75; 560/55; 560/186

(57) ABSTRACT

The present invention relates in general to polymer-drug conjugates. In particular, the invention relates to polymer-drug conjugates wherein the conjugated drugs are selected from prostaglandins and substituted prostaglandins, to a method of delivering such prostaglandin drugs to a subject, to a sustained drug delivery system comprising the polymer-drug conjugates, to a method of preparing the polymer-drug conjugates, and to an implant comprising the polymer-drug conjugates. The polymer-drug conjugates may be useful for delivering prostaglandins and substituted prostaglandins for the treatment of glaucoma.

### FIELD OF THE INVENTION

The present invention relates in general to polymer-drug conjugates. In particular, the invention relates to polymer-drug conjugates wherein the conjugated drugs are selected from prostaglandins and substituted prostaglandins, to a method of delivering such drugs to a subject, to a sustained drug delivery system comprising the polymer-drug conjugates, to a method of preparing the polymer-drug conjugates, and to an implant comprising the polymer-drug conjugates.

### BACKGROUND OF THE INVENTION

The targeted and controlled delivery of drugs is an area of considerable current interest. The site-specific delivery of a drug to a subject is a highly desirable feature for the treatment of many different conditions. Implantation of a device comprising a drug(s) in the body of a subject (human or animal) can be desirable to improve the efficacy and safety of the drug(s).

Certain sites in a subject may require sophisticated delivery devices to overcome barriers for effective drug delivery. For example, some sites have a limited volume for administration of a device (e.g. the eye) and require a device that has a high dose loading to ensure the device volume is kept to a minimum. Despite the limited volume it is desirable to be able to deliver the drug to the site continuously and in a controlled manner over an extended period of time.

Furthermore, such devices ideally should have material properties that ensure the subject does not experience any discomfort after the implant is placed.

One mode of delivering a drug to a subject involves the use of a polymer to carry/retain the drug to/at a specific location.

An example of such a polymer/drug delivery system utilises an admixture of a polymer with a drug, wherein the drug is blended within the polymer matrix. However, such mere admixtures generally result in poor control over the release of the drug, with a well known “burst effect” immediately after administration and a significant change in the physical properties of the admixture as the drug is released (Sjoquist, B.; Basu, S.; Byding, P.; Bergh, K.; Stjemschantz, J. Drug Metab. Dispos. 1998, 26, 745.). In addition, such admixtures have limited dose loading capacity resulting in a prohibitively large device for convenient administration to some sites in a subject.

A further example of a polymer/drug delivery system is based on the polymerisation of a drug(s) with other monomers (or itself) so as to incorporate the drug as part of the backbone polymer chain. Such a system is described by Uhlrich in U.S. Pat. No. 6,613,807, WO2008/128193, WO94/04593 and U.S. Pat. No. 7,122,615. However, such “polymerised” drugs also generally result in inefficient release of the drug as the release of the drug occurs via inactive intermediates. Such intermediates can complicate regulatory approval, which may require the safety of the intermediates to be demonstrated. Furthermore, the resulting polymer material generally has quite restricted physical properties.

Still a further example of a polymer/drug delivery system utilises a drug covalently bound to a polymer so as to form a so called polymer-drug conjugate. Examples of such polymer-drug conjugates have been reviewed in Nature Reviews: Drug Discovery 2003:2, 347-360. Such polymer-drug conjugates are typically formed by covalently attaching a drug to a preformed polymer backbone. However, the synthesis of such covalently bound systems can be problematic. In particular, steric and thermodynamic constraints can affect the amount of drug that can be covalently attached, and also impact on the distribution of the drug along the polymer backbone, which in turn can reduce control over the release of the drug. Furthermore, there is limited scope to modify the physical properties of the resulting polymer-drug conjugate material so that it can be modified to aid comfort after administration.

Substituted prostaglandins are used to treat glaucoma. They are presently formulated as eye drops, which if administered conscientiously to the affected eye will lower intraocular pressure, which in turn slows progression of the disease. Unfortunately, because glaucoma is an asymptomatic disease many patients do not use their drops conscientiously, compromising therapy. A recent study by Friedman et al. (Friedman D. S., Quigley H. A., Gelb L., Tan J., Margolis J., Shah S, N., Kim E. E., Zimmerman T., Hahn S. R. IOVS 2007:48, 5052-5057) showed that adherence to glaucoma treatment options is poor with only 59% of patients in possession of an ocular hypotensive agent at 12 months, and only 10% of patients used such medication continuously. Patient compliance in glaucoma therapy is therefore an issue.

An opportunity therefore remains to develop new polymer/drug delivery systems which address or ameliorate one or more disadvantages or shortcomings associated with existing systems and/or their method of manufacture, or to at least provide a useful alternative to such systems and their method of manufacture.

### SUMMARY OF THE INVENTION

In one aspect, the present invention provides a polymer-drug conjugate comprising a polymer backbone and a prostaglandin or substituted prostaglandin conjugated to the polymer backbone via an ester, anhydride or carbonate linking group.

In accordance with one aspect of the invention, the prostaglandin or substituted prostaglandin is linked at a position selected from the 1, 9, 11 and 15 position of the prostaglandin or substituted prostaglandin. In embodiments of the invention, the prostaglandin or substituted prostaglandin is linked via an ester linking group at a position selected from the 1, 9, 11 and 15 position of the prostaglandin or substituted prostaglandin.

In some embodiments, the polymer-drug conjugate comprises a prostaglandin drug of formula (XX):

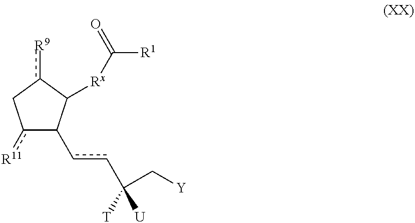

wherein:
   R^{x} is a straight chain aliphatic of six carbon atoms optionally comprising one or two substituents selected from the group consisting of oxo (═O) and hydroxy;
   [symbol] represents a double or single bond;
   T and U are selected from the group consisting of where T and U together form oxo (═O), where T and U are each halo, and where T is R¹⁵ and U is hydrogen;
   Y is optionally substituted C4 to C10 hydrocarbyl or optionally substituted C4 to C10 hydrocarbyloxy; and
   one of R¹, R⁹, R¹¹ and R¹⁵ is linked to the polymer backbone and wherein:
   R⁹, R¹¹ and R¹⁵ when linked to the polymer backbone are the alcohol residue of an ester or carbonate linking group and R¹⁵ when linked to the polymer backbone forms the acid residue of an ester or anhydride linking group; and
   R¹ when not linked to the backbone is selected from the group consisting of —OH, —O(C₁₋₆alkyl), and —NR^{a}R^{b} where R^{a} and R^{b} are each independently selected from the group consisting of H and C₁₋₆ alkyl;
   R⁹ and R¹¹ when not linked to the polymer backbone are both hydroxy or one is hydroxy and one is oxo and where one of R⁹ and R¹¹ is linked to the backbone, the other is hydroxy or oxo; and
   when R¹⁵ is not linked to the backbone then T is hydroxy and U is hydrogen, or T and U are each fluoro, or T and U together form oxo.

In one form, the polymer-drug conjugate comprises a plurality of prostaglandin drugs of formula (XXi):

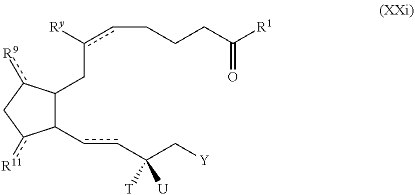

In one aspect, the present invention provides a polymer-drug conjugate comprising as part of its polymer backbone a moiety of general formula (I):

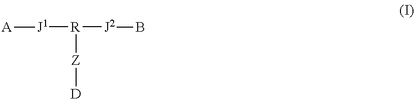

where:
   A and B, which may be the same or different, represent the remainder of the polymer backbone and are (i) attached to the -J¹R(ZD)-J²- moiety as shown in formula (I) via a bioerodible moiety, and (ii) each formed from monomeric units that are coupled via bioerodible moieties;
   J¹ and J² are independently selected from the group consisting of oxygen, C(O), and NR^{a} where R^{a} is hydrogen or C₁ to C₆ alkyl;
   R is an optionally substituted hydrocarbon;
   Z is a linking group;
   D is a prostaglandin drug of formula (XX); and
   D and Z together form an ester, anhydride or carbonate linking group.

In some embodiments, the polymer-drug conjugates in accordance with the invention comprise conjugated drugs selected from prostaglandin drugs of general formulae (XX) and (XXi). Such drugs may find use in treating hypertension, glaucoma, essential tremor, tachyarrythmias and treatment of angina and in prevention of migraines and headaches. The drugs are believed to be particularly useful in the treatment of glaucoma and hypertension.

In some embodiments of a polymer-drug conjugates in of the invention, the polymer backbone is a polyurethane, polyester, polyether, or a combination thereof, or a copolymer thereof. In some embodiments, the polymer-drug conjugate may be bioerodible.

In one form, the present invention provides a polymer-drug conjugate comprising as part of its polymer backbone a moiety of general formula (Ic):

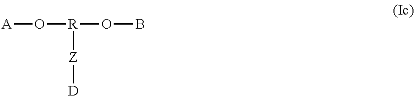

where:
   A and B, which may be the same or different, represent the remainder of the polymer backbone and are (i) attached to the —O—R(ZD)-O— moiety as shown in formula (I) via a bioerodible moiety, and (ii) each formed from monomeric units that are coupled via bioerodible moieties;
   R is an optionally substituted hydrocarbon;
   Z is a linking group; and
   D is a releasable drug selected from a prostaglandin drug of general formulae (II) and (III):

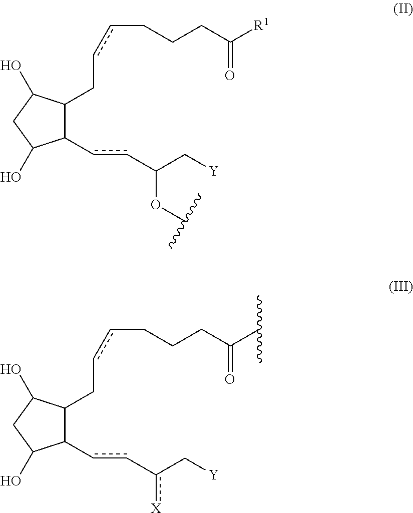

where [symbol] represents a double bond or single bond, [symbol] represents where the prostaglandin drug is attached to the linking group Z, R¹ is selected from —OH, —C₁₋₆alkoxy, and —C₁₋₆alkylamino, X is O or OH, and Y is selected from —(CH₂)₃CH₃, —OC₆H₄(meta-CF₃), (CH₂)₅CH₃, —OC₆H₅ and —CH₂C₆H₅.

Polymer-drug conjugates of the invention may optionally comprise a hydrophilic group. The hydrophilic group may be incorporated as a part of the polymer backbone structure. The hydrophilic group may be provided by or derived from, a monomer comprising at least one active-hydrogen group.

In some embodiments, the active-hydrogen group containing monomer may be selected from the groups consisting of poly(ethylene glycol), poly(lactic acid-co-glycolic acid) (PLGA) poly(1,5-dioxepan-2-one) (PDOO), poly(glycerol acetate), poly(hydroxy butyrate), poly(glycerol phosphate), amino acid polymers, amino acid oligomers, C₂ to C₄ diols, amino acids, glycolic acid, and hydroxy acids.

The polymer-drug conjugates in accordance with the invention can advantageously be prepared with a relatively high loading of drug, making them well suited to be formed into implants used at site within a subject that has a limited administration volume, for example the eye. This attribute, coupled with the activity of the drugs, makes the conjugates particularly suited for use as an ocular implant and in treating eye conditions, in particular glaucoma.

The present invention further provides a drug delivery system comprising a polymer-drug conjugate as described herein. The drug delivery system may comprise a hydrophilic component in combination with the polymer-drug conjugate. The hydrophilic component may be provided by (i) a hydrophilic group in the polymer backbone of the polymer-drug conjugate, (ii) a hydrophilic polymer in admixture with the polymer-drug conjugate, or (iii a combination thereof.

The present invention also provides an implant comprising a polymer-drug conjugate or a drug delivery system in accordance with the invention.

The present invention also provides an ocular implant comprising a polymer-drug conjugate or a drug delivery system in accordance with the invention.

The present invention further provides a method of treating an eye condition in a subject, said method comprising administering to the eye of the subject a polymer-drug conjugate or a drug delivery system in accordance with the invention. In that case, the polymer-drug conjugate or a drug delivery system will generally be provided in the form of an ocular implant.

The present invention also provides a process for preparing a polymer-drug conjugate comprising as part of its polymer backbone a moiety of general formula (I):

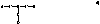

where:
A and B, which may be the same or different, represent the remainder of the polymer backbone and are (i) attached to the -J¹-R(ZD)-J²- moiety as shown in formula (I) via a bioerodible moiety, and (ii) each formed from monomeric units that are coupled via bioerodible moieties;
J¹ and J² are independently selected from the group consisting of oxygen, C(O) and NRa where R^{a} is hydrogen or C₁ to C₆ alkyl;
R is an optionally substituted hydrocarbon;
Z is a linking group;
D is a prostaglandin drug of formula (XX); and
D and Z together form an ester, anhydride or carbonate linking group, said process comprising a step of polymerising a drug-monomer conjugate of formula (V):

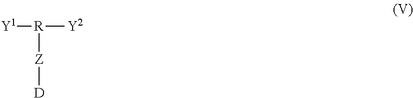

where:
Y¹ and Y² each independently represent a reactive functional group, or Y¹ and Y² together form part of a cyclic group capable of ring-opening; and R, Z and D are as defined above;
   with at least one monomer comprising compatible chemical functionality.

In some embodiments, Y¹ and Y¹ are each hydroxy.

A drug-monomer conjugate of general formula (V) has been found to be particularly versatile and can advantageously be polymerised with one or more other monomers using techniques well known in the art.

Monomers that are polymerised with the drug-monomer conjugate of formula (V) to form the polymer-drug conjugates of the invention will not only comprise compatible chemical functionality to react with the drug-monomer conjugate but that reaction will of course afford or give rise to a bioerodible moiety.

Through the polymerisation of a drug-monomer conjugate of formula (V), the process of the invention may advantageously be used to synthesise a polymer-drug conjugate with a high loading of one or more drugs.

Implants suitable for administration to the eye to deliver a therapeutic dose of drug may then be formed from the resulting polymer-drug conjugate or from materials that contain the polymer-drug conjugate using techniques well known in the art.

The polymer-drug conjugate in accordance with the invention may form part of or be formed into an article or device per se or can be presented as a coating on a preformed article or device.

The polymer-drug conjugates provide an effective and efficient means for delivering drugs to a subject.

In another aspect, the invention provides a method of delivering a drug to a subject, the method comprising administering to the subject a polymer-drug conjugate or a drug delivery system in accordance with the invention.

In another aspect, the invention provides a method for treating glaucoma in an animal subject suffering glaucoma in one or both eyes, the method comprising administering to an eye afflicted with glaucoma a polymer-drug conjugate or a drug delivery system in accordance with the invention.

In another aspect the invention provides use of a polymer-drug conjugate or use of a drug delivery system in accordance with the invention in manufacture of a medicament for the treatment of glaucoma in at least one eye of a subject.

Further aspects of the invention appear below in the detailed description of the invention.

### BRIEF DESCRIPTION OF THE FIGURES

Preferred embodiments of the invention will herein be illustrated by way of example only with reference to the accompanying drawings in which:

FIG. 1 is a graph showing the cumulative amount of latanoprost free acid (μg) released from polymer-drug conjugates in accordance with embodiments of the invention, over a period of up to 61 days.

### DETAILED DESCRIPTION OF THE INVENTION

The polymer-drug conjugates in accordance with the invention may be used in the treatment, cure, prevention, or diagnosis of disease in a subject, or used to otherwise enhance physical or mental well-being of a subject.

The polymer-drug conjugates in accordance with the invention can therefore be prepared such that they are suitable for administration to a subject (i.e. suitable for in vivo applications).

The invention provides a method of delivering a drug to a subject, the method comprising administering to the subject a polymer-drug conjugate in accordance with the invention.

By the conjugates being “suitable” for administration to a subject is meant that administration of the conjugate to a subject will not result in unacceptable toxicity, including allergenic responses and disease states.

By the term “subject” is meant either an animal or human subject. By “animal” is meant primates, livestock animals (including cows, horses, sheep, pigs and goats), companion animals (including dogs, cats, rabbits and guinea pigs), and captive wild animals (including those commonly found in a zoo environment). Laboratory animals such as rabbits, mice, rats, guinea pigs and hamsters are also contemplated as they may provide a convenient test system. Generally, the subject will be a human subject.

By “administration” of the polymer-drug conjugate to a subject is meant that the conjugate is transferred to the subject such that the drug will be released. Provided the drug can be released, there is no particular limitation on the mode of administration.

Where the polymer-drug conjugate is to be used to treat an eye condition in a subject, administration will generally be by way of intracameral, episcleral or subconjunctival administration. By “eye condition” is meant glaucoma, ocular hypertension or hypotrichosis.

The polymer-drug conjugates may be provided in particulate form and blended with a pharmacologically acceptable carrier to facilitate administration. By “pharmacologically acceptable” is meant that the carrier is suitable for administration to a subject in its own right.

In other words, administration of the carrier to a subject will not result in unacceptable toxicity, including allergenic responses and disease states. The term “carrier” refers to the vehicle with which the conjugate is contained prior to being administered.

As a guide only, a person skilled in the art may consider “pharmacologically acceptable” as an entity approved by a regulatory agency of a federal or state government or listed in the US Pharmacopeia or other generally recognised pharmacopeia for use in animals, and more particularly humans.

Suitable pharmacologically acceptable carriers are described in Martin, Remington's Pharmaceutical Sciences, 18th Ed., Mack Publishing Co., Easton, Pa., (1990), and include, but are not limited to, liquids that may be sterilised such as water and oils, including those of petroleum, animal, vegetable or synthetic origin, such as peanut oil, soya bean oil, mineral oil, sesame oil, and the like.

The conjugate may also form part of or be formed into an article or device, or be applied as a coating on an article or device, and implanted in a subject. By being “implanted” is meant that the article or device is totally or partly introduced medically into a subject's body, or by medical intervention into a natural orifice of a subject, and which is intended to remain there after the procedure. Where the article or device is to be implanted, it can conveniently be referred to as an “implant”.

Accordingly the invention provides an implant comprising a polymer-drug conjugate in accordance with the invention. Where the implant is to be administered to the eye, it may be conveniently referred to as an “ocular implant”. In that case, the ocular implant will generally be administered to a subject intracamerally, episclerally or subconjunctivally.

The polymer-drug conjugates or implants in accordance with the invention may be administered in a single dose or a series of doses.

The polymer-drug conjugate in accordance with the invention comprises a polymer backbone to which is conjugated a prostaglandin drug of general formulae (XX).

As used herein the term “conjugate” refers to the product formed through covalent bonding between the monomer or polymer and the drugs as depicted in formulae (I) and (V).

Accordingly, the term “conjugated” refers to the state of the product that is formed through covalent bonding between the monomer or polymer and the drugs as depicted in formulae (I) and (V).

In one aspect, the present invention relates to a polymer-drug conjugate comprising a polymer backbone and a prostaglandin or substituted prostaglandin conjugated to the polymer backbone via an ester, anhydride or carbonate linking group.

A “prostaglandin” is a drug typically derived from C20 prostanoic acid illustrated below:

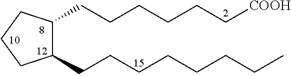 [SMILES: CCCCCCCC[C@H]1CCC[C@@H]1CCCCCCC(=O)O]

As used herein the term “prostaglandin” generally refers to an endogenously sourced prostaglandin drug. An example of a prostaglandin is PGF_{2α} (dinoprost).

As used herein the term “substituted prostaglandin” generally refers to a synthetic molecule derived from C₂₀ prostanoic acid, which is designed to bind to or interfere with a prostaglandin receptor. Substituted prostaglandins can be in the form of a therapeutically active drug or a prodrug. An example of a substituted prostaglandin is latanoprost. Substituted prostaglandins described herein may also be known as prostaglandin analogues.

Prostaglandins and substituted prostaglandins used in the present invention (also referred to herein as the “prostaglandin drug”) are conjugated pendant to the polymer backbone. That is, the conjugated drug does not form part of the polymer backbone chain. The pendant configuration ensures efficient release of the drug. Furthermore, by being pendant, the drug can be released without causing a reduction in the chain length of the polymer backbone.

The prostaglandins and substituted prostaglandins may be conjugated in free acid or prodrug form.

In general, the term “drug” refers to a substance for therapeutic use whose application (or one or more applications) involves: a chemical interaction, or physico-chemical interaction, with a subject's physiological system; or an action on an infectious agent, or on a toxin or other poison in a subject's body, or with biological material such as cells in vitro.

In general, a “prodrug” is a derivative of a bioactive agent, wherein the derivative may have little or none of the activity of the bioactive agent per se yet is capable of being converted into a bioactive agent or therapeutically active drug in vivo or in vitro.

As used herein, the term “prostaglandin drug” refers to a conjugated prostaglandin or substituted prostaglandin, or a pharmaceutically acceptable salt thereof, or a prodrug thereof, which is linked to the polymer backbone. The present invention enables the prostaglandin or a substituted prostaglandin, or pharmaceutically acceptable salt thereof, or prodrug thereof, to be delivered to a desired site in order to produce a therapeutic effect.

Accordingly, the term “prostaglandin drug” as used herein refers to free acid forms (including pharmaceutically acceptable salts thereof) and prodrug forms of the prostaglandins and substituted prostaglandins that are conjugated to the polymer backbone.

In one aspect, the present invention relates to a polymer-drug conjugate comprising a polymer backbone and a PGE, PGD and PGF class of substituted prostaglandin conjugated to the polymer backbone via an ester, anhydride or carbonate linking group. The PGF prostaglandin may be a substituted PGF_{α} or PGFβ prostaglandin. Preferably, the polymer-drug conjugate comprises a PGF_{α} class of substituted prostaglandin.

Prostaglandins and substituted prostaglandins as described herein constitute a α-chain, a ω-chain and a 5-membered ring, numbered according to the basic skeleton as follows:

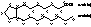 [SMILES: CCCCCCCC[C@H]1CCC[C@@H]1CCCCCCC(=O)O]

The prostaglandins and substituted prostaglandins are conjugated to the polymer backbone via an ester linking group, an anhydride linking group or a carbonate linking group at the 1, 9, 11 or 15 positions of the prostaglandin or substituted prostaglandin. The present invention has found that ester, anhydride and carbonate linking groups can help to ensure that a sufficient amount of the drug is effectively released from the polymer conjugate to achieve therapeutic levels in the immediate vicinity of the polymer conjugate material. As discussed further below, such linkages have also been found to provide for drug release with a zero order release profile. One advantage of the invention is that zero order release of the drug without a burst effect can be sustained over a period of time, such as over a period of at least 7 days, preferably over at least 30 days and more preferably over at least 90 days.

The present invention employs ester, anhydride and carbonate linking groups to conjugate the prostaglandin drug to the polymer backbone as such linking groups have been found to be hydrolytically labile in biological environments. As discussed further below, such linking groups are generally more labile than other groups or moieties that may be present in the polymer-drug conjugate, such as for example, bioerodible moieties that may be present in the polymer backbone of polymer-drug conjugates of some embodiments of the invention.

Prostaglandins and substituted prostaglandins delivered by polymer-drug conjugates of the invention comprise at least one functional group selected from the group consisting of a carboxylic acid group at the 1 position, a hydroxy group at the 9 position, a hydroxy group at the 11 position, and a hydroxy group at the 15 position.

The carboxylic acid group at the 1 position, and the hydroxy groups at the 9, 11 and 15 position of the prostaglandin or substituted prostaglandin can serve as reactive functional groups for conjugation of the prostaglandin drug to a polymer. In conjugating the drug to the polymer backbone, the prostaglandin drug is covalently linked to the polymer via the selected group at the 1, 9, 11 or 15 position. The drug moiety (denoted D in formulae described herein) linked to the polymer is therefore an acid residue (in the case of conjugation at the 1 position) or an alcohol residue (in the case of conjugation at the 9, 11 or 15 positions) of the ester, anhydride or carbonate linking group conjugating the prostaglandin drug to the polymer backbone. The drug moiety represented by D may be a releasable prostaglandin or a releasable substituted prostaglandin.

When the prostaglandin or substituted prostaglandin is conjugated to the polymer backbone by an ester linking group, the ester linking group may link the drug at a position selected from the group consisting of the 1, 9, 11 and 15 position of the prostaglandin or substituted prostaglandin.

When the prostaglandin or substituted prostaglandin is conjugated to the polymer backbone by an anhydride linking group, the anhydride linking group may link the drug at the 1 position of the prostaglandin or substituted prostaglandin.

When the prostaglandin or substituted prostaglandin is conjugated to the polymer backbone by a carbonate linking group, the carbonate linking group may link the drug at a position selected from the group consisting of the 9, 11 and 15 position of the prostaglandin or substituted prostaglandin.

The “acid residue” is a reference to that part of the ester or anhydride linking group derived from the carboxylic acid functional group of the drug after conjugation of the prostaglandin drug to the polymer backbone. The carboxylic acid group is located at the 1 position. The acid residue will generally have the structure —C(O)O—

The “alcohol residue” is a reference to that part of the ester or carbonate linking group derived from a hydroxy functional group of the drug after conjugation of the prostaglandin drug to the polymer backbone. The hydroxy group may be selected by located at the 9, 11 or 15 position. The alcohol residue will generally have the structure —O—.

Polymer-drug conjugates of the invention comprise at least one prostaglandin drug conjugated to the polymer backbone. More typically, polymer-drug conjugate of the invention comprise a plurality of prostaglandin drugs.

In some embodiments, the polymer-drug conjugate comprises a plurality of prostaglandin drugs of formula (XX):

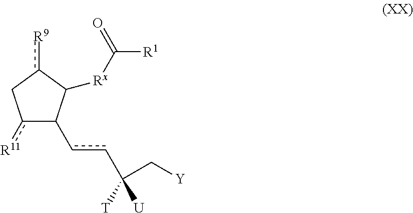

where:
R^{x} is a straight chain aliphatic of six carbon atoms optionally comprising one or two substituents selected from the group consisting of oxo (═O) and hydroxy;
[symbol] represents a double or single bond;
T and U are selected from the group consisting of where T and U together form oxo (═O), where T and U are each halo, and where T is R¹⁵ and U is hydrogen;
Y is optionally substituted C4 to C10 hydrocarbyl or optionally substituted C₄ to C₁₀ hydrocarbyloxy; and
one of R¹, R⁹, R¹¹ and R¹⁵ is linked to the polymer backbone and wherein:
R⁹, R¹¹ and R¹⁵ when linked to the polymer backbone are the alcohol residue of an ester or carbonate linking group and R¹ when linked to the polymer backbone forms the acid residue of an ester or anhydride linking group; and
R¹ when not linked to the backbone is selected from the group consisting of —OH, —O(C₁₋₆ alkyl), and —NR^{a}R^{b} where R^{a} and R^{b} are each independently selected from the group consisting of H and C₁₋₆ alkyl;
R⁹ and R¹¹ when not linked to the polymer backbone are both hydroxy or one is hydroxy and one is oxo and where one of R⁹ and R¹¹ is linked to the backbone, the other is hydroxy or oxo; and
when R¹⁵ is not linked to the backbone then T is hydroxy and U is hydrogen, or T and U are each fluoro, or T and U together form oxo.

The plurality of prostaglandin drugs present in polymer-drug conjugates of the invention may each be of the same type, or they may be a mixture of two or more different types of prostaglandin drug.

In some embodiments of formula (XX), R^{x} comprises zero or one substituent selected from oxo or hydroxy, wherein the oxo or hydroxy is present in the 6 position of the prostaglandin drug. That is, Rx may be unsubstituted, or it may contain one oxo or one hydroxy substituent, which is located at the 6 position of the prostaglandin drug.

In some embodiments, polymer-drug conjugate of the invention comprise a plurality of prostaglandin drugs of formula (XXi):

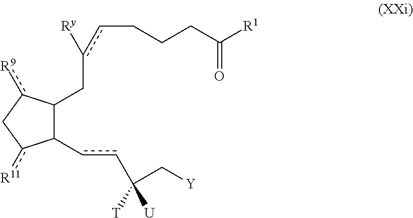

where:
[symbol] represents a double or single bond;
T and U are selected from the group consisting of where T and U together form oxo (═O), where T and U are each halo, and where T is R¹⁵ and U is hydrogen;
R^{y} is an optional substituent selected from the group consisting of oxo and hydroxy;
Y is optionally substituted C4 to C10 hydrocarbyl or optionally substituted C₄ to C₁₀ hydrocarbyloxy; and
one of R¹, R⁹, R¹¹ and R¹⁵ is linked to the polymer backbone and wherein:
R⁹, R¹¹ and R¹⁵ when linked to the polymer backbone are the alcohol residue of an ester or carbonate linking group and R¹ when linked to the polymer backbone forms the acid residue of an ester or anhydride linking group; and
R¹ when not linked to the backbone is selected from the group consisting of —OH, —O(C₁₋₆ alkyl), and —NR^{a}R^{b} where R^{a} and R^{b} are each independently selected from the group consisting of H and C₁₋₄ alkyl;
R⁹ and R¹¹ when not linked to the polymer backbone are both hydroxy or one is hydroxy and one is oxo and where one of R⁹ and R¹¹ is linked to the backbone, the other is hydroxy or oxo; and
when R¹⁵ is not linked to the backbone then T is hydroxy and U is hydrogen, or T and U are each fluoro, or T and U together form oxo.

In prostaglandin drugs of formulae (XX) or (XXi), Y is optionally substituted C₄ to C₁₀ hydrocarbyl or optionally substituted C₄ to C₁₀ hydrocarbyloxy. The hydrocarbyl (including the hydrocarbyl portion of the hydrocarbyloxy) may comprise aliphatic, alicyclic or aromatic hydrocarbon groups or combinations thereof.

In some embodiments of formulae (XX) and (XXi), Y is optionally substituted with one or more substituents selected from halo and halo-C₁ to C₄ alkyl. Suitable halo may be fluoro, chloro, bromo or iodo. Preferred halo is fluoro. Halo-C₁ to C₄ alkyl may be perhalomethyl, such as for example, trifluoromethyl.

In some embodiments, Y is selected from the group consisting of C₄ to C₁₀ alkyl, C₄ to C₁₀ alkoxy, phenyl, phenyl substituted C₁ to C₄ alkyl, and phenyl substituted C₁ to C₄ alkoxy, wherein the groups are optionally substituted with one or more groups selected from halo and perhalomethyl. In some specific embodiments, Y is selected from the group consisting of —(CH₂)₃CH₃, —OC₆H₄(meta-CF₃), —(CH₂)₅CH₃, —O(C₆H₅) and —CH₂(C₆H₅).

In formulae (XX) and (XXi), T and U represent substituent groups present on the substituted prostaglandin. In some embodiments, T and U together form an oxo (═O) substituent group.

In other embodiments, T and U are each halo substituent groups. Suitable halo may be fluoro, chloro, bromo or iodo. Preferred halo is fluoro. In other embodiments, T is R¹⁵ and U is hydrogen.

In accordance with the invention, the prostaglandin drug is linked to the polymer backbone by one of R¹, R⁹, R¹¹ and R¹⁵. Accordingly, when linked to the polymer backbone, R⁹, R¹¹ and R¹⁵ represent the alcohol residue (—O—) of an ester or carbonate linking group, and R¹ forms the acid residue (—C(O)O—) of an ester or anhydride linking group.

In some embodiments, R¹ is linked to the polymer backbone via an ester linkage or an anhydride linkage. In such embodiments, R⁹, R¹¹ and R¹⁵ are not linked to the polymer backbone.

In some embodiments, R⁹ is linked to the polymer backbone via an ester linkage or a carbonate linkage. In such embodiments, R¹, R¹¹ and R¹⁵ are not linked to the polymer backbone.

In some embodiments, R¹¹ is linked to the polymer backbone via an ester linkage or a carbonate linkage. In such embodiments, R¹, R⁹ and R¹⁵ are not linked to the polymer backbone.

In some embodiments, R¹⁵ is linked to the polymer backbone via an ester linkage or a carbonate linkage. In such embodiments, R¹, R⁹ and R¹¹ are not linked to the polymer backbone.

One skilled in the art would understand that when R¹, R⁹, R¹¹ and R¹⁵ are not linked to the polymer backbone, then these groups may represent substituent groups.

R¹ when not linked to the polymer backbone may together with the carbonyl group (—C(O)—), be a carboxylic acid group, or an ester or amide derivative thereof. In some embodiments, R¹ when not linked to the polymer backbone is selected from the group consisting of —OH, —O(C₁₋₆alkyl), and —NR^{a}R^{b} where R^{a} and R^{b} are each independently selected from the group consisting of H and C₁₋₆alkyl. In specific embodiments, R¹ when not linked to the polymer backbone is selected from the group consisting of —OH, —O(iso-propyl) and —NHethyl.

R⁹ and R¹¹ when not linked to the polymer backbone are selected from the group consisting of hydroxy and oxo. In some embodiments, when one of R⁹ and R¹¹ is linked to the backbone, the other of R⁹ and R¹¹ is hydroxy or oxo. In other embodiments, when both R⁹ and R¹¹ are not linked to the polymer backbone, then R⁹ and R¹¹ are both hydroxy. In other embodiments, one of R⁹ and R¹¹ is hydroxy and the other of R⁹ and R¹¹ is oxo.

When R¹⁵ is not linked to the polymer backbone then T and U may each represent hydrogen or a substituent group, or T and U together may form a substituent group. In some embodiments, T is hydroxy and U is hydrogen. In other embodiments, T and U are each halo (preferably fluoro). In yet other embodiments, T and U together form oxo.

In some embodiments, the polymer-drug conjugate of the invention comprises a prostaglandin drug of formula (XXii):

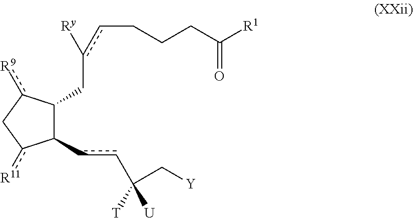

wherein R^{y}, R¹, R⁹, R¹¹, Y, T and U are as defined.

In some embodiments, the prostaglandin drug (D) is selected from the group consisting of:

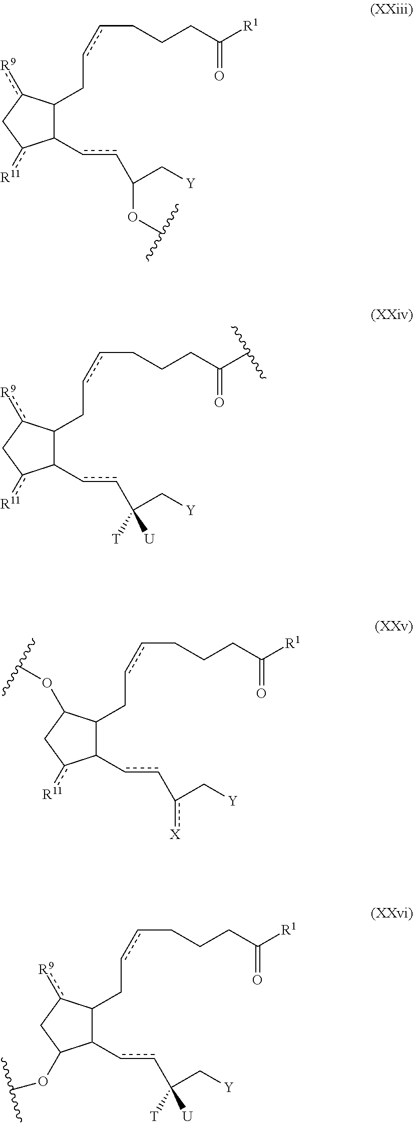

wherein:
[symbol] represents the point of attachment of the prostaglandin drug to linking group Z;
[symbol] represents a double or single bond;
Y is optionally substituted C4 to C10 hydrocarbyl or optionally substituted C4 to C10 hydrocarbyloxy;
in formulae (XXiii), (XXv) and (XXvi) R¹ is hydroxy, C₁ to C₆ alkoxy or C₁ to C₆ alkylamino (preferably, isopropoxy or ethylamino);
in formulae (XXiii) and (XXiv) R⁹ and R¹¹ are hydroxy or one of R⁹ and R¹¹ is oxo and the other is hydroxy;
in formula (XXv) R¹¹ is hydroxy or oxo and X is O or hydroxy;
in formula (XXvi) R⁹ is hydroxy or oxo; and
in formulae (XXiv) and (XXvi) T is hydroxy and U is hydrogen, or T and U are both fluoro, or T and U together form oxo.

A skilled person would be able to ascertain the chemical structure of a variety of prostaglandins and substituted prostaglandins. Prostaglandin drugs conjugated to polymer-drug conjugates of the invention may be in free acid form (including pharmaceutically acceptable salts thereof) or prodrug form.

By “free acid” form is meant that prostaglandins and substituted prostaglandins as described herein may present as a “free” carboxylic acid (i.e. COOH) or be conjugated to the polymer backbone through that free carboxylic acid group at the 1 position of the prostaglandin drug.

The free carboxylic acid group is generally in the α-chain of the prostaglandin or substituted prostaglandin. In such cases, the prostaglandin drug is releasable, or can be released, in its free acid form. The free acid form may optionally be associated with a pharmaceutically acceptable salt.

Prostaglandins and substituted prostaglandins in free acid form may also be conjugated through a hydroxy group at the 9, 11 or 15 position of the prostaglandin or substituted prostaglandin. In such embodiments, the prostaglandin or substituted prostaglandin is also releasable, or can be released, in its free acid form. The free acid form may optionally be associated with a pharmaceutically acceptable salt.

When the prostaglandin drug is present as the prodrug, the prostaglandin drug will generally be conjugated through a hydroxy group at the 9, 11 or 15 position. In such cases, the prostaglandin drug is releasable, or can be released, in its prodrug form.

The term “pharmaceutically acceptable salt” means those salts that are safe and effective for use in pharmaceutical preparations. Pharmaceutically acceptable salts include salts of acidic groups present in compounds of the invention. Suitable salts may include sodium, potassium, ammonium, calcium, diethylamine and piperazine salts and the like. Pharmaceutically acceptable salts are described in Stahl P H, Wermuth C G, editors. 2002. Handbook of pharmaceutical salts: Properties, selection and use. Weinheim/Zurich: Wiley-VCH/VHCA.

Prostaglandins and substituted prostaglandins as described herein may present as a prodrug, wherein the carboxylic acid at the 1 position is substituted with a labile substituent group that is removable in vivo. In such cases, the prostaglandin or substituted prostaglandin will be conjugated to the polymer backbone through a hydroxy group at the 9, 11 or 15 position. In such cases, the prostaglandin drug is releasable, or can be released, in its prodrug form. A prodrug may be an ester or amide derivative of the free acid form of the drug. The prodrug can be converted into the free acid form in vivo. For example, latanoprost, travoprost, tafluprost and bimatoprost are prodrugs, and are converted to their free acid forms in vivo.

Some examples of prostglandins and substituted prostaglandins that may be delivered by the polymer-drug conjugates are shown in Table 1. For further clarification as to what is meant by the “free acid form” of prostaglandins, the following illustrates the differences in chemical structure between some prodrugs and their respective free acid forms. Such drugs (either in prodrug or free acid form) are conjugated to the polymer backbone of the polymer-drug conjugates of the invention by one of the functional groups located at the 1, 9, 11 or 15 positions of the prostaglandin or substituted prostaglandin, and may be delivered in free acid or prodrug form.

TABLE 1

| Pro-drug form | Free-acid form |
|---|---|
| | 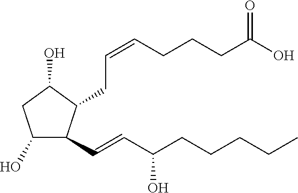 [SMILES: CCCCC[C@H](O)/C=C/[C@@H]1[C@@H](C/C=C\CCCC(=O)O)[C@@H](O)C[C@H]1O] |
| | PGF2α (dinoprost) |
| | 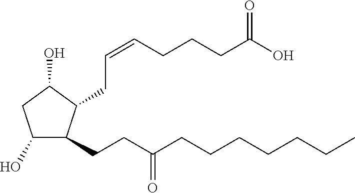 [SMILES: CCCCCCCC(=O)CC[C@@H]1[C@@H](C/C=C\CCCC(=O)O)[C@@H](O)C[C@H]1O] |
| | Unoprostone |
| 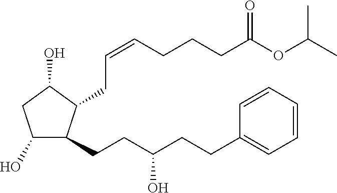 [SMILES: CC(C)OC(=O)CCC/C=C\C[C@@H]1[C@@H](CC[C@@H](O)CCc2ccccc2)[C@H](O)C[C@@H]1O] | 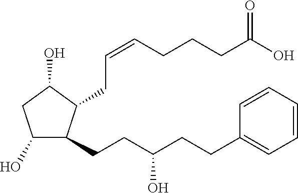 [SMILES: O=C(O)CCC/C=C\C[C@@H]1[C@@H](CC[C@@H](O)CCc2ccccc2)[C@H](O)C[C@@H]1O] |
| Latanoprost | Free acid form of Lantanoprost |
| 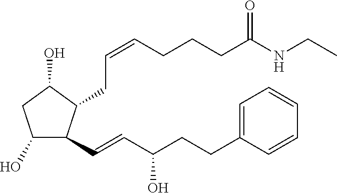 [SMILES: CCNC(=O)CCC/C=C\C[C@@H]1[C@@H](/C=C/[C@@H](O)CCc2ccccc2)[C@H](O)C[C@@H]1O] | 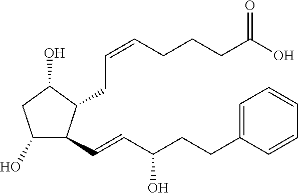 [SMILES: O=C(O)CCC/C=C\C[C@@H]1[C@@H](/C=C/[C@@H](O)CCc2ccccc2)[C@H](O)C[C@@H]1O] |
| Bimatoprost | Free acid form of Bimatoprost |
| 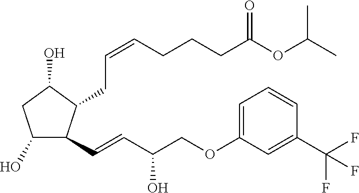 [SMILES: CC(C)OC(=O)CCC/C=C\C[C@@H]1[C@@H](/C=C/[C@@H](O)COc2cccc(C(F)(F)F)c2)[C@H](O)C[C@@H]1O] | 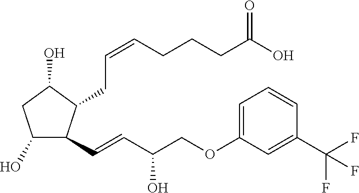 [SMILES: O=C(O)CCC/C=C\C[C@@H]1[C@@H](/C=C/[C@@H](O)COc2cccc(C(F)(F)F)c2)[C@H](O)C[C@@H]1O] |
| Travoprost | Fluprostenol |
| 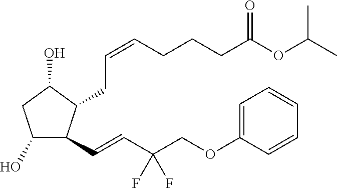 [SMILES: CC(C)OC(=O)CCC/C=C\C[C@@H]1[C@@H](/C=C/C(F)(F)COc2ccccc2)[C@H](O)C[C@@H]1O] | 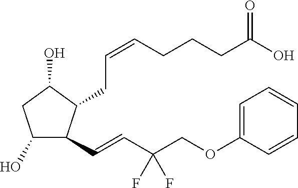 [SMILES: O=C(O)CCC/C=C\C[C@@H]1[C@@H](/C=C/C(F)(F)COc2ccccc2)[C@H](O)C[C@@H]1O] |
| Tafluprost | Free acid form of tafluprost |

Drugs such as latanoprost, travoprost, bimatoprost and tafluprost are substituted prostaglandins. However such drugs are not formulated in eye drops in their “free acid” form but rather are formulated as prodrugs, being ester or amide derivatives of the free acid form. This is because the free acid form is not bioavailable when delivered in an eye drop formulation.

Accordingly, it will be convenient in the context of the present invention to refer to the prostaglandin drugs of general formulae (XX) or (XXi) as the free acid form of other prostaglandins. For example the free acid form of latanoprost is ((Z)-7-[(1R,2R,3R,5S)-3,5-dihydroxy-2-[(3R)-3-hydroxy-5-phenylpentyl]-cyclopentyl]hept-5-enoic acid.

Prostaglandin drugs such as dinoprost (PGF2_{α}) are naturally occurring compounds, and exist in their free acid form.

Specific examples of releasable prostaglandin drugs of formulae described herein include latanoprost, travoprost, bimatoprost and tafluprost, the free acid form of latanoprost, travoprost (known as fluprostenol), bimatoprost and tafluprost, as well as unoprostone and dinoprost.

In some embodiments it is preferable that the prostaglandin drug be releasable, or be released, in free acid form. In some embodiments of this invention, it is preferred that the releasable prostaglandin drug be selected from the free acid form of latanoprost and the free acid form of travoprost. The free acid form of latanoprost is most preferred.

Although not necessarily depicted, those skilled in the art will appreciate that the prostaglandins and substituted prostaglandins of general formulae described herein will have particular stereoisomeric structures, and possibly particular geometric isomeric structures.

For avoidance of any doubt, the prostaglandins and substituted prostaglandins of general formulae described herein are intended to embrace all such structures.

In another aspect, the present invention relates to a polymer-drug conjugate of formula (X) comprising a polymer backbone and a plurality of prostaglandin drugs conjugated to the polymer backbone via an ester, anhydride or carbonate linking group:

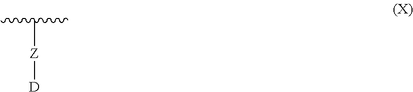

where:
[symbol] represents a polymer backbone;
Z is a linking group;
D is a prostaglandin drug of formula (XX); and
D and Z together form an ester, anhydride or carbonate linking group.

In some embodiments, when prostaglandin drugs of formula (XX) are conjugated to the polymer backbone at R¹ via an ester linking group or an anhydride linking group, the polymer-drug conjugate of formula (X) has a structure of formula (Xa):

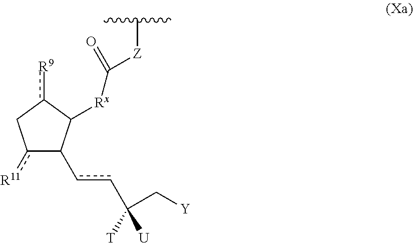

wherein:
[symbol] represents a polymer backbone;
Z is a linking group; and
Z and the prostaglandin drug of formula (XX) together form an ester or anhydride linking group.

In some embodiments, when prostaglandin drugs of formula (XX) are conjugated to the polymer backbone at R⁹ via an ester linking group or a carbonate linking group, the polymer-drug conjugate of formula (X) has a structure of formula (Xb):

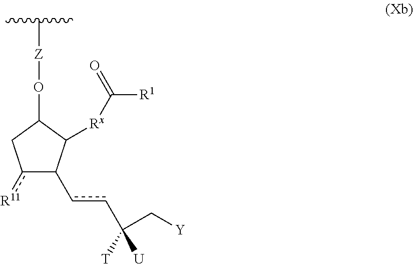

wherein:
[symbol] represents a polymer backbone;
Z is a linking group; and
Z and the prostaglandin drug of formula (XX) together form an ester or carbonate linking group.

In some embodiments, when prostaglandin drugs of formula (XX) are conjugated to the polymer backbone at R¹¹ via an ester linking group or a carbonate linking group, the polymer-drug conjugate of formula (X) has a structure of formula (Xc):

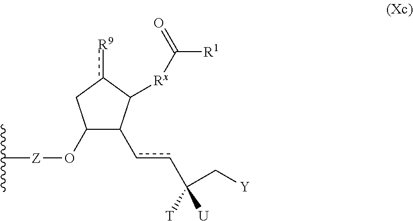

wherein:
[symbol] represents a polymer backbone;
Z is a linking group; and
Z and the prostaglandin drug of formula (XX) together form an ester or carbonate linking group.

In some embodiments, when prostaglandin drugs of formula (XX) are conjugated to the polymer backbone at R¹⁵ via an ester linking group or a carbonate linking group, the polymer-drug conjugate of formula (X) has a structure of formula (Xd):

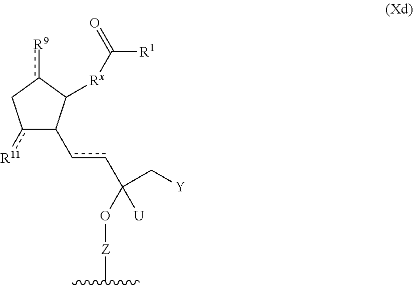

wherein:
Z is a linking group; and
Z and the prostaglandin drug of formula (XX) together form an ester or carbonate linking group.

In formula (Xa), the prostaglandin drug of formula (XX) is coupled to the polymer backbone by the group —Z—. The prostaglandin drug of formula (XX) and Z together form an ester, anhydride or carbonate linking group. In formula (Xa), the prostaglandin drug is therefore covalently linked to the oxygen atom that is part of Z to form part of an ester linkage (ester bond) or an anhydride linkage (anhydride bond).

When the molecule of formula (XX) and Z form an ester or anhydride linking group, the prostaglandin drug will comprise the acid residue of the ester or anhydride linking group, while Z will comprise the alcohol residue of the ester or anhydride linking group. Upon hydrolysis or cleavage of the ester or anhydride linking group, a carboxylic acid group will then form on the prostaglandin or substituted prostaglandin, while an alcohol (—OH) group will form on Z.

In formulae (Xb), (Xc) and (Xd), the prostaglandin drug of formula (XX) is coupled to the polymer backbone by the group —Z—. The prostaglandin drug of formula (XX) and Z together form an ester or carbonate linking group. In formulae (Xb), (Xc) and (Xd), the prostaglandin drug is covalently linked to the carbon atom of the —C(O)— moiety that is part of Z to form part of an ester linkage (ester bond) or an carbonate linkage (carbonate bond).

When the molecule of formula (XX) and Z form an ester or carbonate linking group, the prostaglandin drug will comprise the alcohol residue of the ester or carbonate linking group, while Z will comprise the acid residue of the ester or carbonate linking group. Upon hydrolysis or cleavage of the ester or carbonate linking group, an alcohol (—OH) group will then form on the prostaglandin or substituted prostaglandin, while a carboxylic acid group will form on Z.

In formulae (Xa, (Xb), (Xc) and (Xd), Z represents a linking group. Some specific embodiments of Z are described below.

In some embodiments, the polymer-drug conjugates in accordance with the invention are “bioerodible”. By being “bioerodible” is meant that the conjugates have a molecular structure that is susceptible to break down (i.e. a reduction in molecular weight) by chemical or enzymatic decomposition in a biological environment (e.g. within a subject or in contact with biological material such as blood, tissue etc), as opposed to physical degradation. Such decomposition will typically be via the hydrolysis of labile moieties that form part of the molecular structure of the conjugates. In other words, the conjugates will comprise moieties that are susceptible to hydrolytic cleavage. The rate of hydrolysis of the bioerodible polymer may vary over time, or be activated by any number of extrinsic or intrinsic factors (e.g. light, heat, radiation, pH, enzymatic or non-enzymatic cleavage, etc.).

Reference herein to biological material such as “biological tissue” is intended to include cells or tissue in vivo (e.g. cells or tissue of a subject) and in vitro (e.g. cultured cells).

In another aspect, the present invention relates to a bioerodible polymer-drug conjugate comprising as part of its polymer backbone a moiety of general formula (I):

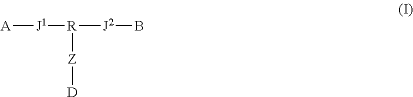

where:
A and B, which may be the same or different, represent the remainder of the polymer backbone and are (i) attached to the -J¹-R(ZD)-J²- moiety as shown in formula (I) via a bioerodible moiety, and (ii) each formed from monomeric units that are coupled via bioerodible moieties;
J¹ and J² are independently selected from the group consisting of oxygen, C(O), and NR^{a} where R^{a} is hydrogen or C₁ to C₆ alkyl;
R is an optionally substituted hydrocarbon;
Z is a linking group;
D is a prostaglandin drug of formula (XX); and
D and Z together form an ester, anhydride or carbonate linking group.

For avoidance of any doubt, the “moiety of general formula (I)” is intended to be a reference to:

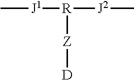

with [symbol] representing the connectivity to A and B, and A and B being presented in formula (I) to (i) more clearly depict that the “moiety” forms part of the polymer backbone, and (ii) define the nature of the remainder of the polymer backbone.

As used herein the expression forming “part of the polymer backbone” means that the moiety of formula (I) (i.e. excluding A and B) is part of the string of atoms that are each connected so as to form the polymer chain (i.e. including A and B). In other words, the moiety per se of formula (I) is not pendant from the polymer backbone. Having said this, it will be appreciated that groups Z and D in the moiety of formula (I) will be pendant from the polymer backbone.

Examples of A and B are discussed in more detail below, but include polyurethane and polyester polymer chains, as well as copolymers thereof.

Depending on the application, the polymer-drug conjugate may have a single moiety of formula (I), but more typically the conjugate will comprise a plurality of moieties of formula (I).

In polymers comprising a plurality of moieties of formula (I), each group represented by A, B, R, Z and D may be the same or different.

For example, the moiety of general formula (I) may in conjunction with a suitable comonomer form a repeat unit of a polyester or polyurethane as illustrated below in general formula (Ia) and (Ib), respectively:

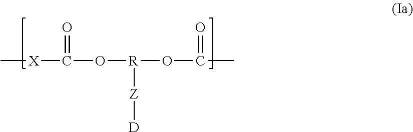

where J¹ and J² are each O, and R, Z, and D are as herein defined and X is an optionally substituted alkyl, aryl or alkylaryl group, wherein for each repeat unit of the polyester each R, Z, D and X may be the same or different;

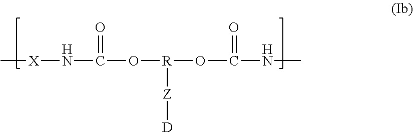

where J¹ and J² are each O, and R, Z and D are as herein defined and X is an optionally substituted alkyl, aryl or alkylaryl group, wherein for each repeat unit of the polyurethane each R, Z, D and X may be the same or different.

By being bioerodible, polymer-drug conjugates in accordance with one aspect of the invention can advantageously be used to release a prostaglandin drug moiety “D”, for example within a subject, without the need to subsequently remove the remaining conjugate structure from the subject.

Bioerodible polymer-drug conjugate will typically have multiple bioerodible moieties in its polymer backbone through which bioerosion can occur. Those skilled in the art will appreciate that the rate at which a particular bioerodible moiety in the polymer backbone undergoes hydrolytic cleavage under given environment relative to another can vary depending on the nature of each moiety (e.g. type of functionality, steric and electronic effects etc).

The same rationale can also apply to the rate at which the polymer backbone erodes relative to the rate of release of the drug.

An important feature of the bioerodible properties of the conjugates of one aspect of the invention is that (i) the -J¹-R(ZD)-J²- moiety as shown in formula (I) is attached to the remainder of the polymer backbone (represented by A and B) via a bioerodible moiety, and (ii) A and B are each formed from monomeric units that are coupled via a bioerodible moiety. By having such characteristics, the conjugates in accordance with the invention can advantageously fully bioerode.

As used herein the expression “bioerodible moiety” is intended to mean a moiety that can undergo chemical or enzymatic decomposition in a biological environment. Such chemical decomposition will typically be via hydrolysis. In other words, the bioerodible moiety with be susceptible to hydrolytic cleavage. In the context of the present invention, the bioerodible moieties function to link or couple the monomeric units that form the polymer backbone of the conjugates. Accordingly, it will be appreciated that the bioerodible moieties give rise to the bioerodible property of the conjugates.

Those skilled in the art will appreciate the type of moieties that are typically susceptible to hydrolytic cleavage in a biological environment. Such moieties may include anhydride, amide, urethane (carbamate), and ester. Bioerodible polymer-drug conjugates in accordance with the invention may include a combination of such moieties.

In accordance with some embodiments of the invention, A and B, which may be the same or different, represent the remainder of the polymer backbone and are “attached to the -J¹-R(ZD)-J²- moiety as shown in formula (I) via a bioerodible moiety”. By this is meant that the atoms represented by J¹ and J² in the -J¹-R(ZD)-J²- moiety each form part of a bioerodible moiety. For example, J¹ and J² in the -J¹-R(ZD)-J²- moiety may each represent O atoms and may each independently form part of an ester or urethane moiety as illustrated below where O* represents the O atom represented by J¹ and J²:

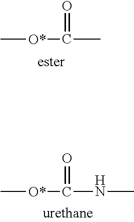

In one embodiment, the J¹ and J² atoms in the -J¹-R(ZD)-J²- each independently form part of an ester or urethane moiety.

A skilled person would understand that J¹ and J² can also form part of an ester or urethane moiety when J¹ and J² represent —C(O)— or NR¹ (where Ra is hydrogen or C1 to C6 alkyl), respectively.

In some embodiments of the invention of a bioerodible polymer-drug conjugate of the invention, it is preferred that the prostaglandin drug moiety (D) be released from the polymer-drug conjugate at a rate that is at least equal to or faster than the rate of cleavage of the bioerodible moieties forming part of the polymer backbone. That is, the linking group (Z) linking D to the polymer backbone should as labile, or more labile, than the bioerodible moieties forming part of the polymer backbone. Accordingly, drug release from the polymer-drug conjugate as a result of cleavage or hydrolysis of the ester, anhydride or carbonate linkage occurs at a rate that is at least equal to, or faster than, the rate of erosion of bioerodible moieties in the polymer backbone. In specific embodiments, it is preferred that the prostaglandin drug moiety (D) be released at a rate that is faster than the rate of erosion or degradation of the bioerodible moieties forming part of the polymer backbone.

When J¹ and J² form part of an ester moiety or urethane moiety, it is preferred that the ester or urethane moiety be less labile than the ester, anhydride or carbonate linkage conjugating the drug moiety (D) to the polymer backbone. In this manner, the conjugated drug can be released from the polymer conjugate free from fragments derived from the polymer backbone. In some embodiments, J¹ and J² form part of a urethane moiety.

Prostaglandins and substituted prostaglandins are releasable from polymer-drug conjugates of the invention. In polymer-drug conjugates of formulae described herein, by the prostaglandin drugs being “releasable” is meant that they are capable of being released or cleaved from the Z group defined in general formulae herein. Upon being released, the prostaglandin drug is bioactive or will be converted in vivo or in vitro to a bioactive form (e.g. as in the case of a prodrug).

In some embodiments, the polymer-drug conjugate comprises a plurality of moieties of formula (I), wherein each moiety of formula (I) comprises a prostaglandin drug (D) of formula (XX) linked to the polymer backbone via an ester, anhydride or carbonate linking group at one of R¹, R⁹, R¹¹ and R⁵ is of the prostaglandin drug.

In embodiments of the invention the prostaglandin drugs are released such that they do not comprise a residue derived from the polymer backbone or linker group (Z). By this it is meant that the drugs are released in their substantially original form (i.e. before being conjugated) and are essentially free from, for example, fragments of oligomer or polymer derived from the polymer backbone.

The prostaglandin drug may be released from the polymer-drug conjugate such that it provides for a sustained drug delivery system. Such a delivery system may in its simplest form be the conjugate provided in a desired shape, for example a rod or more intricate shape.

To promote surface area contact of the conjugate with a biological environment, the conjugate may also be provided in the form of a coating on substrate, or as an article have porosity (e.g. an open cell foam).

In one form of a polymer-drug conjugate comprising a moiety of formula (I), the prostaglandin drug (D) is of formula (XXii):

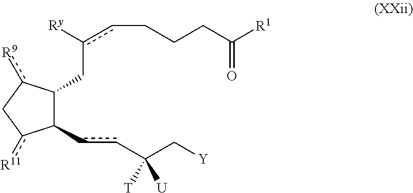

wherein R^{y}, R¹, R⁹, R¹¹, Y, T and U are as defined.

In some embodiments, D is a prostaglandin drug selected from the group consisting of:

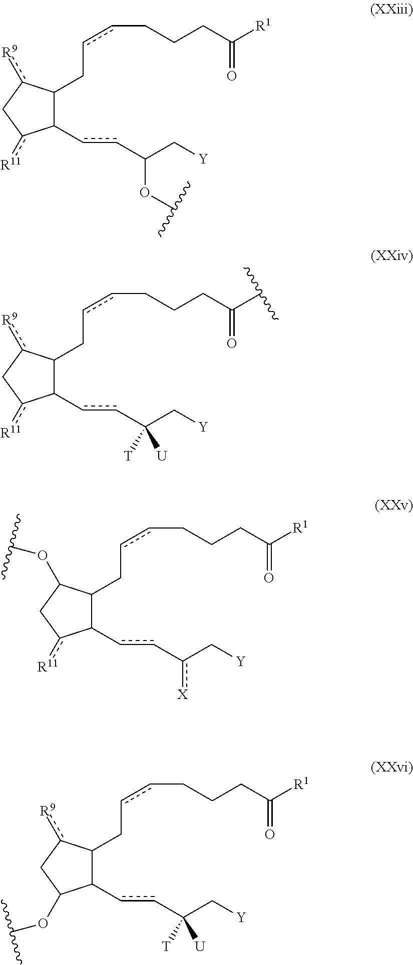

wherein:
[symbol] represents the point of attachment of the prostaglandin drug to linking group Z;
[symbol] represents a double or single bond;
Y is optionally substituted C4 to C10 hydrocarbyl or optionally substituted C₄ to C₁₀ hydrocarbyloxy;
in formulae (XXiii), (XXv) and (XXvi) R¹ is hydroxy, C₁ to C₆ alkoxy (preferably isopropoxy) or C₁ to C₈alkylamino (preferably ethylamino);
in formulae (XXiii) and (XXiv) R⁹ and R¹¹ are hydroxy or one of R⁹ and R¹¹ is oxo and the other is hydroxy;
in formula (XXv) R¹¹ is hydroxy or oxo and X is O or hydroxy; in formula (XXvi) R⁹ is hydroxy or oxo; and in formulae (XXiv) and (XXvi) T is hydroxy and U is hydrogen, or T and U are both fluoro, or T and U together form oxo.

In some embodiments, D is a prostaglandin drug of the following formula:

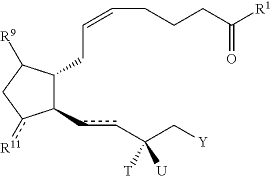

where R¹, R⁹, R¹¹, T, U and Y are as herein described.

In another aspect, the present invention relates to a bioerodible polymer-drug conjugate comprising as part of its polymer backbone a moiety of general formula (Ic):

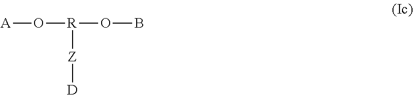

where:
A and B, which may be the same or different, represent the remainder of the polymer backbone and are (i) attached to the —O—R(ZD)-O— moiety as shown in formula (Ic) via a bioerodible moiety, and (ii) each formed from monomeric units that are coupled via bioerodible moieties;
R is an optionally substituted hydrocarbon;
Z is a linking group;
D is a prostaglandin drug of formula (XX); and
D and Z together form an ester, anhydride or carbonate linking group.

The present invention further relates to a bioerodible polymer-drug conjugate comprising as part of its polymer backbone a moiety of general formula (Ic):

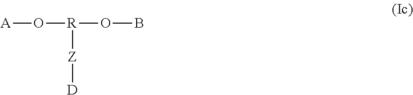

where:
A and B, which may be the same or different, represent the remainder of the polymer backbone and are (i) attached to the —O—R(ZD)-O— moiety as shown in formula (Ic) via a bioerodible moiety, and (ii) each formed from monomeric units that are coupled via bioerodible moieties;
R is an optionally substituted hydrocarbon;
Z is a linking group; and
D is a releasable drug selected from prostaglandin drugs of general formulae (II) and (III):

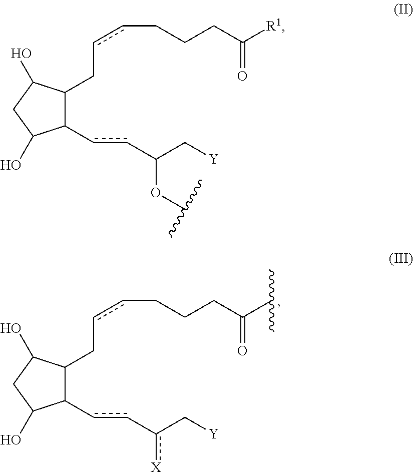

where:
[symbol] represents a double or single bond, [symbol] represents where the prostaglandin drug is attached to the linking group Z, R¹ is selected from OH, C₁₋₆ alkoxy (preferably iso-propyloxy) and C₁-C₆ alkylamino (preferably ethylamino), X is O or OH, and Y is selected from —(CH₂)₃CH₃, —OC₆H₄(meta-CF₃), (CH₂)₅CH₃, —O(C₆H₅), and —CH₂(C₆H₅).

In some embodiments of formula (II), R¹ is hydroxy.

In order for the prostaglandin drug (denoted by D) to be released, the covalent bond between D and the Z group will of course need to be cleaved.

Cleavage of the covalent bond between the D and Z group can be promoted hydrolytically (i.e. hydrolytic cleavage) and may take place in the presence of water and an acid or a base. In some embodiments the cleavage may take place in the presence of one or more hydrolytic enzymes or other endogenous biological compounds that catalyze or at least assist in the cleavage process. For example, an ester bond may be hydrolytically cleaved to produce a carboxylic acid and an alcohol. Those skilled in the art will appreciate that such cleavage amounts to the hydrolytic cleavage of a bioerodible moiety. Accordingly, the drug (D) may also be described as (a) being coupled to the linking group (Z) via a bioerodible moiety, or (b) forming together with the linking group (Z) a bioerodible moiety.

As referred to herein, the linking group “Z” is a bond or a group which is generally divalent and that couples the prostaglandin drug moiety D to the polymer backbone. As outlined above, the covalent bond between the linking group (Z) and the drug (D) is cleavable so that the drug is releasable.

A part or the whole of the Z group can form part of an ester, an anhydride or a carbonate linkage group. The skilled worker will recognise that each of these linkage groups comprises a covalent bond that is capable of being cleaved (for example hydrolytically and/or enzymatically). Generally, such linkage groups will comprise a covalent bond that is capable of being cleaved hydrolytically so as to release the drug.

At the very least the prostaglandin drug will be releasable from the Z group of the polymer conjugate per se. When the polymer-drug conjugate is bioerodible, the polymer may also bioerode in vivo or in vitro such that the polymer backbone fragments, with the prostaglandin drug moiety remaining tethered to such a fragment(s) via the Z group or even just to a lone Z group as the fragment. In that case, the prostaglandin drug will nevertheless still be capable of being released or cleaved from the Z group, which may or may not still be associated with the polymer conjugate per se.

In the moieties of formulae (I), the prostaglandin drug (D) is coupled to R through a linking group denoted by Z. As used herein, the term “linking group” as used in connection with the group “Z” refers to a group which is generally divalent and that couples D to R. As outlined above, the covalent bond between the linking group (Z) and the prostaglandin drug (D) is cleavable so that the drug is releasable.

In some embodiments the prostaglandin drugs (denoted D in formulae described herein) are conjugated to the polymer backbone via R1 by an ester or anhydride linking group. The drug is therefore covalently linked to Z to form part of an ester linkage (ester bond) or an anhydride linkage (anhydride bond). In this regard, Z therefore comprises the alcohol residue of the ester or anhydride linkage.

In some embodiments, when the polymer-drug conjugate comprises prostaglandin drugs (D) of formula (XX) conjugated to the polymer backbone at R¹ via an ester or anhydride linking group, the polymer-drug conjugate may comprise a moiety of formula (Id) as a part of the polymer backbone:

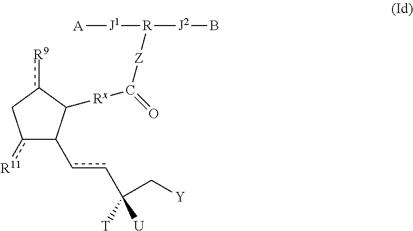

In some embodiments the prostaglandin drugs (denoted D in formulae described herein) are conjugated to the polymer backbone via one of R⁹, R¹¹ and R¹⁵ by an ester or carbonate linking group. The drug is therefore covalently linked to Z to form part of an ester linkage (ester bond) or an carbonate linkage (carbonate bond). In this regard, Z comprises the acid residue of the ester or carbonate linkage.

In some embodiments, when the polymer-drug conjugate comprises prostaglandin drugs (D) of formula (XX) conjugated to the polymer backbone at R⁹ via an ester or carbonate linking group, the polymer-drug conjugate may comprise a moiety of formula (Ie) as a part of the polymer backbone:

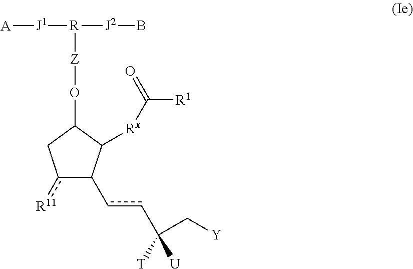

In some embodiments, when the polymer-drug conjugate comprises prostaglandin drugs (D) of formula (XX) conjugated to the polymer backbone at R¹¹ via an ester or carbonate linking group, the polymer-drug conjugate may comprise a moiety of formula (If) as a part of the polymer backbone:

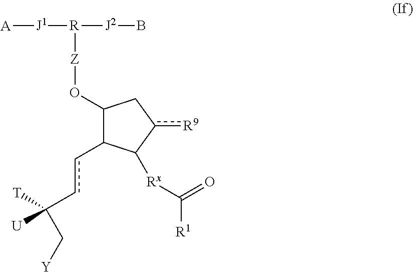

In some embodiments, when the polymer-drug conjugate comprises prostaglandin drugs (D) of formula (XX) conjugated to the polymer backbone at R¹⁵ via an ester or carbonate linking group, the polymer-drug conjugate may comprise a moiety of formula (Ig) as a part of the polymer backbone:

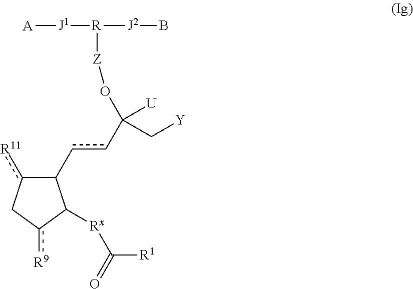

The use of a linking group (Z) can provide facile coupling of the ester or anhydride linked drug to R. It may provide the skilled worker with the ability to couple the ester or anhydride linked drug at a sterically hindered position that could not otherwise be achieved by direct coupling of the drug to R.

Some specific examples of the linking group Z include: —O—; —(O)C—O—; and optionally substituted: —OC(O)—R²—(O)CO—; —C(O)O—R²—(O)CO—; —O—R²—(O)CO—; —C(O)—R²—(O)CO—; —NR^{a}C(O)O—R²—(O)CO—; —OC(O)NR^{a}—R²—(O)CO—; —NR^{a}C(O)—R²—(O)CO—; —C(O)NR^{a}—R²—(O)CO—; —C(O)O—R²—O—; —OC(O)—R²—O—; —O—R²—O—; —C(O)—R²—O—; NR^{a}C(O)O—R²—O—; —OC(O)NR—R²—O—; —NR^{a}C(O)—R²—O—; and —C(O)NR^{a}—R²—O—; where R² represents an optionally substituted hydrocarbyl or optionally substituted heterohydrocarbyl, and R^{a} is H or C1-C6 alkyl. Suitable hydrocarbyl and heterocarbyl may comprise aliphatic, alicyclic or aromatic groups or combinations thereof, and in the case of heterocarbyl group, will also comprise at least one heteroatom selected from the group consisting of N, O and S.

In some embodiments of a polymer-drug conjugate of the invention,

(a) the group D is a prostaglandin drug of formula (XX), wherein R¹ is the acid residue of an ester or anhydride linking group and Z is of a formula selected from the group consisting of:
(i) (R) —O— (D);
(ii) (R) -Q-Ar—O— (D);
(iii) (R) -Q-C₁₋₁₂alkylene-O— (D);
(iv) (R) -Q-Ar-Q-C₁-C₁₂alkylene-O-(D);
(v) (R) -Q-C₁-C₁₂alkylene-Q-Ar—O (D);
(vi) (R) -Q-C₁-C₁₂alkylene-Q-Ar-Q-C₁-C₁₂alkylene-O— (D);
(vii) (R) —OC(O)— (D);
(Viii) (R) -Q-Ar—OC(O)— (D); and
(ix) (R) -Q-C₁₋₁₂alkylene-OC(O)— (D).
   (b) the group D is the prostaglandin drug of formula (XX) wherein one of R⁹, R¹¹ and R¹⁵ is the hydroxy residue (—O—) of an ester or carbonate linking group and Z is of formula selected from the group consisting of
(i) (R) —C(O)(D);
(ii) (R) —OC(O)— (D);
(ii) (R) -Q-Ar—C(O)— (D);
(iii) (R) -Q-C₁₋₁₂alkylene-C(O)— (D);
(iv) (R) -Q-Ar-Q-C₁-C₁₂alkylene-C(O)— (D);
(v) (R) -Q-Ar-Q-C₁-C₁₂alkylene-OC(O)— (D);
(vi) (R) -Q-C₁-C₁₂alkylene-Q-Ar—C(O) (D); and
(vii) (R) -Q-C₁-C₁₂alkylene-Q-Ar-Q-C₁-C₁₂alkylene-C(O)— (D);
   wherein:
(R) indicates the end of the linking group bonded to the R group and (D) indicates the end of the linking group bonded to the prostaglandin drug D;
Ar is optionally substituted aromatic or heteroaromatic hydrocarbon; and
Q is selected from the group consisting of —O—, —C(O)—, —O—C(O)—, —C(O)—O—, —C(O)OC(O)—, —C(O)NR^{a}C(O)—, —OC(O)NR^{a}—, —NR^{a}C(O)O—, —NR^{a}—, —NR^{a}C(O)NR^{a}, —NRaC(O)—, —C(O)NR^{a}—, —S—, —O—C(S)—, —C(S)—O—, —S—C(O)—, —C(O)—S—, —NRC(S)—, and —C(S)NR^{a}—, where R^{a} is hydrogen or C₁ to C₆ alkyl.

The terms “aromatic hydrocarbon” and “heteroaromatic hydrocarbon” in connection with the group “Ar” denotes any ring system comprising at least one aromatic or heteroaromatic ring.

The aromatic hydrocarbon or heteroaromatic hydrocarbon may be optionally substituted by one or more optional substituents as described herein.

The aromatic hydrocarbon or heteroaromatic hydrocarbon may comprise a suitable number of ring members. In some embodiments, the aromatic hydrocarbon or heteroaromatic hydrocarbon comprises from 5 to 12 ring members. The term “ring members” denotes the atoms forming part of the ring system. In an aryl group, the ring atoms are each carbon. In a heteroaromatic hydrocarbon group one or more of the rings atoms are heteroatoms.

Examples of heteroatoms are O, N, S, P and Se, particularly O, N and S. When two or more heteroatoms are present in a heteroaromatic hydrocarbon group, the heteroatoms may be the same or different at each occurrence.

Suitable aromatic hydrocarbon may be selected from the group consisting of phenyl, biphenyl, naphthyl, tetrahydronaphthyl, idenyl, azulenyl, and the like.

Suitable heteroaromatic hydrocarbon may be selected from the group consisting of furanyl, thiophenyl, 2H-pyrrolyl, pyrrolinyl, oxazolinyl, thiazolinyl, indolinyl, imidazolidinyl, imidazolinyl, pyrazolyl, pyrazolinyl, isoxazolidinyl, isothiazolinyl, oxadiazolinyl, triazolinyl, thiadiazolinyl, tetrazolinyl, pyridinyl, pyridazinyl, pyrimidinyl, pyrazinyl, triazenyl, indolyl, isoindolinyl, benzimidazolyl, benzoxazolyl, quinolinyl, isoquinolinyl, and the like.

In some embodiments of the invention, Ar is an optionally substituted C5-12 aromatic hydrocarbon. In some embodiments Ar is optionally substituted phenyl (C6 aromatic hydrocarbon). In some specific embodiments, Ar is para or meta substituted phenyl.

In some embodiments of a polymer-drug conjugate of the invention, when D is linked via R¹ to the polymer backbone, then Z is of a formula selected from the group consisting of:
(R) —O— (D);
(R) —OC(O)—Ar—O— (D);
(R) —NHC(O)—Ar—O— (D);
(R) —C(O)O—C₁₋₁₂alkylene-O— (D);
(R) —OC(O)—C₁₋₁₂alkylene-O— (D).
(R) —OC(O)— (D);
(R) —OC(O)—Ar—OC(O)— (D);
(R) —NHC(O)—Ar—OC(O)— (D);
(R) —C(O)O—C₁-C₁₂alkylene-OC(O)— (D);
(R) —OC(O)—C₁-C₂alkylene-OC(O)— (D).

In one embodiment, when D is linked via R¹ to the polymer backbone, then Z is —O—.

In some embodiments of a polymer-drug conjugate of the invention, when D is linked via one of R⁹, R¹¹ and R¹⁵ to the polymer backbone, then Z is of a formula selected from the group consisting of:
(R) —C(O) (D);
(R) —OC(O)— (D);
(R) —OC(O)—C₁₋₁₂alkylene-C(O)— (D);
(R) —NHC(O)—C₁₋₁₂alkylene-C(O)— (D);
(R) —OC(O)—C₁₋₁₂alkylene-OC(O)— (D);
(R) —NHC(O)—C₁₋₁₂alkylene-OC(O)— (D).

In a specific embodiment, when D is linked via one of R⁹, R¹¹ and R¹⁵ to the polymer backbone, then Z is —C(O)—.

In some embodiments of the present invention, D as shown in formulae described herein is selected from the following group:

| Drug | 1-COOH | 9-OH | 11-OH | 15-OH |
|---|---|---|---|---|
| PGF_{2α} | 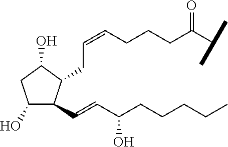 | 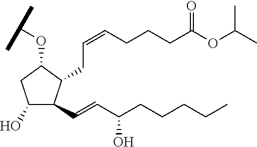 | 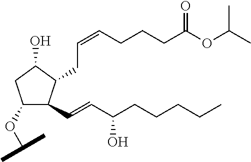 | 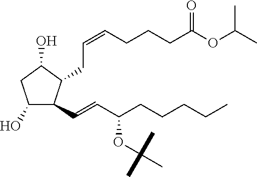 |
| Latanoprost | 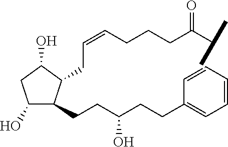 | 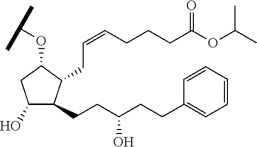 | 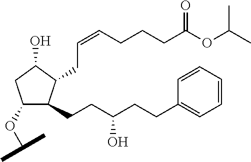 | 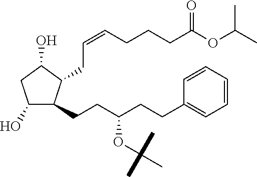 |
| Bimatoprost | 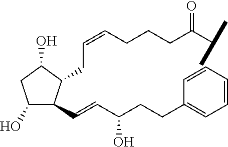 | 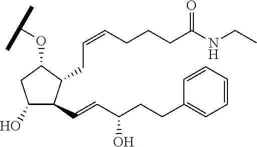 | 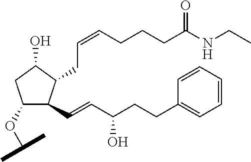 | 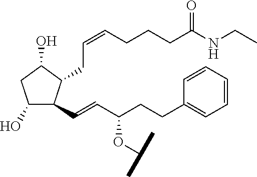 |
| Travoprost | 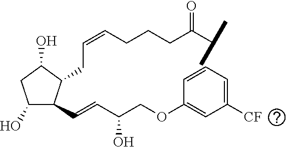 | 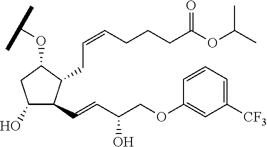 | 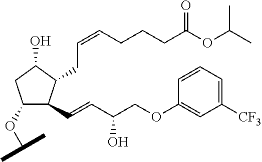 | 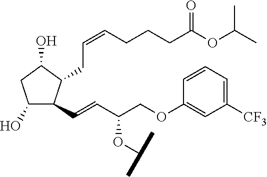 |
| Tafluprost | 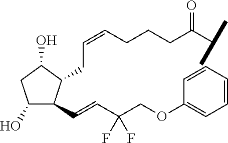 | 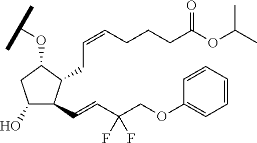 | 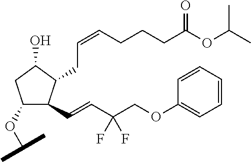 | |
| Unoprostone | 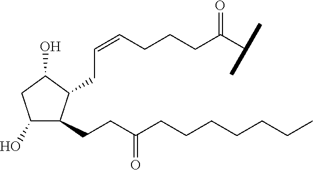 | 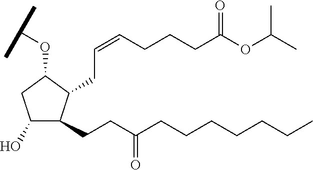 | 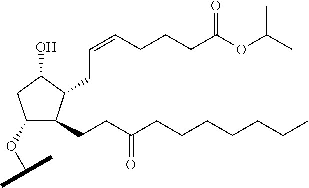 | |

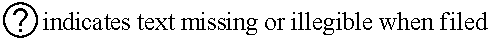

The moiety “R” present in the formulae described herein represents an optionally substituted hydrocarbon. In some embodiments the hydrocarbon may comprise from 1 to 12 carbon atoms, for example from 1 to 10 carbon atoms, from 2 to 8 carbon atoms, or from 3 to 6 carbon atoms. The hydrocarbon may be partially or completely saturated or unsaturated, linear or branched aliphatic, cyclic or aromatic.

In one embodiment, R is an optionally substituted linear or branched hydrocarbon of from 1 to 12 carbon atoms.

R may be optionally substituted with a substituent group. In some embodiments, R is optionally substituted with from 1 to 4 substituent groups selected from the group consisting of hydroxy, amino and carboxylic acid groups. In one form, R is optionally substituted with from 1 to 3 hydroxy groups.

Specific examples of R include a moiety having any one of the following structures:

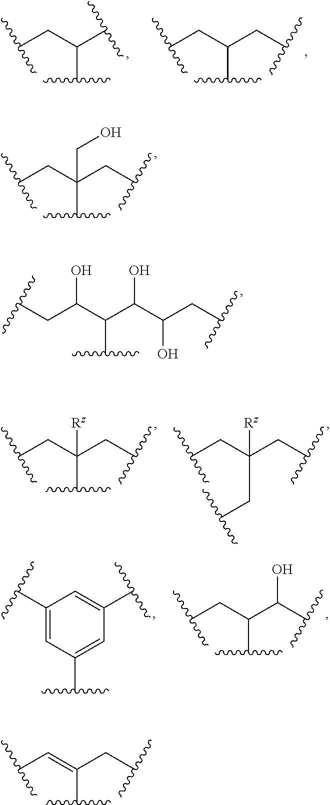

where R^{z} is C₁₋₆alkyl, preferably methyl or ethyl.

The present invention further provides a polymer drug conjugate according to any one of the embodiments described herein, wherein the polymer drug conjugate is a polymer of a monomer of formula (Va):

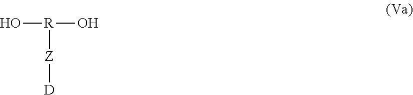

wherein R, Z and D are as hereinbefore defined.

In its broadest aspect, the polymer back polymerpolymer-drug conjugates of the invention may comprise a natural polymer, a synthetic polymer, or a combination thereof.

The polymer backbone may comprise a polymer prepared by a process selected from the group consisting of free radical polymerisation, ionic polymerisation, condensation polymerisation, ring-opening polymerisation, and combinations thereof.

The polymer backbone may comprise a homopolymer or a copolymer, for example, a random copolymer or a block copolymer.

The polymer backbone may comprise a polymer of any suitable architecture. In specific embodiments of the invention, the polymer backbone comprises a linear polymer.

Suitable polymer backbones may comprise a polymer selected from the group consisting of vinyl polymers, acrylic polymers, methacrylic polymers, polyether polymers, polyester polymers, polyanhydride polymers, polycarbonate polymers, polyamide polymers, polyimide polymers, polyurethane polymers, polyurea polymers, polysiloxane polymers, fluoropolymers, polysaccharides, polypeptides, polynucleic acids, copolymers thereof, and combinations thereof. Such polymers may be prepared by polymerising at least one monomer selected from the group consisting of vinyl monomers, polyfunctional monomers and cyclic monomers.

The polymer backbone may be selected to be compatible with a pre-selected environment, for an example, a biological environment.

In embodiments of the invention the polymer-drug conjugate is bioerodible and the polymer backbone comprises a bioerodible polymer. At least a portion of the polymer backbone comprises a bioerodible polymer. In some embodiments, other types of polymer may optionally be present in the polymer backbone in addition to the bioerodible polymer.

In some embodiments, the entire polymer backbone is bioerodible. Accordingly, in some embodiments the polymer backbone of polymer-drug conjugates in accordance with the invention includes moieties that are “bioerodible”.

By being “bioerodible” is meant that the moieties in the conjugates have a molecular structure that is susceptible to break down (i.e. a reduction in molecular weight) by chemical or enzymatic decomposition in a biological environment (e.g. within a subject or in contact with biological material such as blood, tissue etc), as opposed to physical degradation. Such decomposition will typically be via the hydrolysis of labile moieties that form part of the molecular structure of the conjugates. In other words, the conjugates will comprise moieties that are susceptible to hydrolytic cleavage. The rate of hydrolysis of the biodegradable moieties may vary over time, or be activated by any number of extrinsic or intrinsic factors (e.g. light, heat, radiation, pH, enzymatic or non-enzymatic cleavage, etc.).

Polymer backbones employed in polymer-drug conjugates of the invention may also be biocompatible. As used herein, “biocompatible polymer” refers to a polymer that both in its intact, that is, as synthesized state and in its decomposed state (i.e. its degradation products), is compatible with living tissue in that it is not, or at least is minimally, toxic to living tissue; does not, or at least minimally and reparably does, injure living tissue; and/or does not, or at least minimally and/or controllably does, cause an immunological reaction in living tissue.

In embodiments of a bioerodible polymer-drug conjugate comprising a moiety of formula (I), the bioerodible polymer forms at least a part of A and/or B. As used herein the term “at least a part” is intended to signify that at least a portion of A and/or B be composed of a bioerodible polymer. Other types of polymer may optionally be present in A and/or B in addition to the bioerodible polymer. In some embodiments of a bioerodible polymer-drug conjugate comprising a moiety of formula (I), A and B are each entirely composed of bioerodible polymer.

In embodiments of a polymer-drug conjugate of the invention, the conjugate comprises as part of its polymer backbone a moiety of general formula (Ic):

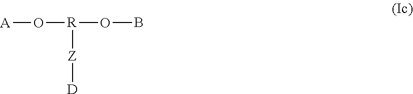

where A and B, which may be the same or different, represent the remainder of a bioerodible polymer backbone.

A and B in formulae described herein may be selected from or comprise a range of materials including: polyurethanes; polyurethanes optionally comprising one or more chain extenders (e.g. polyester); polyesters (e.g. PLGA (poly(lactic-co-glycolic acid)), PLA (polylactic acid), PGA (polyglycolic acid), PHB (polyhydroxybutyrate), PCL (polycaprolactone); polyamides; polyanhydrides, polycarbonates; polyimides; and combinations thereof. In some embodiments, A and B are selected from or comprise: polyurethanes; polyesters; polyanhydrides; polyamides and combinations thereof. A and/or B will also generally comprise one or more drug moieties covalently bonded to the polymer backbone.

Depending upon the intended application, A and B may be selected for their biocompatible and/or their bioerodible properties. Those skilled in the art can readily select polymers to provide for such properties.

In some embodiments, A and B may be selected from or comprise a polyester. In that case, the monomeric units that are polymerised to form the polyester, typically a diacid and a diol, will each be coupled via a biodegradable ester moiety.

In some embodiments, A and B may be selected from or comprise a polyurethane. In that case, the monomeric units that are polymerised to form the polyurethane, typically a diisocyanate and a diol, will each be coupled via a biodegradable urethane moiety. The urethane moiety may be less labile than an ester, anhydride or carbonate moiety. As a result, a polymer backbone that comprises or is composed of a polyurethane may erode at a rate that is slower than the rate of cleavage of the ester, anhydride or carbonate linkage coupling the prostaglandin drug to the polymer backbone. As a result, a prostaglandin drug conjugated to a polyurethane polymer backbone may advantageously be released from the polymer conjugate before substantial erosion of the polymer backbone occurs.

In some embodiments, A and B may be selected from or comprise a copolymer of polyurethane and polyester. In that case, the biodegradable polymer of A and/or B may be a poly(urethane-ester) or a poly(ester-urethane) formed by polymerising a diisocyanate with a polyester macromonomer or macromer. The polyester macromer will be formed from monomeric units that are coupled via a biodegradable moiety (as discussed above), and the polymerisation of it with the diisocyanate will give rise to the poly(urethane-ester) having monomeric units that are all coupled via a biodegradable urethane or ester moiety. The biodegradable polymer of A and/or B may also be a poly(ester-urethane) formed by polymerising a ester containing monomer or macromonomer with a polyurethane macromonomer or macromer. In that case, the polyurethane macromer will be formed from monomeric units that are coupled via a biodegradable moiety (as discussed above), and the polymerisation of it with the ester monomer or macromonomer will give rise to the poly(ester-urethane) having monomeric units that are all coupled via a biodegradable urethane or ester moiety.

In some embodiments, A and B may be selected from or comprise a copolymer of polyurethane and polyether. In that case, the biodegradable polymer of A and/or B may be a poly(urethane-ether) or a poly(ether-urethane) formed by polymerising a diisocyanate with a polyether macromonomer or macromer. The polyether macromer will be formed from monomeric units that are coupled via a biodegradable moiety (as discussed above), and the polymerisation of it with the diisocyanate will give rise to the poly(urethane-ether) having monomeric units that are all coupled via a biodegradable urethane or ether moiety. The biodegradable polymer of A and/or B may also be a poly(ether-urethane) formed by polymerising a ether containing monomer or macromonomer with a polyurethane macromonomer or macromer. In that case, the polyurethane macromer will be formed from monomeric units that are coupled via a biodegradable moiety (as discussed above), and the polymerisation of it with the ether monomer or macromonomer will give rise to the poly(ether-urethane) having monomeric units that are all coupled via a biodegradable urethane moiety.

Polymer-drug conjugates of the invention can be advantageously altered to incorporate other monomers or components to provide appropriate polymer properties to suit a particular application (e.g. flexibility, structural strength, rate of release of prostaglandin drug). The physical properties of the material can be altered through changing the composition of the polymer backbone, for example, as represented by A and B in formula (I).

Polymer-drug conjugates as described herein may optionally comprise a hydrophilic group. In one aspect of the invention, polymer-drug conjugates as described herein comprise a hydrophilic group in the polymer backbone. In some embodiments, the hydrophilic group may comprise at least one active-hydrogen group. The hydrophilic group may be provided by or derived from a monomer comprising at least one active-hydrogen containing group. As used herein, the term “active-hydrogen containing group” refers to a group comprising one or more hydrogen atoms that are capable of participating in hydrogen bonding interactions. Groups containing active-hydrogen atoms include for example, hydroxy, amine and carboxylic acid. Monomers containing an active-hydrogen group may comprise a single active-hydrogen group, it they may comprise a plurality of active-hydrogen groups. For example, a macromonomer may comprise a plurality of active-hydrogen groups.

Hydrophilic groups may increase the hydrophilicity of polymer-drug conjugates of the invention, for example, by promoting hydrogen bonding interactions with an aqueous environment. The polymer backbone within the conjugate may exhibit hydrophilic character.

Increasing the hydrophilicity of the polymer-drug conjugate may advantageously help promote efficient drug release.

By “hydrophilic” is meant that a substance, component or group as described herein has an affinity for water, or contains groups that will attract water its structure. A hydrophilic substance, component or group will generally be soluble in water or miscible with water.

Solubility may be determined by reference to texts such as The International Pharmacopoeia, Fourth Edition, 2006. A hydrophilic substance, component or group may possess a solubility of 1 gram (g) of solid in up to 30 millilitres (ml) of aqueous solvent (water) at 20° C.

When present, the hydrophilic group may constitute at least about 5 mol %, at least about 10 mol %, or at least about 15 mol % of the polymer-drug conjugate.

In some embodiments of a polymer-drug conjugate comprising a moiety of formula (I) or (Ic) as a part of the polymer backbone, at least one of A and B comprises a hydrophilic group. In some embodiments the hydrophilic group comprises a plurality of active-hydrogen groups.

In some embodiments, at least one of A and B comprises at least one hydrophilic group incorporated in the conjugate as part of the polymer backbone.

In some embodiments, at least one of A and B comprises at least one hydrophilic group covalently attached to and pendant from the polymer backbone. In such embodiments, the polymer-drug conjugate contains at least one pendant hydrophilic group and pendant drug moieties attached to the polymer backbone.

In some embodiments, A and/or B may comprise a combination of pendant and intrα-chain incorporated hydrophilic groups.

In polymer-drug conjugates comprising a moiety of formula (I) or (Ic) as a part of its backbone, at least one of A and B comprise may a hydrophilic group. The hydrophilic group may be present in A and/or B in combination with a polymer, for example, a biodegradable polymer.

In some embodiments, the hydrophilic group may comprise an oligomer or polymer derived from one or more monomers comprising a plurality of active-hydrogen groups, wherein the active-hydrogen groups are selected from the group consisting of hydroxy, amine, carboxylic acid, and combinations thereof.

In some embodiments, the active-hydrogen containing monomer comprises at least one selected from the group consisting of poly(ethylene glycol), poly(lactic acid-co-glycolic acid) (PLGA), poly(1,5-dioxepan-2-one) (PDOO), poly(glycerol acetate) (PGAc), poly(hydroxy butyrate),), poly(glycerol phosphate), an amino acid polymer (such as polylysine, polyglutamic acid, etc), an amino acid oligomer, low molecular weight diols (for example C2-C4 diols, such as ethylene glycol, propane diol, propylene glycol, butane diol etc), amino acids (lysine, glutamic acid etc), lactic acid, glycolic acid, hydroxy acids (for example, hydroxybutyric acid etc), 1,5-dioxepan-2-one, glycerol acetate, glycerol phosphate, or combinations thereof, or copolymers thereof.

The active-hydrogen containing monomer may be a macromonomer comprising an oligomeric or polymeric moiety selected from the group consisting of poly(ethylene glycol), poly(lactic acid-co-glycolic acid) (PLGA), poly(1,5-dioxepan-2-one) (PDOO), poly(glycerol acetate) (PGAc), poly(hydroxy butyrate), poly(glycerol phosphate), an amino acid polymer (such as polylysine, polyglutamic acid, etc), or an amino acid oligomer, or combination of, or a copolymer of, such polymeric or oligomeric moieties. For example, a macromonomer may comprise a combination of poly(ethylene glycol) and PLGA.

Macromonomers comprising an oligomeric or polymeric moiety will generally comprise a plurality of active hydrogen groups. Oligomeric or polymeric moieties present in a macromonomer may or may not be bioerodible.

The incorporation of hydrophilic groups comprising oligomers or polymers such as polylactic-co-glycolic acid (PLGA), and amino acid polymers (such as polylysine, polyglutamic acid, etc) and amino acid oligomers in the polymer backbone of polymer-drug conjugates of the invention may be advantageous as such oligomers and polymers are also formed from monomeric units coupled via biodegradable moieties, such as ester and amide moieties. As a result, a fully bioerodible polymer-drug conjugate may be produced. Such fully bioerodible conjugates may be particularly suitable for use in implants.

One skilled in the art would appreciate that hydrophilic groups comprising polymers such as poly(ethylene glycol) may not be bioerodible as the monomeric (i.e. diol) units of the poly(ethylene glycol) are coupled via ether moieties which are not bioerodible. However, such groups are generally biocompatible.

In some embodiments A and B independently comprise a polymer selected from the group consisting of polyurethanes, polyesters, poly(urethane-ethers), poly(ester-ethers), poly(urethane-esters), and poly(ester-urethanes). The ether or ester component of the poly(urethane-ethers), poly(ester-ethers), poly(urethane-esters) and poly(ester-urethanes) may represent a hydrophilic group.

In some embodiments the ether component comprises at least one selected from the group consisting of poly(ethylene glycol) (PEG) and poly(glycerol acetate). The ether component may have a molecular weight in the range of from about 200 to about 15,000, preferably from about 500 to about 5,000.

In some embodiments the ester component comprises poly(lactide-co-glycolide) (PLGA). The ester component may have a molecular weight in the range of from about 200 to about 15,000, preferably from about 500 to about 5,000. PLGA employed in the invention may comprise lactic acid and glycolic acid at different ratios. The ratio of lactic acid to glycolic acid may be in the range of from 10:90 to 90:10. In general, higher relative amounts of glycolic acid to lactic acid in the PLGA polymer, will provide a more hydrophilic polymer.

In some embodiments the poly(ester-ether) component comprises at least one selected from the group consisting of poly(1,5-dioxepan-2-one) (PDOO). The poly(ester-ether) component may have a molecular weight in the range of from about 200 to about 15,000, preferably from about 500 to about 5,000.

In some embodiments, the polymer-drug conjugate of the invention comprises a polymer backbone comprising a polyurethane polymer formed with a polyisocyanate and optionally one or more monomers comprising a plurality of active-hydrogen groups selected from hydroxy, amine and carboxylic acid.

The present invention also provides a polymer-drug conjugate comprising a polymer backbone and a plurality of prostaglandin drugs conjugated to the polymer backbone, wherein the polymer-drug conjugate is obtained by polymerising a drug-monomer conjugate of formula (V):

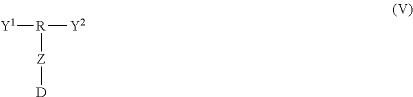

where:
Y¹ and Y² each independently represent a reactive functional group, or Y¹ and Y² together form part of a cyclic group capable of ring-opening;
R is an optionally substituted hydrocarbon;
Z is a linking group;

D is a prostaglandin drug of formula (XX); and
D and Z together form an ester, anhydride or carbonate linking group, with at least one monomer comprising compatible chemical functionality.

The present invention also provides a process for preparing a polymer-drug conjugate comprising as part of its polymer backbone a moiety of general formula (I):

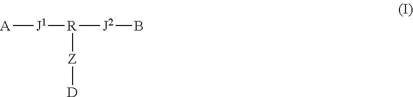

where:
A and B, which may be the same or different, represent the remainder of the polymer backbone and are (i) attached to the -J¹-R(ZD)-J²- moiety as shown in formula (I) via a bioerodible moiety, and (ii) each formed from monomeric units that are coupled via bioerodible moieties;
J¹ and J² are independently selected from the group consisting of oxygen, C(O) and NRₐ where R^{a} is hydrogen or C1 to C6 alkyl;
R is an optionally substituted hydrocarbon;
Z is a linking group;
D is a prostaglandin drug of formula (XX); and
D and Z together form an ester, anhydride or carbonate linking group,
   said process comprising a step of polymerising a drug-monomer conjugate of formula (V):

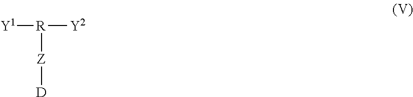

where:
Y¹ and Y² each independently represent a reactive functional group, or Y¹ and Y² together form part of a cyclic group capable of ring-opening; and
R, Z and D are as defined above;
   with at least one monomer comprising compatible chemical functionality.

In accordance with the invention, the drug-monomer conjugate has general formula (V):

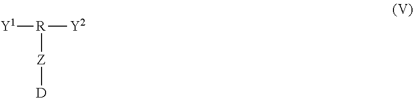

where
Y¹ and Y² each independently represent a reactive functional group, or Y¹ and Y² together form part of a cyclic group capable of ring-opening;
R is an optionally substituted hydrocarbon;
Z is a linking group;
D is a prostaglandin drug of formula (XX); and
D and Z together form an ester, anhydride or carbonate linking group.

In the drug-monomer conjugate of formula (V), the groups R, Z and D may be selected from any one of the groups defined herein.

The groups Y¹ and Y² in drug-monomer conjugates of formula (V) may each independently represent a terminal reactive functional group. In some embodiments, Y¹ and Y² are independently selected from the group consisting hydroxy, isocyanate, anhydride, carboxylic acid, carboxylic acid ester, carboxylic acid halide and amine.

In some embodiments, Y¹ and Y² are each hydroxy. In that case, the drug-monomer conjugate of formula (V) will be a diol having a structure of formula (Va):

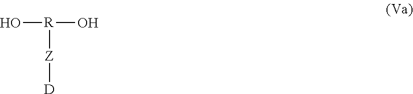

where: R, Z and D are as defined herein.

Examples of a drug-monomer conjugate of formula (Va) a prostaglandin drug of general formula (XX) (D) are shown below:

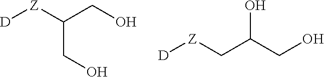

where
Z is a linking group;
D is a prostaglandin drug of formula (XX); and
D and Z together form an ester, anhydride or carbonate linking group.

Examples of a drug-monomer conjugate of formula (Va) that comprise a —O— linking group (Z) and a prostaglandin drug of general formula (XX) (D) are shown below:

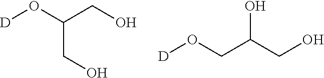

An example of a drug-monomer conjugate of formula (Va) that comprises a —OC(O)C1-12alkylene-C(O)— linking group (Z) and a prostaglandin drug of general formula (XX) (D) is shown below:

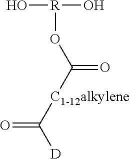

where R represents an optionally substituted hydrocarbon.

The choice of linking group will determine the spacing of the D from the OH groups in the monomers of formula (Va). In this respect, the use of a linking group can provide a means to distance D from the OH groups. This can facilitate polymerisation of the monomers by reducing steric crowding around the OH groups.

In forming the monomer of formula (V), prior to conjugation the prostaglandin drug (denoted by D) necessarily comprises compatible functionality so as to promote coupling of the drug to the monomer through Z.

A part or the whole of the Z group can form part of an ester, an anhydride or a carbonate linkage group. The skilled worker will recognise that each of these linkage groups comprises a covalent bond that is capable of being cleaved (for example hydrolytically, enzymatically and/or by a radical mechanism). Generally, such linkage groups will comprise a covalent bond that is capable of being cleaved hydrolytically so as to release the drug.

Despite the prostaglandin drug being releasable from the monomer of formula (V), it will be appreciated that the intention of the present invention is for the agent to be released after the monomer has been polymerised to form polymer.

In one embodiment, the drug-monomer conjugate of formula (Va) may have a formula of:

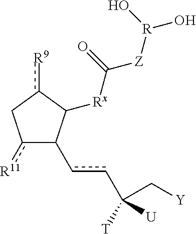

where R^{x}, R⁹, R¹¹, T, U, Y, Z and R are as herein defined.

In one form, the drug-monomer conjugate of may have a formula of:

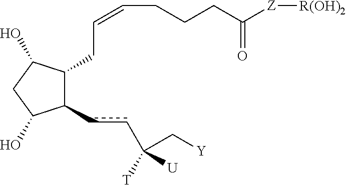

wherein
T and U are each fluoro, or T and U together form oxo, or T is hydroxy and U is hydrogen; and
Z, Y and R are as herein defined.

In such embodiments as shown above, the prostaglandin drug (D) is linked via R¹ to the group Z in the drug-monomer conjugate.

In one embodiment, the drug-monomer conjugate of formula (Va) may have a formula of:

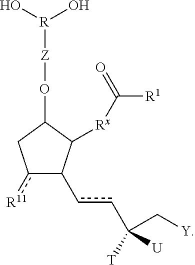

In such embodiments, the prostaglandin drug (D) is linked via R⁹ to the group Z in the drug-monomer conjugate.

In one embodiment, the drug-monomer conjugate of formula (Va) may have a formula of:

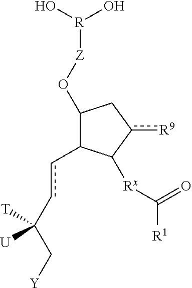

In such embodiments, the prostaglandin drug (D) is linked via R¹¹ to the group Z in the drug-monomer conjugate.

In one embodiment, the drug-monomer conjugate of formula (Va) may have a formula of:

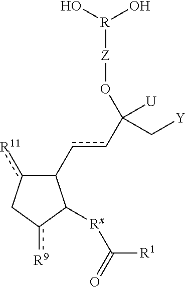

In another form the drug-monomer conjugate may have a formula of:

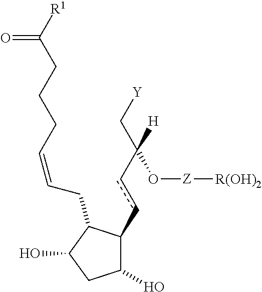

wherein
R¹ is OH, C₁ to C₆ alkoxy or C₁ to C₆ alkylamino (preferably OH, isopropoxy or ethylamino); and
Z, R and Y are as defined.

In such embodiments, the prostaglandin drug (D) is linked via R¹⁵ to the group Z in the drug-monomer conjugate.

In some embodiments, the drug-monomer conjugate of formula (V) may have a more specific structure as shown in the following illustrations:

Based on the free acid form of Latanoprost shown directly below:

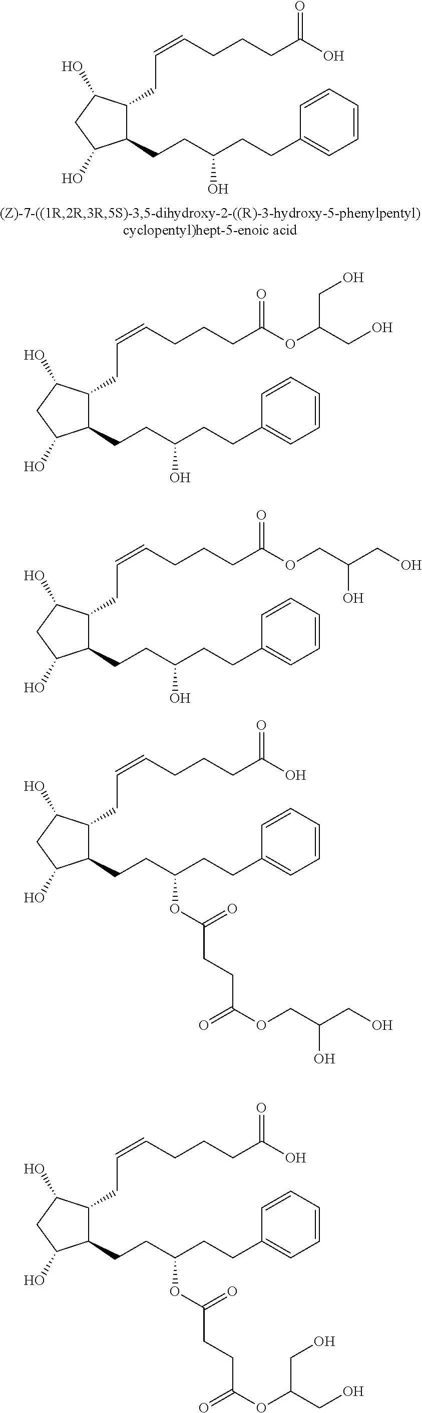 [SMILES: O=C(O)CCC/C=C\C[C@@H]1[C@@H](CC[C@@H](O)CCc2ccccc2)[C@H](O)C[C@@H]1O ; O=C(CCC/C=C\C[C@@H]1[C@@H](CC[C@@H](O)CCc2ccccc2)[C@H](O)C[C@@H]1O)OC(CO)CO ; O=C(CCC/C=C\C[C@@H]1[C@@H](CC[C@@H](O)CCc2ccccc2)[C@H](O)C[C@@H]1O)OCC(O)CO ; O=C(O)CCC/C=C\C[C@@H]1[C@@H](CC[C@H](CCc2ccccc2)OC(=O)CCC(=O)OCC(O)CO)[C@H](O)C[C@@H]1O ; O=C(O)CCC/C=C\C[C@@H]1[C@@H](CC[C@H](CCc2ccccc2)OC(=O)CCC(=O)OC(CO)CO)[C@H](O)C[C@@H]1O]

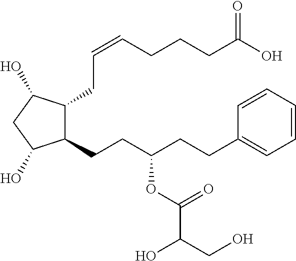 [SMILES: O=C(O)CCC/C=C\C[C@@H]1[C@@H](CC[C@H](CCc2ccccc2)OC(=O)C(O)CO)[C@H](O)C[C@@H]1O]

When the prostaglandin drug (D) is linked to R via an ester linking group at R¹ in the α-chain of the prostaglandin or substituted prostaglandin, the drug-monomer conjugate may have a structure as illustrated in the embodiments shown below:

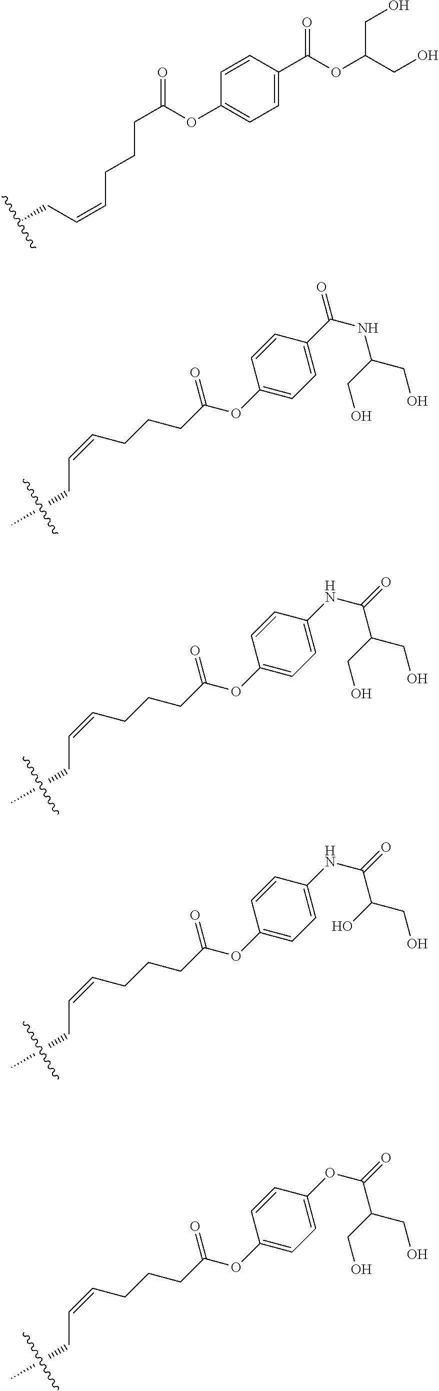

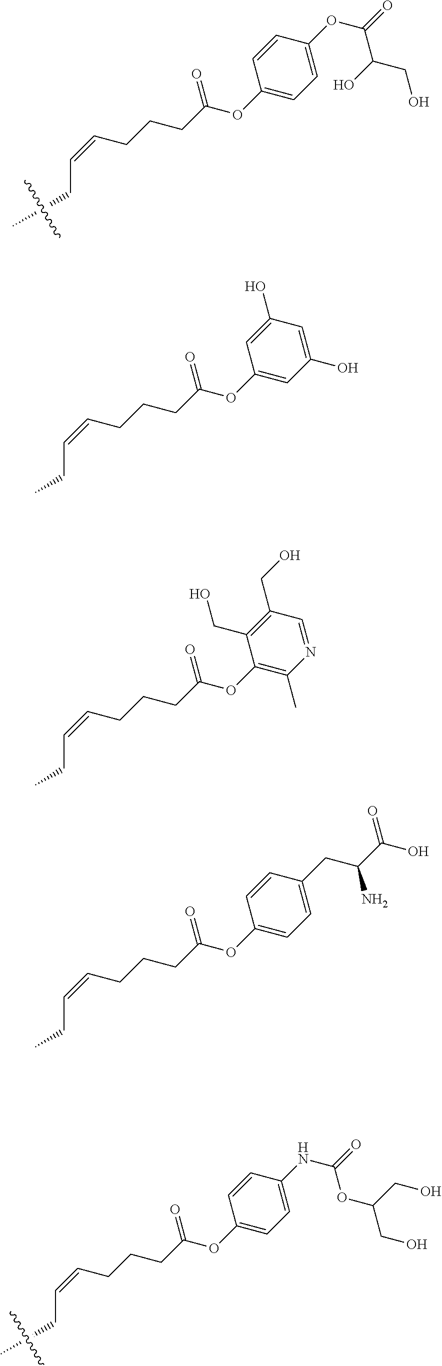

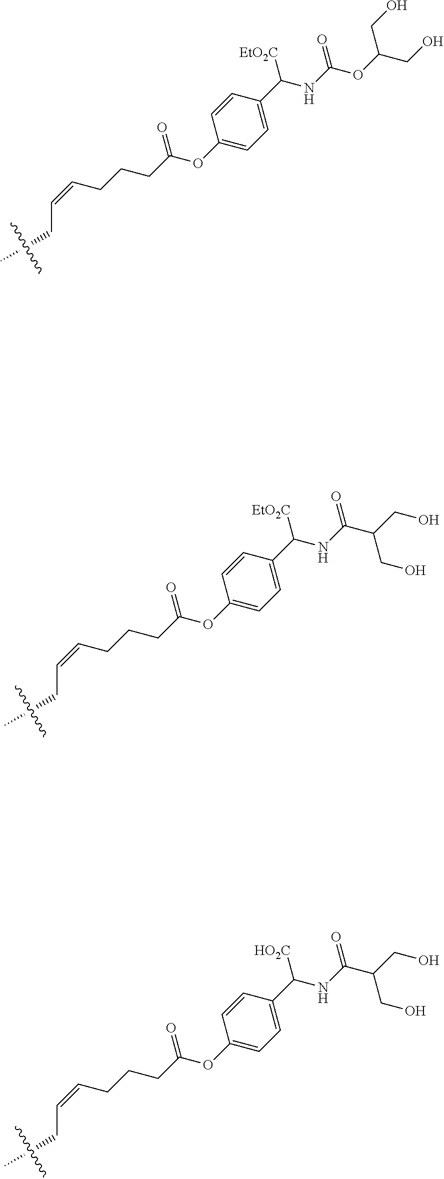

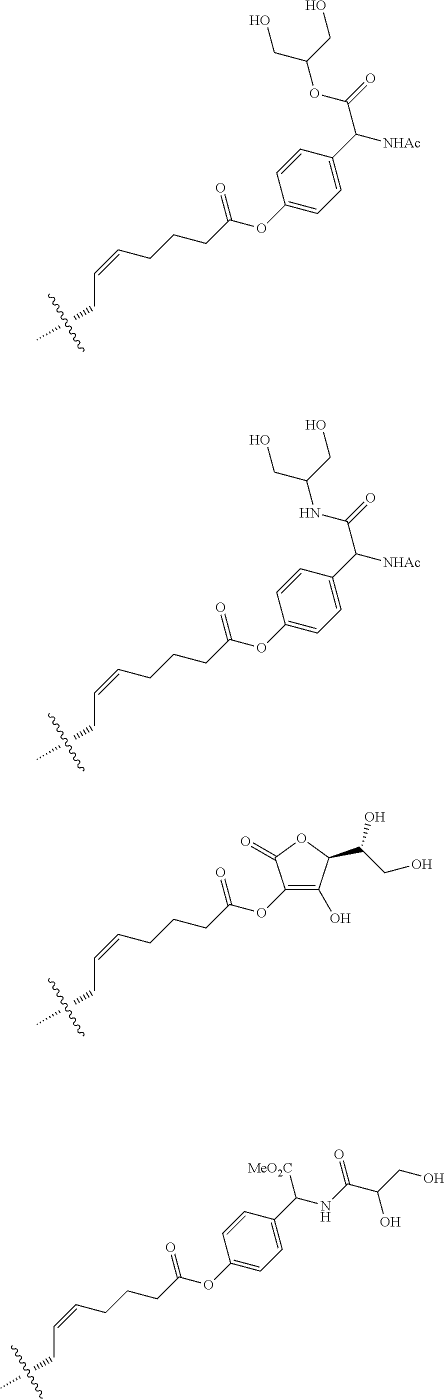

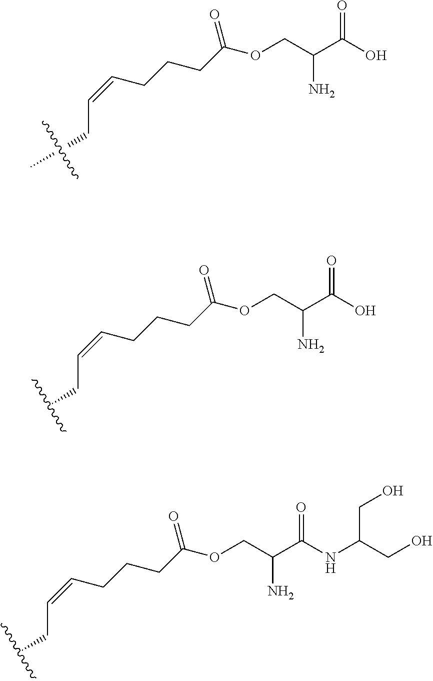

where [symbol] represents where the α-chain is attached to the 5-membered ring of the prostaglandin or substituted prostaglandin.

When the prostaglandin drug (D) is linked to R via an anhydride linking group at R¹ in the α-chain of the prostaglandin or substituted prostaglandin, the drug-monomer conjugate may have a structure as illustrated in the embodiments shown below:

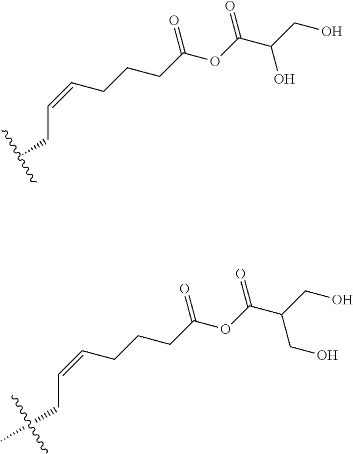

where [symbol] represents where the α-chain is attached to the 5-membered ring of the prostaglandin or substituted prostaglandin.

When the prostaglandin drug (D) is linked to R via an ester linking group at R¹⁵ in the w-chain of the prostaglandin or substituted prostaglandin, the drug-monomer conjugate may have a structure as illustrated in the embodiments shown below:

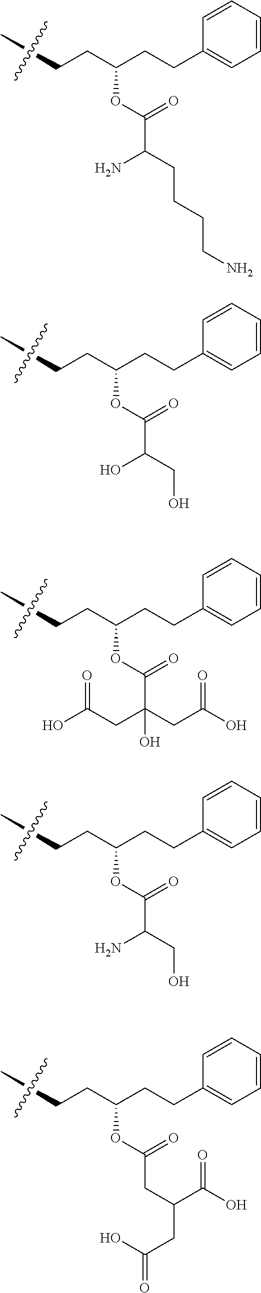

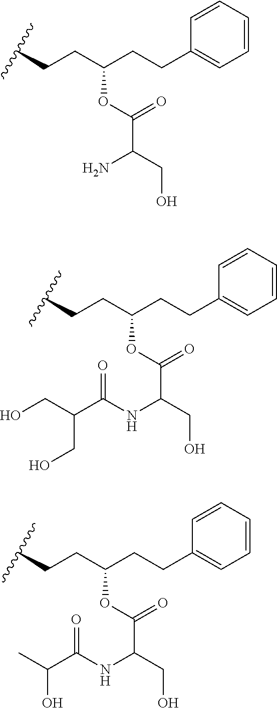

where [symbol] represents where the ω-chain is attached to the 5-membered ring of the prostaglandin or substituted prostaglandin.

When the prostaglandin drug (D) is linked to R via carbonate linking group at R¹⁵ in the ω-chain of the prostaglandin or substituted prostaglandin, the drug-monomer conjugate may have a structure as illustrated in the embodiments shown below:

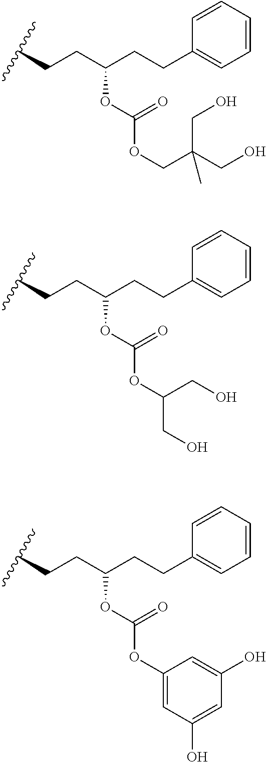

where [symbol] represents where the ω-chain is attached to the 5-membered ring of the prostaglandin or substituted prostaglandin.

One skilled in the art would understand that the above ester and carbonate linking groups at the 15 position of the prostaglandin or substituted prostaglandin, are also able to be formed at the 9 and 11 positions of the prostaglandin or substituted prostaglandin, to provide drug-monomer conjugates wherein D is linked at the 9 or 11 position to R by such linking groups.

Techniques, equipment and reagents well known in the art can advantageously be used to prepare the drug-monomer conjugates in accordance with the invention.

Examples of general strategies for synthesising drug-monomer conjugates of formula (V), which employ protecting group strategies, are represented in Scheme 1 below (where D is as previously defined and D′ is that part of the releasable drug other than the hydroxy or carboxylic acid):

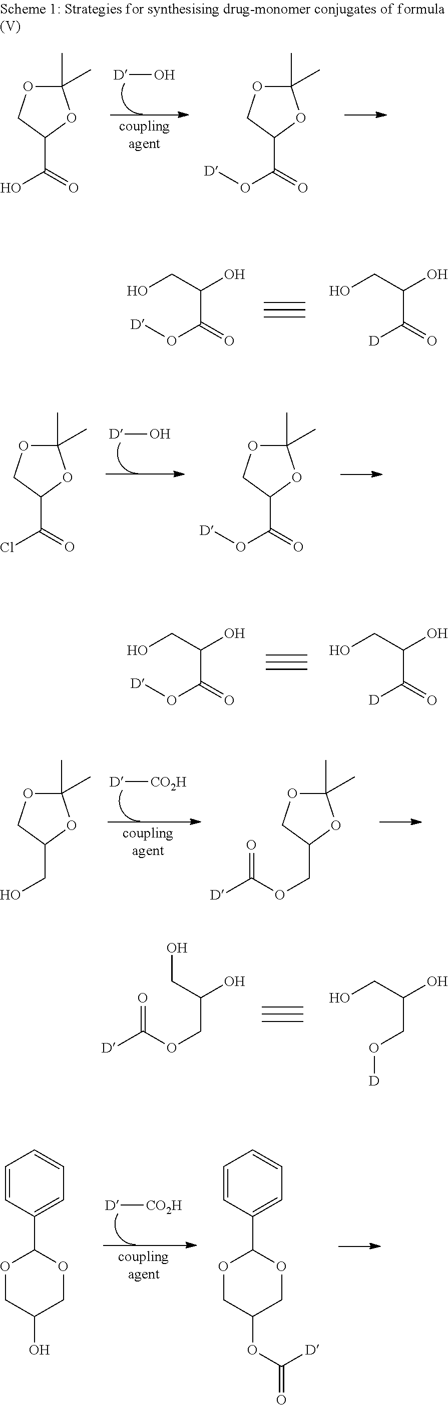

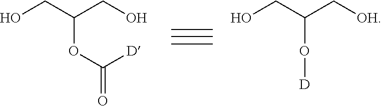

Examples of general strategies for synthesising drug-monomer conjugates of formula (V), which employ protecting group strategies and use diacid-based linking groups, are represented in Scheme 2 below (where p is an integer from e.g. 1 to 12, D is as herein defined; and D′ is that part of the releasable drug other than the hydroxy or carboxylic acid):

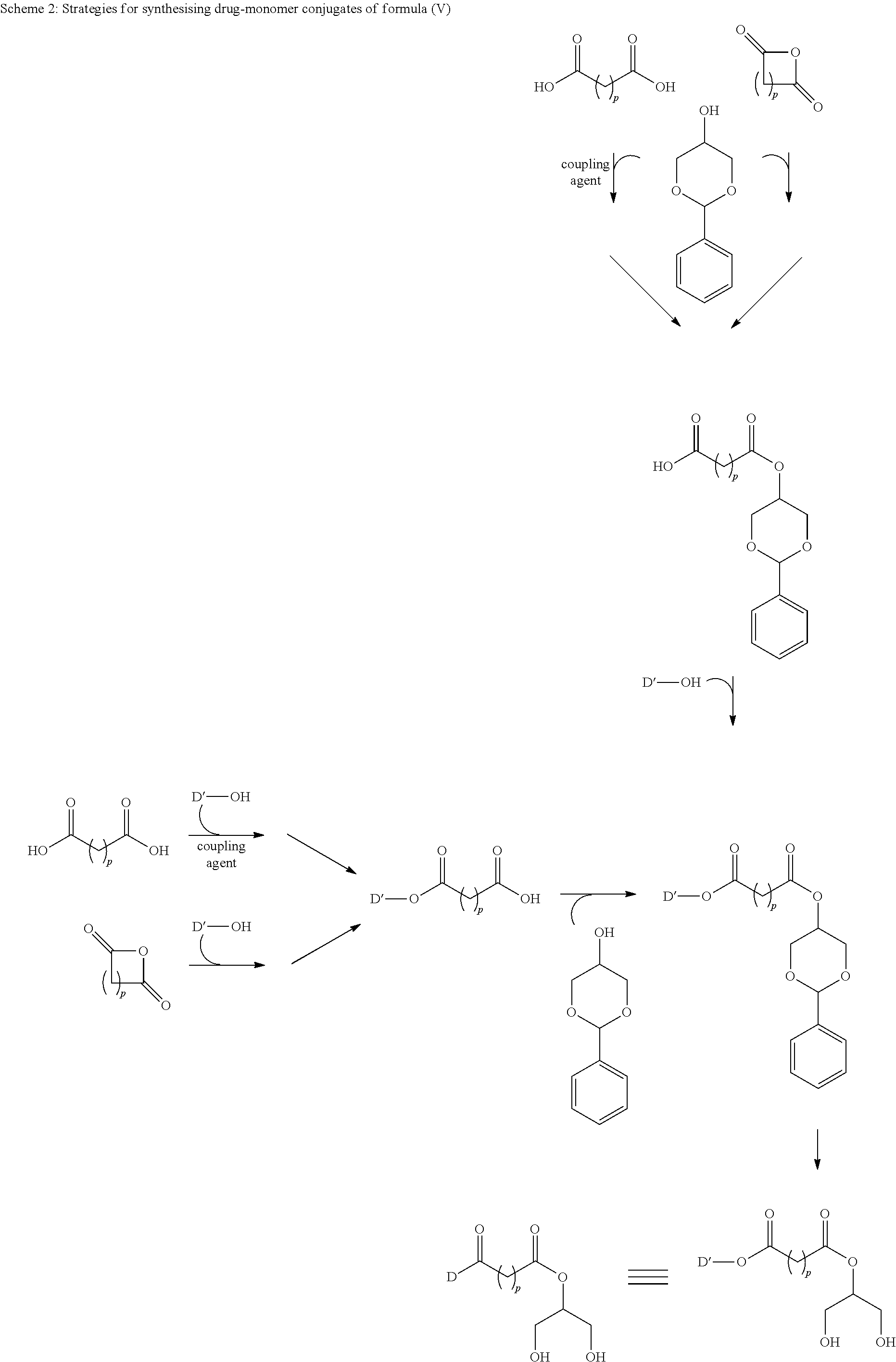

In some embodiments, Y¹ and Y² together with R form part of a cyclic functional group capable of ring-opening. For example, Y¹ and Y² together with R may form part of a cyclic group selected from the group consisting of a cyclic carbonate, a cyclic epoxide, a lactam, a lactone, a cyclic anhydride, and a cyclic carbamate. The cyclic group may contain from 4 to 8 ring members, or from 5 to 7 ring members.

One skilled in the art would appreciate that under suitable polymerisation conditions, a cyclic monomer may undergo ring opening with a monomer comprising compatible chemical functionality to form polymers such as polyesters (from cyclic carbonates and cyclic lactones), polyethers (from cyclic epoxides), polyamides (from lactams), polyanhydrides (from cyclic anhydrides), and polyurethanes (from cyclic carbamates). Such polymers may be homopolymers or copolymers.

Drug-monomer conjugates of formula (V) may be prepared using techniques and methods known in the art.

Drug-monomer conjugates comprising a prostaglandin or substituted prostaglandin linked via an ester linking group at the 1 position may be prepared using a number of different techniques. One technique involves esterification of a prostaglandin or substituted prostaglandin, or transesterification of a prodrug, with a polyol, such as glycerol (a triol). An example is shown below with latanoprost:

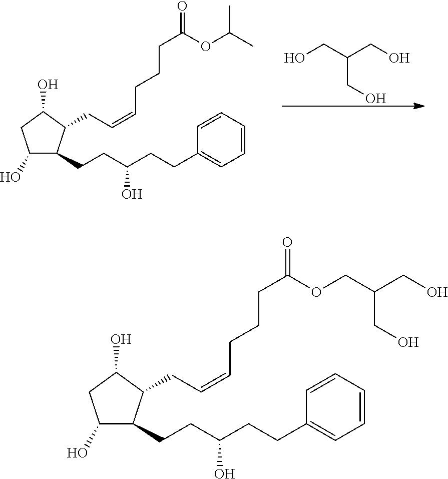 [SMILES: CC(C)OC(=O)CCC/C=C\C[C@@H]1[C@@H](CC[C@@H](O)CCc2ccccc2)[C@H](O)C[C@@H]1O ; OCC(CO)CO ; O=C(CCC/C=C\C[C@@H]1[C@@H](CC[C@@H](O)CCc2ccccc2)[C@H](O)C[C@@H]1O)OCC(CO)CO]

Drug-monomer conjugates comprising a prostaglandin or substituted prostaglandin linked via an ester linking group at the 1 position may also be prepared through the use of appropriate coupling agents to generate the ester linkage. Two examples are shown below:

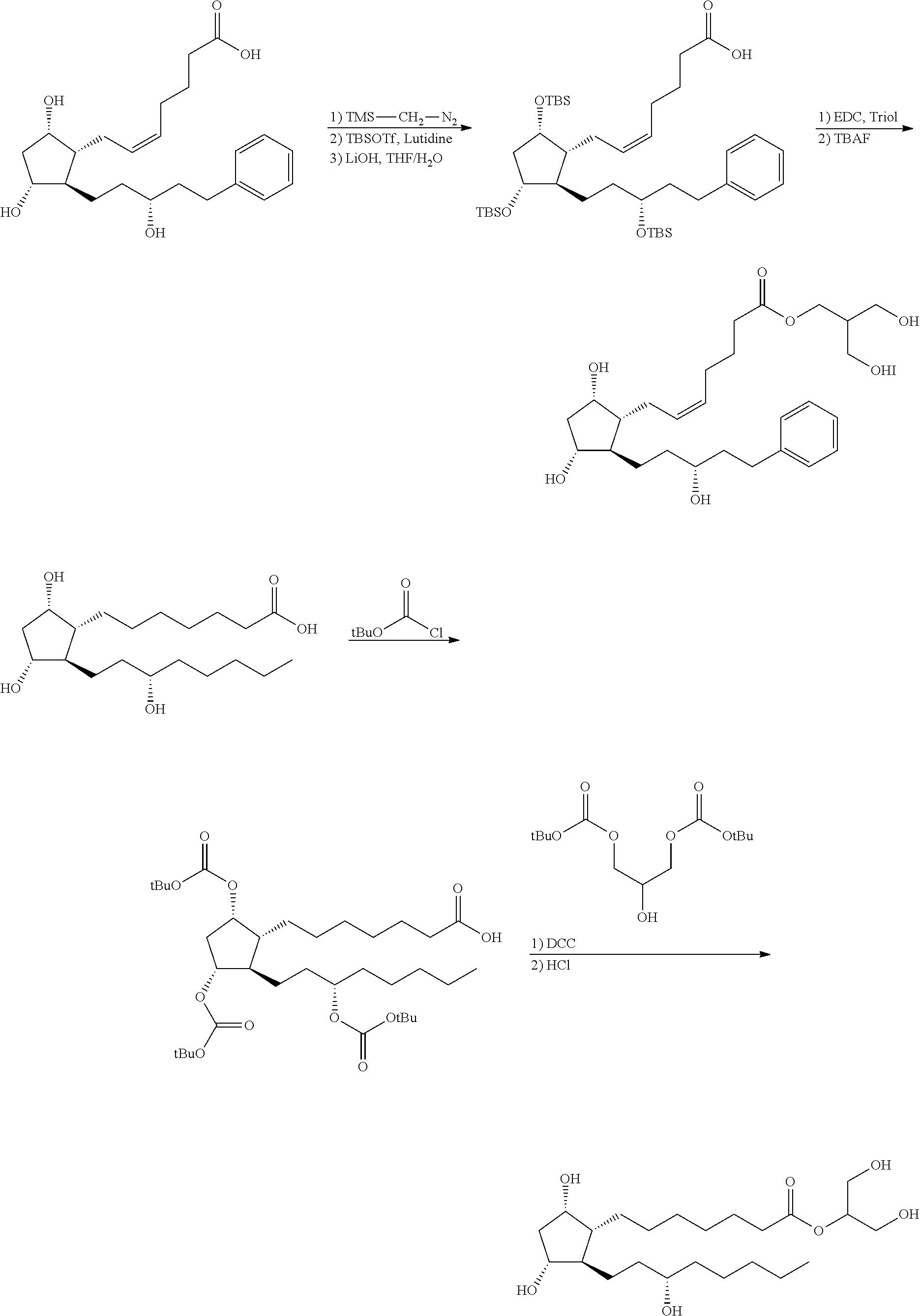

Drug-monomer conjugates comprising a prostaglandin or substituted prostaglandin linked via an anhydride linking group at the 1 position may also be prepared by any number of methods known in the art. For example, when R¹ is a free carboxylic acid in prostaglandins and substituted prostaglandins described herein, the reaction of the free carboxylic acid group with another carboxylic acid (e.g. glyceric acid or dihydroxy isobutyric acid) can generate an anhydride linking group at the 1 position. Some examples are shown below:

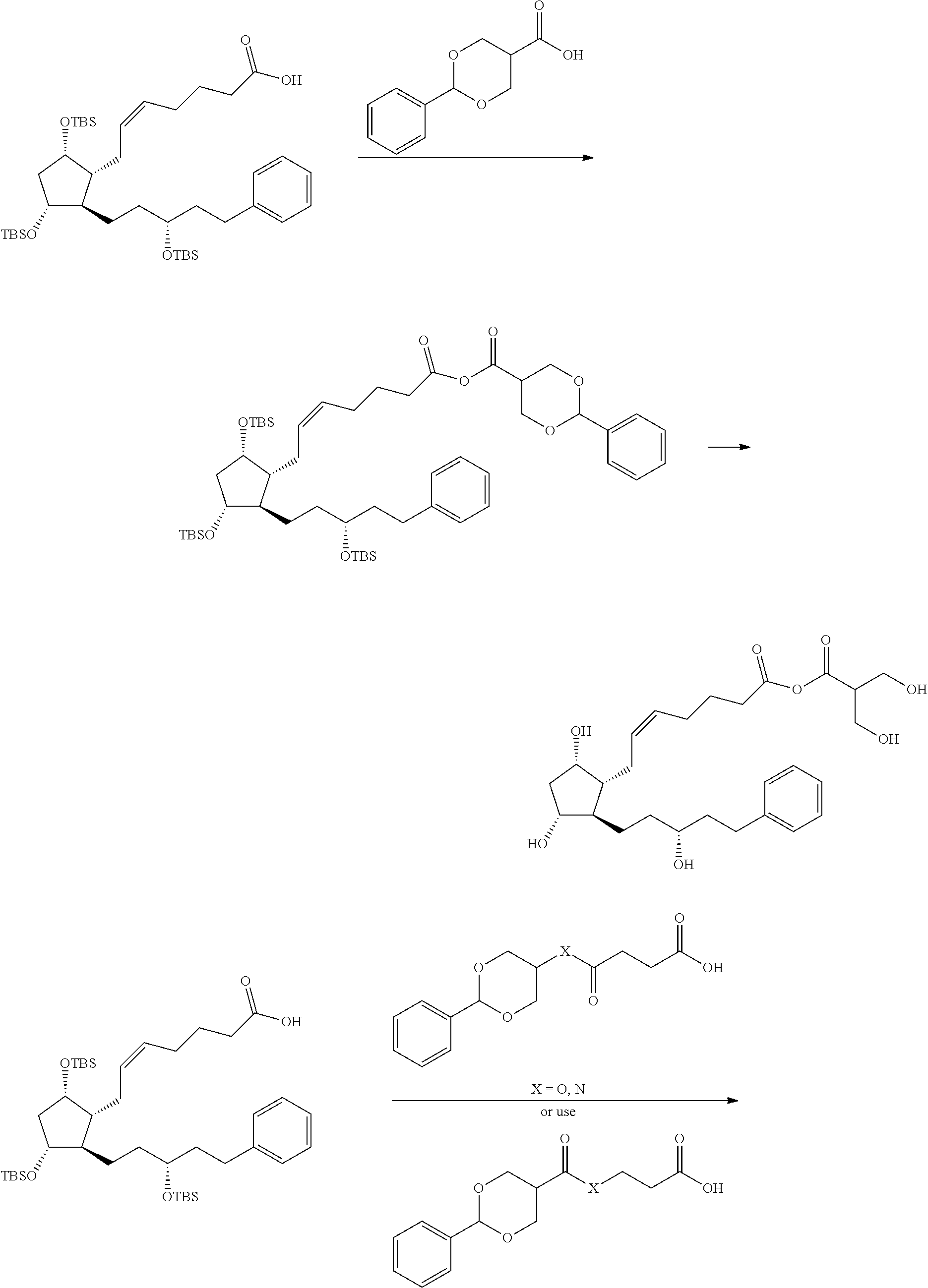

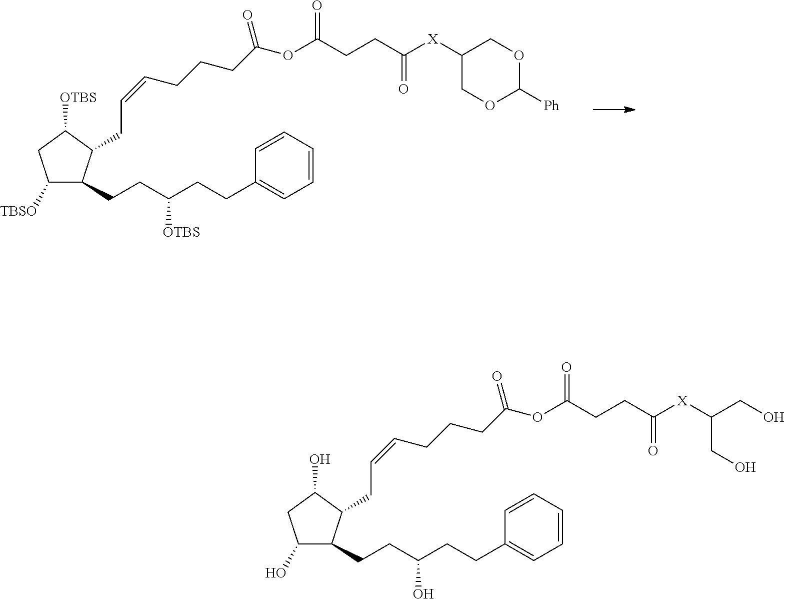

Drug-monomer conjugates comprising a prostaglandin or substituted prostaglandin linked via an ester linking group at the one of the 9, 11 and 15 positions of the drug may also be prepared by esterification methods known in the art, optionally in the presence of a coupling agent. Due to the hydroxy groups at the 9, 11 and 15 positions possessing similar chemical functionality, it may be desirable in some instances to protect one or two of the three hydroxy groups with a suitable protecting group, in order to allow the remaining hydroxy group to be selectively esterified. A list of suitable protecting groups in organic synthesis can be found in T. W. Greene's Protective Groups in Organic Synthesis, 3^{rd} Edition, John Wiley & Sons, 1991. An example of this approach is shown below, where the hydroxy groups at the 9 and 11 positions are protected to allow selective esterification at the 15 position.

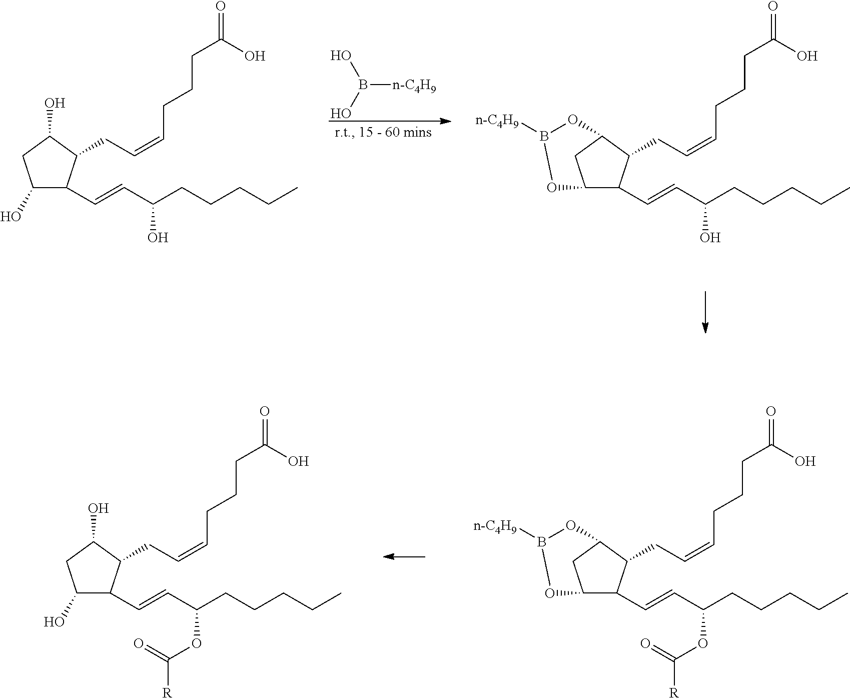

Drug-monomer conjugates comprising a prostaglandin or substituted prostaglandin linked via a carbonate linking group at the one of the 9, 11 and 15 positions of the drug can be made by methods known to those skilled in the art by reaction of, for example, a suitably protected prostaglandin or substituted prostaglandin with a suitable chloroformate. An example is shown below:

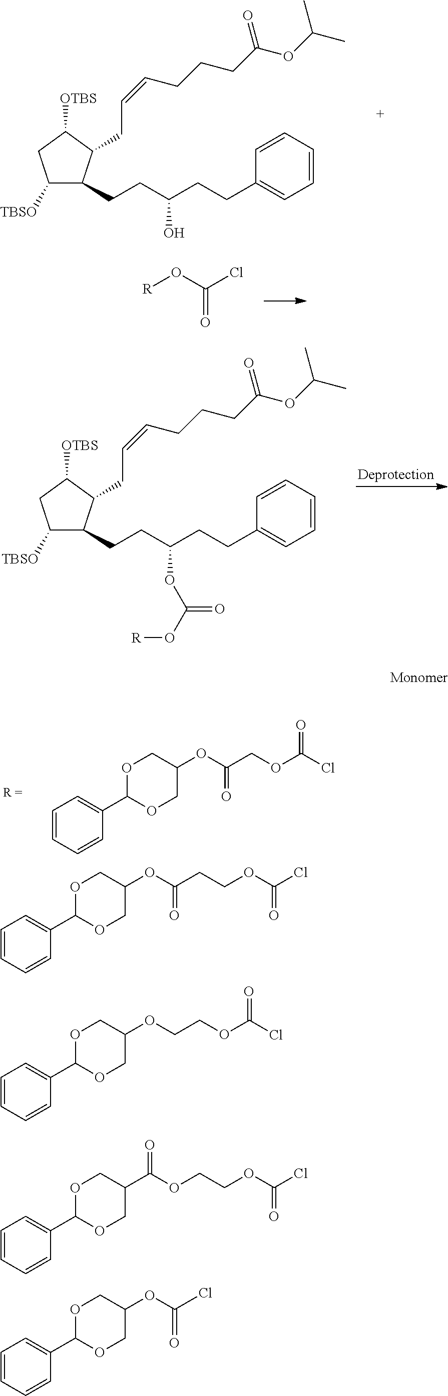

Some review articles outlining general methods for the synthesis of substituted prostaglandins that may be suitable for use in the production of drug-monomer conjugates include the following: Collins, P. W. and Djuric, S. W; Chem. Rev. 1993, 93, 1533-1564 Synthesis of therapeutically useful prostaglandin and prostacyclin analogs, Bindra, J. S.; Bindra, R. Prostaglandin Synthesis, Academic Press: New York, 1977, Mltra, A. The Synthesis of Prostaglandins, Wiley Interscience: New York 1977, Roberts, S. M.; Scheinmann F; New Synthetic Routes to Prostaglandins and Thromboxanes, Academic Press; San Diego 1982, Caton, M. P. L. Tetrahedron, 1979, 35, 2705, Nicolau, K. C.; Gasic, G. P.; Barrette, W. E.; Angew. Chem. Int. Ed. Engl. 1978, 17, 293, and Noyori, R. Suzuki, M.; Angew. Chem. Int. Ed. Engl. 1984, 23, 847.

Diol drug-monomer conjugates of formula (Va) with various “R” groups may be prepared by conjugating a prostaglandin or substituted prostaglandin to a polyfunctional precursor molecule comprising at least two hydroxy groups. Examples of some precursor molecules useful for forming drug-monomer conjugates are shown below:

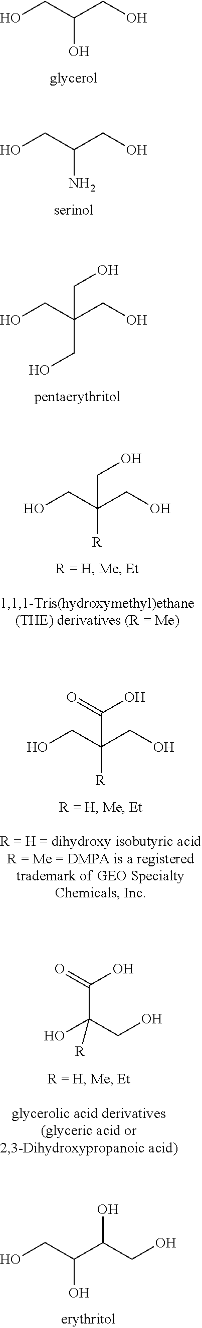

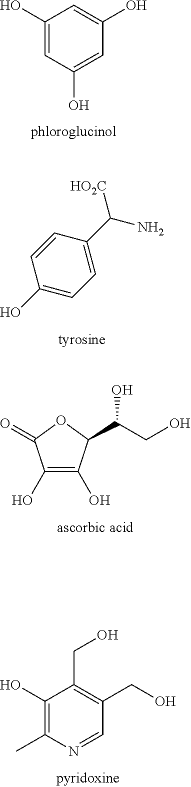

A skilled person would appreciate that the prostaglandin drug moiety (D) may be linked either directly or via the linking group Z, to a hydroxy, amino or carboxylic acid functional group in the precursor molecules in order to form a diol drug-monomer conjugate of formula (Va).

One skilled in the art would also understand that other types of polyfunctional precursor molecules, in addition to the polyhydroxy precursors shown above, may be used to form the drug-monomer conjugates. The choice of precursor molecule may depend on the desired site of attachment on the prostaglandin or substituted prostaglandin (i.e. the 1, 9, 11 or 15 position), the desired linking group (i.e. ester, anhydride or carbonate linking group) linking the drug to the polymer backbone, and the type of bioerodible moiety desired to be present in the polymer backbone. For example, polycarboxylic acid, polyamino, amino acid, hydroxy amino or hydroxy acid precursor molecules (where one or more of the hydroxy groups in the polyhydroxy compounds shown above are replaced with an amino group or carboxylic acid group) can be used to prepare drug-monomer conjugates of the invention. As an example, some polycarboxylic acid precursor molecules are as follows:

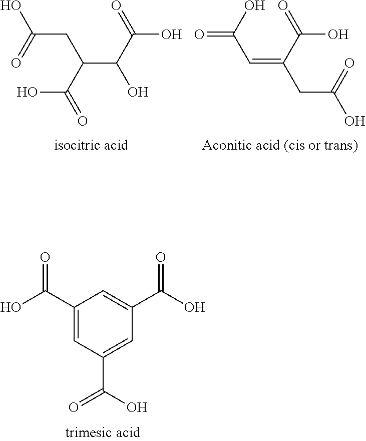

Other polyfunctional precursor molecules that may be used to prepare drug-monomer conjugates of the invention include serine and dihydroxy isobutyric acid.

Polycarboxylic acid, polyamino, amino acid, hydroxy amino or hydroxy acid precursor molecules can be used to prepare dicarboxylic acid drug-monomer conjugates, diamino drug-monomer conjugates, amino acid drug-monomer conjugates, amino alcohol drug-monomer conjugates, or hydroxy acid drug-monomer conjugates, which drug-monomer conjugates are able to react with a suitable monomer comprising compatible chemical functionality to form polymer-drug conjugates of the invention.

The invention also provides a process for making a polymer-drug conjugate as previously defined.

Drug-monomer conjugates described herein polymerise with at least one monomer comprising compatible chemical functionality to form polymer-drug conjugates of the invention.

In some embodiments, monomers that are polymerised with the drug-monomer conjugate of formula (V) to form the bioerodible polymer-drug conjugates of the invention will not only comprise compatible chemical functionality to react with the drug-monomer conjugate but that reaction will also give rise to a bioerodible moiety.

The expression “at least one monomer comprising compatible chemical functionality” used herein typically refers to monomers comprising one or more chemical functional groups that are compatible with, and capable of undergoing reaction with a drug-monomer conjugate of formula (V) during the polymerisation process.

Drug-monomer conjugates of formula (V) may homopolymerise, or they may copolymerise with one or more co-monomers. Thus, the expression “at least one monomer comprising compatible chemical functionality” refers to polymerisation of a drug-monomer conjugate with a monomer of the same type, or with one or more different types of co-monomers, provided that the monomer possesses compatible chemical functionality.

Homopolymerisation can occur when a drug-monomer conjugate of formula (V) contains at least two different terminal reactive functional groups. For example, when Y¹ in formula (V) is a hydroxy group and Y² is a carboxylic acid functional group. Polymerisation of the hydroxy acid drug-monomer conjugate via condensation of the hydroxy and carboxylic acid functional groups therefore forms a polymer-drug conjugate comprising a polymer backbone with ester linkages. A polymer-drug conjugate comprising a polymer backbone with urethane linkages may be similarly formed by homopolymerisation of a drug-monomer conjugate comprising a hydroxy functional group and an isocyanate functional group.

Homopolymerisation with a ring-opening drug-monomer of formula (Vb) can also occur after suitable initiation of the polymerisation reaction.

Copolymerisation can occur when a drug-monomer conjugate of formula (V) contains two terminal reactive functional groups that are of the same type, for example, where Y¹ and Y² in formula (V) are each hydroxy. Such drug-monomer conjugates polymerise with at least one co-monomer comprising compatible chemical functional groups capable of reacting with Y¹ and Y² in order to form a polymer-drug conjugate comprising a polymer backbone that is a copolymer.

Copolymerisation can further occur when a drug-monomer of formula (Vb) undergoes ring-opening polymerisation in the presence of a suitable co-monomer to form polymer-drug conjugate comprising a polymer backbone that is a copolymer. In this instance, the co-monomer may or may not be a ring-opening monomer. Ring-opening co-monomers are generally cyclic co-monomers. The ring-opening co-monomer may comprise at least one cyclic compound selected from the group consisting of lactide, glycolide and ε-caprolactone.

In some embodiments, Y¹ and Y² in a drug-monomer conjugate of formula (V) represent terminal hydroxy groups, such as shown in formula (Va). Those skilled in the art will appreciate that hydroxy groups react with a variety of functional groups such as: isocyanate functionality to form carbamate or urethane linkages; carboxylic acid functionality to produce ester linkages; carboxylic acid halide functionality to produce ester linkages; ester functionality to produce trans-esterified ester linkages; and anhydride functionality (including cyclic anhydride groups) to produce ester linkages. The expression “compatible chemical functionality” can therefore refer to functionality or groups such as isocyanate, carboxylic acid, carboxylic acid halide, ester, amine and anhydride (including cyclic anhydride groups) groups.

Accordingly, the expression “at least one monomer comprising compatible chemical functionality” used herein typically refers to monomers comprising one or more compatible chemical functional groups selected from isocyanate, carboxylic acid, carboxylic acid halide, ester (including cyclic ester or lactone groups), anhydride (including cyclic anhydride groups), carbonate (including cyclic carbonate groups), amide (including cyclic amide or lactide groups) and amino groups, and combinations thereof. Examples of such monomers can be selected from the group consisting of a polyisocyanate, a polyol, a polyacid, a polyacid halide, a polyester, a polyanhydride, a polycarbonate, a polyamide, a polyamine, and combinations thereof. In embodiments of the invention the monomer comprising compatible functionality is selected from the group consisting of a diisocyanate, a diacid, a diacid halide, a diester (in particular, a divinyl ester), and a dianhydride.

In some embodiments, the present invention provides a method of preparing a polymer-drug conjugate according to any one of the embodiments described herein, the method comprising polymerising a drug-monomer of formula:

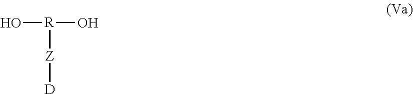

with monomer selected from the group consisting of: polyacid halides, polycarboxylic acids, polycarboxylic acid esters, polycarboxylic anhydrides, polyisocyanates, polyamines, cyclic esters and cyclic carbonates.

In some embodiments, the drug-monomer conjugate of formula (V) is polymerised with at least one monomer selected from the group consisting of: diacid halides, dicarboxylic acids, dicarboxylic acid esters in particular divinyl esters, dicarboxylic anhydrides, diisocyanates in particular hexamethylene diisocyanate (HDI), amino acid based diisocyanates (such as esters of lysine diisocyanate (for example ethyl ester of lysine diisocyanate (ELDI)) and divaline diisocyanate 1,3-propane diol (DVDIP)), lactones and cyclic carbonates.

Those skilled in the art will also recognise that polymerisation of a diol of formula (Va) with a polyisocyanate, polyacid or polyester may also take place in the presence of one or more other types of polyols, lactones or lactides (e.g. polyester polyols). The structures of these one or more other types of polyols may or may not comprise one or more drug moieties. An example of this second type of polyol is caprolactone. The polymer-drug conjugates so-formed may or may not have a drug loading of less than 50 mol %. For example where diol of formula (V) is polymerised in the presence of an equimolar amount of caprolactone and 2 molar equivalents of diisocyanate, the polyurethane so-formed will typically comprise the residues of the three components in the ratio of 1:1:2. Such conjugates are contemplated by the present invention. Such polymer systems may provide a useful means of modifying the physical properties of the polymer conjugates.

Suitable polyisocyanates that may be used to prepare the polymer-drug conjugates include aliphatic, aromatic and cycloaliphatic polyisocyanates and combinations thereof. Specific polyisocyanates include, but are not limited to, diisocyanates such as hexamethylenediisocyanate and alkyl esters of lysine diisocyanate (for example C1-3 alkyl esters of lysine diisocyanate, in particular, ethyl ester of lysine diisocyanate—ELDI); and combinations thereof.

In some embodiments, in preparing polymer-drug conjugates of the invention, the polymerisation of a drug-monomer conjugate of formulae described herein and a monomer comprising compatible chemical functionality can optionally occur in the presence of one or more co-monomers.

In some embodiments, co-monomer may be a monomer comprising at least one active-hydrogen group. The polymerisation of a drug-monomer conjugate as described herein with a monomer comprising compatible functionality and a monomer comprising at least one active-hydrogen group results in the incorporation of a hydrophilic group in the polymer backbone of the polymer-drug conjugate.

In some embodiments, the active-hydrogen group containing monomer is a macromonomer comprising a plurality of active-hydrogen groups. The active-hydrogen groups may be selected from hydroxy, amine and carboxylic acid groups, and combinations thereof.

Active-hydrogen groups, as well as monomers comprising active-hydrogen groups are described herein. Such monomers will generally contain at least one functional group capable of reacting with at least one selected from the group consisting of the monomer-drug conjugate of formula (V) and the monomer comprising compatible chemical functionality.

That is, the active-hydrogen group containing monomer is capable of reacting with the monomer-drug conjugate of formula (V) and/or the monomer comprising compatible chemical functionality. The active-hydrogen group containing monomer may contain at least two reactive functional groups.

In some embodiments, the active-hydrogen group containing monomer comprises at least one reactive functional group selected from the group consisting of hydroxy, isocyanate, carboxylic acid, carboxylic acid halide, ester, anhydride (including cyclic anhydride groups), amide, and amino groups, and combinations thereof, capable of reacting with a drug-monomer conjugate of formula (V), or at least one monomer comprising compatible chemical functionality.

An active-hydrogen containing monomer (for example, a macromonomer) is generally pre-formed, then added to the mixture of monomers used to prepare the polymer-drug conjugate.

In some embodiments, an active-hydrogen group containing monomer may be added to a monomer mixture comprising a drug-monomer conjugate of formula (V) (such as a diol where Y¹ and Y² are each hydroxy) and at least one monomer (such as a polyisocyanate, polyacid or polyester polyol) comprising compatible chemical functionality. In such instances, it is preferable that the active-hydrogen group containing monomer comprises at least two functional groups that are capable of reacting with the functional groups of the monomer comprising compatible chemical functionality to thereby incorporate the active-hydrogen group containing monomer into the polymer-drug conjugate as a hydrophilic group in the polymer backbone

In some embodiments the polymer-drug conjugates of the invention may be formed by polymerising a diol drug-monomer conjugate of formula (V) with an active-hydrogen group containing monomer comprising a polymeric or oligomeric unit, and at least two terminal groups comprising compatible chemical functionality. In such instances, the terminal groups of the active-hydrogen group containing monomer are capable of reacting with the hydroxy groups in the monomer of formula (V), resulting in the incorporation of a hydrophilic group into the polymer backbone of the polymer-drug conjugate.

In some embodiments of a polymer-drug conjugate of the invention, the polymer backbone comprises a copolymer selected from the group consisting of poly(urethane-ethers), poly(ester-ethers), poly(urethane-esters), and poly(ester-urethanes). The ether or ester component of the copolymer may provide a hydrophilic segment in the polymer backbone

In some embodiments the ether component may be introduced to the polymer backbone by polymerising a polyether polyol as an active-hydrogen group containing monomer (for example, a PEG macromonomer), with a drug-monomer conjugate of the invention and at least one monomer comprising compatible chemical functionality.

In some embodiments the ester component may be introduced to the polymer backbone by polymerising a polyester polyol as an active-hydrogen group containing monomer, with a drug-monomer conjugate of the invention and at least one monomer comprising compatible chemical functionality.

In some embodiments, an active-hydrogen group containing monomer may be polymerised in situ during synthesis of the polymer-drug conjugate of the invention, resulting in the subsequent incorporation of a hydrophilic polymeric or oligomeric group in the polymer backbone of the conjugate.

In some embodiments the polymer-drug conjugates of the invention may be formed by polymerising a monomer mixture comprising a diol of formula (Va), at least one monomer comprising compatible chemical functionality, and at least active-hydrogen group containing monomer. The active-hydrogen group containing monomer will generally comprise reactive functional groups that are capable of reacting with the diol of formula (Vc) and/or the monomer comprising compatible chemical functionality. In this manner, the active-hydrogen group containing monomer can be incorporated as a hydrophilic group in the polymer backbone of the polymer-drug conjugate.

The present invention also provides a method for preparing a polymer-drug conjugate comprising as part of its polymer backbone a moiety of general formula (Ic):

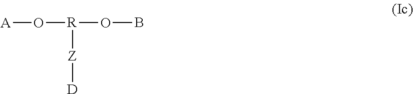

where:
A and B, which may be the same or different, represent the remainder of the polymer backbone and are (i) attached to the —O—R(ZD)-O— moiety as shown in formula (Ic) via a bioerodible moiety, and (ii) each formed from monomeric units that are coupled via bioerodible moieties;
R is an optionally substituted hydrocarbon;
Z is a linking group;
D is a prostaglandin drug of formula (XX); and
D and Z together form an ester, anhydride or carbonate linking group.
   said process comprising a step of polymerising a drug-monomer conjugate of formula (Va):

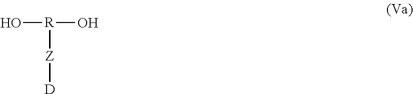

where:
R, Z and D are as defined above;
   with at least one monomer comprising compatible chemical functionality.

The reaction of the diol drug-monomer conjugate of formula (Va) with at least one monomer comprising compatible chemical functionality may optionally take place in the presence of a monomer comprising at least one active-hydrogen group. Examples of suitable active-hydrogen group containing monomers are described above.

In one embodiment, a polymer-drug conjugate of the invention is obtained by polymerising a drug-monomer conjugate of formulae (V), (Va) or (Vb) in the presence of at least one monomer comprising compatible chemical functionality selected from the group consisting of a polyisocyanate, a polyol, a polyacid, a polyester, a poly(ester-ether), a polyanhydride, a polyamine, and combinations thereof.

In one embodiment, a polymer-drug conjugate of the invention is obtained by polymerising a drug-monomer conjugate of formulae ((V), (Va) or (Vb) in the presence of a polyisocyanate and at least one selected from the group consisting of a polyacid, a polyester, a polyester polyol, a poly(ester-ether), a polyester hydroxy acid and a polyether polyol.

In one embodiment, a polymer-drug conjugate of the invention is obtained by polymerising a drug-monomer conjugate of formulae (V), (Va) or (Vb) in the presence of a polyisocyanate and at least one selected from the group consisting of a polyester polyol, a poly(ester-ether), a polyester hydroxy acid, and a polyether polyol.

Suitable polyisocyanates that may be used to prepare the polymer-drug conjugates include aliphatic, aromatic and cycloaliphatic polyisocyanates and combinations thereof. Specific polyisocyanates may be selected from the group consisting of m-phenylene diisocyanate, p-phenylene diisocyanate, 2,4-toluene diisocyanate, 2,6-toluene diisocyanate, 1,6-hexamethylene diisocyanate, 1,4-hexamethylene diisocyanate, 1,3-cyclohexane diisocyanate, 1,4-cyclohexane diisocyanate, hexahydro-toluene diisocyanate and its isomers, isophorone diisocyanate, dicyclo-hexylmethane diisocyanates, 1,5-napthylene diisocyanate, 4,4′-diphenylmethane diisocyanate, 2,4′ diphenylmethane diisocyanate, 4,4′-biphenylene diisocyanate, 3,3′-dimethoxy-4,4′-biphenylene diisocyanate, 3,3′-dimethyl-diphenylpropane-4,4′-diisocyanate, 2,4,6-toluene triisocyanate, 4,4′-dimethyl-diphenylmethane-2,2′,5,5′-tetraisocyanate, polymethylene polyphenhyl polyisocyanates, divaline diisocyanate 1,3-propane diol, and alkyl esters of lysine diisocyanate (preferably ethyl ester of lysine diisocyanate) and combinations thereof. Preferred polyisocyanates include 1,6-hexamethylene diisocyanate (HDI), alkyl esters of lysine diisocyanate (preferably C₁₋₃ alkyl esters of lysine diisocyanate, in particular, ethyl ester of lysine diisocyanate), and divaline diisocyanate 1,3-propane diol (DVDIP).

Suitable polyacids may be selected from the group consisting of oxalic acid, fumaric acid, maleic acid, succinic acid, glutaric acid, adipic acid, pimelic acid, suberic acid, azelaic acid, sebacic acid, phthalic acid, dodecanediacid, isophthalic acid, terephthalic acid, dodecylsuccinic acid, napthalene-2,6-dicarboxylic acid, naphthalene-2,7-dicarboxylic acid, cyclohexane dicarboxylic acid, itaconic acid, malonic acid, mesaconic acid, and combinations thereof. Preferred polyacids include maleic acid and succinic acid.

Suitable polyester polyols may be selected from the group consisting of polycaprolactone diol (PCLD), poly(DL lactide) (DLLA) and poly(lactic acid-co-glycolic acid) (PLGA), and combinations thereof.

Suitable polyether polyols may be selected from the group consisting of poly(ethylene glycol) (PEG), poly(propylene glycol), and combinations thereof.

A suitable poly(ester-ether) may be poly(1,5-dioxepan-2-one) (PDOO). Suitable hydroxy acids include lactic acid and glycolic acid, and combinations thereof.

Techniques, equipment and reagents well known in the art can advantageously be used to prepare the polymer-drug conjugates in accordance with the invention.

For example, polyurethanes might be prepared batch wise by mixing all components together and waiting until an exotherm occurs followed by casting the mixture into a container. The mixture can be subsequently heated to drive the reaction. When adopting this approach, the components to be mixed might first be made up into two parts before mixing: Part-1 might include a drug-monomer conjugate in accordance with the invention and one or more of: a polyol (e.g. polyester polyol), a chain extender, blowing agent (e.g. water), catalyst, and surfactants etc. Part-2 will generally comprise the polyisocyanate. Part-1 or Part-2 can also contain other additives such as fillers, etc.

The polyurethanes might also be prepared as a prepolymer that is subsequently reacted with a chain extender. For example, through suitable adjustment of molar ratios, an isocyanate terminated pre-polymer may be prepared by mixing Parts-1 and -2 mentioned above. The isocyanate terminated polymer could then be reacted with a chain extender/branching molecule such as a short chain diol (e.g. 1,4-butanediol) or polyol (such as a triol). Alternatively, through suitable adjustment of molar ratios, the prepolymer could be produced such that it was hydroxy terminated. This hydroxy terminated prepolymer could then be reacted with a polyisocyanate to produce the desired polyurethane.

Variables such as the choice of co-monomers and the means to produce the polymers can also assist with the production of highly amorphous and/or flexible polymers. For example, using monomers such as caprolactone or polyester polyols such as polycaprolactone diol can decrease the crystallinity and increase the flexibility of the resulting polymer. In addition, polyesters such as PLGA, PDOO and polyethers such as poly(ethylene glycol) may increase the hydrophilicity of the polymer-drug conjugates.

The polyurethane forming reactions can be carried out in a range of different equipment including batch kettles, static mixers, reactive injection moulders or extruders. It also may be advantageous to heat the reagents prior to or during the reaction process to improve their solubility or to enhance their reactivity. The reaction process may also be conducted in solvent.

Suitable polyacids that may be used to prepare the polymer-drug conjugates include aliphatic, aromatic and cycloaliphatic polyacids and combinations thereof. Specific polyacids include, but are not limited to the following, succinic acid, adipic acid, sebacic acid, and malonic acid. Esters, diesters and anhydrides of the above diacids are also suitable in the process of the invention.

Polyesters might be prepared batch wise by mixing all components together with heating and continued stirring. A condensate of the reaction such as water or low molecular weight alcohol (depending if acids or esters are used as the co-monomer) can be removed by distillation. To promote further reaction produce higher molecular weight polyester the temperature may be increased and vacuum applied.

A polycondensation catalyst well known to those skilled in the art can be included in the reaction mixture to increase the rate of polymerisation.

The reaction may also be conducted in an appropriate solvent to help increase the rate of polymerisation. The solvent will generally be selected to have only minimal solubility with the condensate (e.g. water or low molecular weight alcohol). For example the reaction may be carried out in toluene and a toluene/condensate mixture distilled off continuously and the condensate allowed to separate in a Dean—Stark trap.

Where the polyesters are prepared using a carboxylic acid halide monomer, those skilled in the art will appreciate that the condensation reaction is driven by the removal of HX (where X is a halide). For example, if a di-acid chloride co-monomer is with the monomer-drug conjugate of formula (V), HCl will be liberated from the reaction. Such a reaction may be carried out in solution at an elevated temperature to drive the reaction. It is also possible to add an appropriate base to form a salt with the liberated acid halide. For example an excess of triethyl amine may be included in a reaction mixture containing a 1:1 molar ratio of a di-acid chloride co-monomer and the drug-monomer conjugate of formula (V). The reaction will afford the desired polymer-drug conjugate and a triethyl-amine hydrochloride salt.

With all such polycondensation reactions, it is possible to some extent to control the molecular weight of the resulting polyester, its degree of branching (through control of monomer functionality) and its end group functionality by adjustment of the molar ratio's and the functionality of the monomers used in the reaction.

Careful selection of co-monomers/reaction conditions etc may also be required for a given drug-monomer conjugate in order to produce a polymer conjugate with appropriate drug loading as well as have mechanical properties, bioactive release rate, formability etc.

When polymer-drug conjugates of the invention are fully bioerodible, all repeat units that make up the polymer backbone will be coupled via a bioerodible moiety. Accordingly, any monomer or macromonomer used in the preparation of the conjugates shall not contain repeat units that are coupled by a non-bioerodible moiety such as an ether.

The polymer backbone of the polymer-drug conjugates of the present invention may have a molecular weight of about 250 Daltons to about 2 mM Daltons, preferably from 500 Daltons to 500,000 Daltons, more preferably from 2,000 Daltons to 200,000 Daltons.

The polymer-drug conjugates of the present invention can accommodate high drug loadings, minimising the amount of material required to deliver a dose of the drug. A drug loading selected from the group consisting of at least 10% by weight, at least 20% by weight, and at least 30% by weight relative to the total weight of the polymer may be achieved.

The drug loading may also be expressed in terms of its mol % relative to the total number of moles of monomer that forms the polymer. Generally, the polymer-drug conjugate will comprise at least 10, at least 25, at least 35, at least 45 or up to 50 mol % of the drug, relative to the total number of moles of monomer that form the polymer.

In some embodiments, the polymer-drug conjugate will comprise up to 10, up to 20, up to 30, up to 40 and even up to 50 mol % of conjugated drug, relative to the total number of moles of monomer that form the polymer.

As described above, prostaglandin drug conjugates to the backbone of polymer-drug conjugates of the invention are releasable. Upon being released, the drug is bioactive or will be converted in vivo or in vitro to a bioactive form (e.g. as in the case of a prodrug).

As the drug moiety (D) is linked to the polymer backbone via an ester, anhydride or carbonate linkage, cleavage of the drug from the polymer-drug conjugate will generally proceed via a hydrolysis reaction. Hydrolysis of the ester, anhydride or carbonate linkage under appropriate conditions allows the drug to be released from the conjugate. One skilled in the art would be able to determine appropriate conditions under which an ester, anhydride or carbonate will hydrolyse to release the drug. A test to evaluate drug release is described herein in the Examples. When the polymer-drug conjugate is bioerodible, the hydrolysis of the linking group preferably proceeds at a faster rate than the rate of erosion of the polymer backbone.

Hydrolysis of the ester, anhydride or carbonate linkage may be influenced by the pH of the surrounding environment. For example, a more alkaline environment (pH 8.0 or higher) may help to promote hydrolysis and hence drug release.

It has been found that the polymer-drug conjugates according to the invention are particularly useful in applications where controlled delivery of the drug is required. Accordingly, the polymer-drug conjugate of the invention can provide for a controlled release drug delivery system. By “controlled” release is meant that release of a dose of the drug is controlled in a manner that enables the drug to be released over a desired period of time. Controlled release may be zero order release, first order release, or delayed release of the drug.

In some embodiments, the drug may be released from the polymer-drug conjugate such that it provides for a sustained release drug delivery system. By “sustained” release is meant that a dose of the drug is released over a prolonged period of time, for example, over several days to weeks. This can enable a therapeutic effect to be maintained during a course of treatment over a desired period of time. This can be advantageous as it avoids the need for repeated administrations of the conjugate during the treatment.

In some embodiments, the controlled release of the prostaglandins and substituted prostaglandins occurs over a period selected from the group consisting of at least 15 days, at least 30 days, at least 45 days, at least 60 days, and at least 90 days. Controlled release over an extended period of time may be advantageous in the case of an implant to allow for easier co-ordination with a patient's visitation with a medical practitioner.

In some embodiments, a polymer-drug conjugate of the invention is capable of releasing the drug at a level of at least about 20 ng/24 hours. In embodiments of the invention, the drug is released at a level of at least about 50 ng/24 hours. Such release levels are typically at or above therapeutic levels for prostaglandins and substituted prostaglandins.

In another aspect, the present invention also provides a drug delivery system comprising a polymer-drug conjugate as described herein. The drug delivery system can facilitate administration of a prostaglandin or substituted prostaglandin to a subject.

To encourage drug release the drug delivery system of the invention will, in some embodiments, comprise a hydrophilic component.

The hydrophilic component may be mixed or blended with a polymer-drug conjugate of the invention, or it may be incorporated in the polymer-drug conjugate as a component of the polymer backbone. The inclusion of a hydrophilic component can aid drug release.

In some embodiments, the hydrophilic component may be provided by at least one selected from the group consisting of (i) the polymer backbone of the polymer-drug conjugate comprising at least one hydrophilic group, and (ii) at least one hydrophilic polymer in admixture with the polymer-drug conjugate. The drug delivery system may also comprise a combination of (i) and (ii).

Polymer-drug conjugates comprising a polymer backbone comprising a hydrophilic group are described herein. As discussed above, the hydrophilic group may be provided by (i) at least one hydrophilic group incorporated in the conjugate as part of the polymer backbone, (ii) at least one hydrophilic group being covalently attached to and pendant from the polymer backbone, or (iii) combinations thereof. The hydrophilic group may be provided by or derived from a monomer comprising at least one active-hydrogen containing group, and may comprise a oligomeric or polymeric moiety comprising a plurality of active-hydrogen groups. Active-hydrogen groups are described herein. Such polymer-drug conjugates may be incorporated in a drug delivery system of the invention.

In some embodiments, polymer-drug conjugates comprising a hydrophilic group as a part of the polymer backbone comprise at least one oligomeric or polymeric moiety selected from the group consisting of poly(ethylene glycol), poly(lactic acid-co-glycolic acid) (PLGA), poly(1,5-dioxepan-2-one) (PDOO), poly(glycerol acetate) (PGAc), poly(hydroxy butyrate), poly(glycerol phosphate), an amino acid polymer (such as polylysine, polyglutamic acid, etc), or an amino acid oligomer, or combination of, or a copolymer of, such polymeric or oligomeric moieties.

In some embodiments, a drug delivery system of the invention comprises at least one hydrophilic polymer in admixture with the polymer-drug conjugate. In such embodiments, the polymer-drug conjugate may or may not comprise a hydrophilic group as described herein. In one form, the polymer-drug conjugate is blended with the hydrophilic polymer.

In some embodiments of a drug delivery system of the invention, the hydrophilic polymer is derived from at least one monomer comprising at least one active-hydrogen group.

Examples of such monomers include low molecular weight diols (preferably C2-C4 diols such as ethylene glycol, propane diol, propylene glycol, butane diol etc), amino acids, lactic acid, glycolic acid, hydroxy acids (preferably hydroxybutyric acid, etc), 1,5-dioxepan-2-one, glycerol acetate and glycerol phosphate. The hydrophilic polymer may comprise a single type of monomeric unit. The hydrophilic polymer may be a copolymer comprising a combination of two or more different types monomeric units derived from such monomers.

In some embodiments, the hydrophilic polymer is at least one selected from the group consisting of poly(ethylene glycol), poly(lactic acid-co-glycolic acid) (PLGA), poly(1,5-dioxepan-2-one) (PDOO), poly(glycerol acetate) (PGAc), poly(hydroxy butyrate), poly(glycerol phosphate), an amino acid polymer, and combinations thereof. In one form of a drug delivery system of the invention, the hydrophilic polymer is poly(ethylene glycol).

The drug delivery system may comprise a single type of hydrophilic polymer, or it may comprise a combination of two or more different types of hydrophilic polymer in admixture with the polymer-drug conjugate.

A hydrophilic polymer in admixture with the polymer-drug conjugate may be of any suitable molecular weight. In some embodiments, the hydrophilic polymer has a molecular weight in the range of from about 200 to about 15,000, preferably in the range of from about 500 to about 5,000.

In a preferred embodiment, the drug delivery system comprising a polymer-drug conjugate of the invention in admixture with poly(ethylene glycol). The poly(ethylene glycol) preferably has a molecular weight in the range of from of from about 1000 to about 3,000.

The use of a hydrophilic component in combination with a polymer-drug conjugate comprising an ester, anhydride or carbonate linked prostaglandin drug may help to promote drug release from the polymer conjugate. Without wishing to be limited by theory, it is believed that a hydrophilic component in the vicinity of the pendant drug moiety can help to facilitate drug release by attracting water molecules to vicinity of the linking group conjugating the drug to the polymer backbone, thereby triggering hydrolysis of the linking group and resulting in drug release.

In some embodiments, polymer-drug conjugates of the invention may provide for substantially zero-order release of the drug. Zero order release can help ensure that a steady amount of drug is released over time. In some embodiments, the polymer-drug conjugate of the invention provides for zero-order release of a therapeutically effective amount of the drug over a period of time of at least 7 days. In some embodiments, zero-order release of a therapeutically effective amount of the drug may occur over a period selected from the group consisting of at least 15 days, at least 30 days, at least 45 days, at least 60 days, and at least 90 days. A zero order release profile may be achieved even when the polymer-drug conjugate is fully dissolved in a solvent.

Advantageously, polymer-drug conjugates of the invention do not suffer from a “burst effect”, where a higher than optimal dose of drug is initially released. The burst effect can be undesirable, as overdosing on the drug can result.

Polymer-drug conjugates of the invention may be formulated in a pharmaceutical composition. In this regard, the polymer-drug conjugate or drug delivery system may be blended with a pharmacologically acceptable carrier. By “pharmacologically acceptable” is meant that the carrier is suitable for administration to a subject in its own right. In other words, administration of the carrier to a subject will not result in unacceptable toxicity, including allergenic responses and disease states. The term “carrier” refers to the vehicle with which the conjugate is contained prior to being administered.

In some embodiments, the carrier is a pharmaceutically acceptable solvent. A suitable pharmaceutically acceptable solvent may be an aqueous solvent, such as water. The polymer-drug conjugate of the invention and the drug delivery system of the invention may advantageously be soluble in the solvent.

Polymer-drug conjugates of the invention may be prepared in suitable forms for administration to a subject.

The form of the polymer-drug conjugate or the drug delivery system may be adjusted to be suited to the required application such as a coating, film, pellet, fibres, laminate, foam etc. The delivery system may in its simplest form be the conjugate provided in a desired shape, for example a rod or more intricate shape. To promote surface area contact of the conjugate with a biological environment, the conjugate may also be provided in the form of a coating on substrate, or as an article have porosity (e.g. an open cell foam).

Different physical structures can have different masses, which can result in different rates of drug release from essentially the same polymer composition.

The adjustment of the form of the polymer to suit the application and further to adjust the form to further control the drug release profile can provide an additional advantage over purely compositional and polymer structural means to control the release profile of the drug.

Polymer-drug conjugates in accordance with the invention or materials containing a polymer-drug conjugate or a drug delivery system in accordance with the invention can be formed into an article or device. The article or device may be fabricated in a range of forms. Suitably, the article or device is a medical device. The polymer-drug conjugates in accordance with the invention can also be incorporated or made into coatings for target in vitro and in vivo applications.

The drug polymer-conjugates in accordance with the invention or materials containing the polymer-drug conjugate in accordance with the invention can be formed into an article or device suitably shaped to facilitate delivery to the eye. One such device is a rod-shaped implant able to be housed within the lumen of a 20 to 23 gauge needle. The outer diameter of the implant would be less than 0.5 mm, preferably about 0.4 mm and more preferably 0.3 mm. The length of the implant can be selected to deliver the required dose of drug,

The resultant implant could be a solid, a semi-solid or even a gel. A solid implant would comprise material with a glass transition temperature (as measured by differential scanning calorimetry) above 37° C., a semi-solid would have a glass transition temperature at or just below 25-37° C. A gel could be formed by appropriate formulation of the drug-polymer conjugate with an appropriate plasticiser.

The rod-shaped implant can be of a number of different structural forms. Firstly the rod-shaped implant can consist solely of the polymer-drug conjugate or as a blend with another appropriate bioerodible polymer (for example PGLA or a degradable polyurethane).

Another possibility is to make the rod-shaped implant as a bi-component structure where the polymer-drug conjugate can either be incorporated in the out or inner layers. Incorporating the polymer-drug conjugate in the outer layer could be done to give a measured dose.

Additionally the inner layer bioerodible polymer could be to provide structural integrity to allow the delivery via the needle. Additionally the inner polymer could be designed to degrade either faster or slower than the polymer-drug conjugate layer. This could be to alter the rate of bioerosion or the implant.

It is also possible to produce rod-shaped implants containing the polymer-drug conjugate of different shapes without affecting the rate of drug release from the implant.

Possible means for producing the rod-fibre implants described above include:
Melt extrusion of the polymer-drug conjugate or a material containing the polymer-drug conjugate through a shaped die.
Simultaneous bi-component extrusion of the polymer-drug conjugate and other materials forming the outer or inner layers through an appropriate die.
Sequential overcoating extrusion of one polymer later with another. For example a core polymer fibre of PLGA could be melt overcoated with a polymer containing the drug polymer conjugate.
It is also possible to solution coat an appropriate inner polymer carrier material (e.g. PLGA) with a solution containing the drug polymer conjugate.

The present invention also provides a sustained drug delivery system comprising a polymer-drug conjugate of the invention. In one embodiment, the sustained drug delivery system may be in the form of an implant. The sustained drug delivery system may enable prostaglandins or substituted prostaglandins to be administered over a sustained period of time, such as for example, for at least at least 15 days, for at least 30 days, for at least 45 days, for at least 60 days, or for at least 90 days. A sustained release drug delivery system may be a more convenient way to administer prostaglandins and substituted prostaglandins, as it enables therapeutic levels of the drug to be continuously administered over an extended period time and allows the drug therapy schedule to be matched with a patient's visitation schedule to a medical or health practitioner.

In another aspect, the present invention provides an implant for the treatment of glaucoma in a subject, wherein the implant comprises a polymer-drug conjugate or a drug delivery system of any one of the embodiments described herein.

The implant may be in any form suitable for administration to the eye. In some embodiments, the implant is in the form of a solid article for placement in the eye of the subject.

The polymer-drug conjugates and drug delivery systems of the invention may be useful for delivering prostaglandins and substituted prostaglandins for the treatment of glaucoma.

In another aspect, the present invention provides a method of treatment of glaucoma in a subject suffering glaucoma in one or both eyes, the method comprising administering to an eye afflicted with glaucoma a polymer-drug conjugate or a drug delivery system according to any one of the embodiments described herein.

In some embodiments, the polymer-drug conjugate or drug delivery system may be in the form of a solid polymer article (such as a particle, rod or pellet) and the method comprises implanting the article into the affected eye of the subject. In one form, the method comprises depositing the polymer article in the lumen of a syringe needle and injecting the polymer article into the eye.

In another aspect, the present invention also provides use of a polymer-drug conjugate as described herein in manufacture of a medicament for treatment of glaucoma in at least one eye of a subject.

In another aspect, the present invention also provides use of a drug delivery system as described herein in manufacture of a medicament for treatment of glaucoma in at least one eye of a subject.

In this specification “optionally substituted” is taken to mean that a group may or may not be substituted or fused (so as to form a condensed polycyclic group) with one, two, three or more of organic and inorganic groups (i.e. the optional substituent) including those selected from: alkyl, alkenyl, alkynyl, carbocyclyl, aryl, heterocyclyl, heteroaryl, acyl, aralkyl, alkaryl, alkheterocyclyl, alkheteroaryl, alkcarbocyclyl, halo, haloalkyl, haloalkenyl, haloalkynyl, haloaryl, halocarbocyclyl, haloheterocyclyl, haloheteroaryl, haloacyl, haloaryalkyl, hydroxy, hydroxyalkyl, hydroxyalkenyl, hydroxyalkynyl, hydroxycarbocyclyl, hydroxyaryl, hydroxyheterocyclyl, hydroxyheteroaryl, hydroxyacyl, hydroxyaralkyl, alkoxyalkyl, alkoxyalkenyl, alkoxyalkynyl, alkoxycarbocyclyl, alkoxyaryl, alkoxyheterocyclyl, alkoxyheteroaryl, alkoxyacyl, alkoxyaralkyl, alkoxy, alkenyloxy, alkynyloxy, aryloxy, carbocyclyloxy, aralkyloxy, heteroaryloxy, heterocyclyloxy, acyloxy, haloalkoxy, haloalkenyloxy, haloalkynyloxy, haloaryloxy, halocarbocyclyloxy, haloaralkyloxy, haloheteroaryloxy, haloheterocyclyloxy, haloacyloxy, nitro, nitroalkyl, nitroalkenyl, nitroalkynyl, nitroaryl, nitroheterocyclyl, nitroheteroayl, nitrocarbocyclyl, nitroacyl, nitroaralkyl, amino (NH₂), alkylamino, dialkylamino, alkenylamino, alkynylamino, arylamino, diarylamino, aralkylamino, diaralkylamino, acylamino, diacylamino, heterocyclamino, heteroarylamino, carboxy, carboxyester, amido, alkylsulphonyloxy, arylsulphenyloxy, alkylsulphenyl, arylsulphenyl, thio, alkylthio, alkenylthio, alkynylthio, arylthio, aralkylthio, carbocyclylthio, heterocyclylthio, heteroarylthio, acylthio, sulfoxide, sulfonyl, sulfonamide, aminoalkyl, aminoalkenyl, aminoalkynyl, aminocarbocyclyl, aminoaryl, aminoheterocyclyl, aminoheteroaryl, aminoacyl, aminoaralkyl, thioalkyl, thioalkenyl, thioalkynyl, thiocarbocyclyl, thioaryl, thioheterocyclyl, thioheteroaryl, thioacyl, thioaralkyl, carboxyalkyl, carboxyalkenyl, carboxyalkynyl, carboxycarbocyclyl, carboxyaryl, carboxyheterocyclyl, carboxyheteroaryl, carboxyacyl, carboxyaralkyl, carboxyesteralkyl, carboxyesteralkenyl, carboxyesteralkynyl, carboxyestercarbocyclyl, carboxyesteraryl, carboxyesterheterocyclyl, carboxyesterheteroaryl, carboxyesteracyl, carboxyesteraralkyl, amidoalkyl, amidoalkenyl, amidoalkynyl, amidocarbocyclyl, amidoaryl, amidoheterocyclyl, amidoheteroaryl, amidoacyl, amidoaralkyl, formylalkyl, formylalkenyl, formylalkynyl, formylcarbocyclyl, formylaryl, formylheterocyclyl, formylheteroaryl, formylacyl, formylaralkyl, acylalkyl, acylalkenyl, acylalkynyl, acylcarbocyclyl, acylaryl, acylheterocyclyl, acylheteroaryl, acylacyl, acylaralkyl, sulfoxidealkyl, sulfoxidealkenyl, sulfoxidealkynyl, sulfoxidecarbocyclyl, sulfoxidearyl, sulfoxideheterocyclyl, sulfoxideheteroaryl, sulfoxideacyl, sulfoxidearalkyl, sulfonylalkyl, sulfonylalkenyl, sulfonylalkynyl, sulfonylcarbocyclyl, sulfonylaryl, sulfonylheterocyclyl, sulfonylheteroaryl, sulfonylacyl, sulfonylaralkyl, sulfonamidoalkyl, sulfonamidoalkenyl, sulfonamidoalkynyl, sulfonamidocarbocyclyl, sulfonamidoaryl, sulfonamidoheterocyclyl, sulfonamidoheteroaryl, sulfonamidoacyl, sulfonamidoaralkyl, nitroalkyl, nitroalkenyl, nitroalkynyl, nitrocarbocyclyl, nitroaryl, nitroheterocyclyl, nitroheteroaryl, nitroacyl, nitroaralkyl, cyano, sulfate and phosphate groups.

In some embodiments, it may be desirable that a group (for example the R group) is optionally substituted with a polymer chain. An example of such a polymer chain includes a polyester, polyurethane, or copolymers thereof. Such a polymer chain may, or may not, have one or more drugs appended thereto. For example, the R group of the formulae disclosed herein may be substituted with a polymer chain. The skilled worker will recognise that the R group may therefore represent a point of branching of the polymer backbone within the drug polymer conjugate of the present invention. If R is substituted with a polymer chain, that polymer chain should also be bioerodible and not contain any repeat units that are coupled with a non-bioerodible moiety as described herein.

Preferred optional substituents include the aforementioned reactive functional groups or moieties, polymer chains and alkyl, (e.g. C₁₋₆ alkyl such as methyl, ethyl, propyl, butyl, cyclopropyl, cyclobutyl, cyclopentyl or cyclohexyl), hydroxyalkyl (e.g. hydroxymethyl, hydroxyethyl, hydroxypropyl), alkoxyalkyl (e.g. methoxymethyl, methoxyethyl, methoxypropyl, ethoxymethyl, ethoxyethyl, ethoxypropyl etc) alkoxy (e.g. C₁₋₆ alkoxy such as methoxy, ethoxy, propoxy, butoxy, cyclopropoxy, cyclobutoxy), halo, trifluoromethyl, trichloromethyl, tribromomethyl, hydroxy, phenyl (which itself may be further substituted e.g., by C₁₋₆ alkyl, halo, hydroxy, hydroxyC₁₋₆ alkyl, C₁₋₆ alkoxy, haloC₁₋₆alkyl, cyano, nitro OC(O)C₁₋₆ alkyl, and amino), benzyl (wherein benzyl itself may be further substituted e.g., by C₁₋₆ alkyl, halo, hydroxy, hydroxyC₁₋₆alkyl, C₁₋₆ alkoxy, haloC₁₋₆ alkyl, cyano, nitro OC(O)C₁₋₆ alkyl, and amino), phenoxy (wherein phenyl itself may be further substituted e.g., by C₁₋₆ alkyl, halo, hydroxy, hydroxyC₁₋₆ alkyl, C₁₋₆ alkoxy, haloC₁₋₆ alkyl, cyano, nitro OC(O)C₁₋₆ alkyl, and amino), benzyloxy (wherein benzyl itself may be further substituted e.g., by C₁₋₆ alkyl, halo, hydroxy, hydroxyC₁₋₆ alkyl, C₁₋₆ alkoxy, haloC₁₋₆ alkyl, cyano, nitro OC(O)C₁₋₆ alkyl, and amino), amino, alkylamino (e.g. C₁₋₆ alkyl, such as methylamino, ethylamino, propylamino etc), dialkylamino (e.g. C₁₋₆ alkyl, such as dimethylamino, diethylamino, dipropylamino), acylamino (e.g. NHC(O)CH₃), phenylamino (wherein phenyl itself may be further substituted e.g., by C₁₋₆ alkyl, halo, hydroxy hydroxyC₁₋₆alkyl, C₁₋₆ alkoxy, haloC₁₋₆ alkyl, cyano, nitro OC(O)C₁₋₆ alkyl, and amino), nitro, formyl, —C(O)-alkyl (e.g. C₁₋₆ alkyl, such as acetyl), O—C(O)-alkyl (e.g. C₁₋₆alkyl, such as acetyloxy), benzoyl (wherein the phenyl group itself may be further substituted e.g., by C₁₋₆ alkyl, halo, hydroxy hydroxyC₁₋₆ alkyl, C₁₋₆ alkoxy, haloC₁₋₆ alkyl, cyano, nitro OC(O)C₁₋₆alkyl, and amino), replacement of CH₂ with C═O, CO₂H, CO₂alkyl (e.g. C₁₋₄ alkyl such as methyl ester, ethyl ester, propyl ester, butyl ester), CO₂-phenyl (wherein phenyl itself may be further substituted e.g., by C₁₋₆ alkyl, halo, hydroxy, hydroxy C₁₋₆ alkyl, C₁₋₆ alkoxy, halo C₁₋₆ alkyl, cyano, nitro OC(O)C₁₋₆ alkyl, and amino), CONH₂, CONHphenyl (wherein phenyl itself may be further substituted e.g., by C₁₋₆ alkyl, halo, hydroxy, hydroxy C₁₋₆ alkyl, C₁₋₆ alkoxy, halo C₁₋₆ alkyl, cyano, nitro OC(O)C₁₋₆ alkyl, and amino), CONHbenzyl (wherein benzyl itself may be further substituted e.g., by C₁₋₆ alkyl, halo, hydroxy C₁₋₆ alkyl, C₁₋₆ alkoxy, halo C₁₋₆ alkyl, cyano, nitro OC(O)C₁₋₆ alkyl, and amino), CONHalkyl (e.g. C₁₋₆ alkyl such as methyl ester, ethyl ester, propyl ester, butyl amide) CONHdialkyl (e.g. C₁₋₆ alkyl)aminoalkyl (e.g., HNC₁₋₆ alkyl-, C₁₋₆alkylHN—C₁₋₆alkyl- and (C₁₋₆ alkyl)₂N—C₁₋₆ alkyl-), thioalkyl (e.g., HS. C₁₋₆ alkyl-), carboxyalkyl (e.g., HO₂CC₁₋₆ alkyl-), carboxyesteralkyl (e.g., C₁₋₆ alkylO₂CC₁₋₆ alkyl-), amidoalkyl (e.g., H₂N(O)CC₁₋₆ alkyl-, H(C₁₋₆ alkyl)N(O)CC₁₋₆ alkyl-), formylalkyl (e.g., OHCC₁₋₆ alkyl-), acylalkyl (e.g., C₁₋₆ alkyl(O)CC₁₋₆ alkyl-), nitroalkyl (e.g., O₂NC₁₋₆ alkyl-), sulfoxidealkyl (e.g., R³(O)SC₁₋₆ alkyl, such as C₁₋₆ alkyl(O)SC₁₋₆ alkyl-), sulfonylalkyl (e.g., R³(O)₂SC₁₋₆ alkyl-such as C₁₋₆ alkyl(O)₂SC₁₋₆ alkyl-), sulfonamidoalkyl (e.g., ₂HRN(O)SC₁₋₆ alkyl, H(C₁₋₆ alkyl)N(O)SC₁₋₆ alkyl-).

As used herein, the term “aliphatic”, used either alone or in compound words denotes straight chain saturated and unsaturated hydrocarbyl. Examples of aliphatic groups include alkanes, alkenes, and alkynes.

As used herein, the term “alicyclic”, used either alone or in compound words denotes cyclic non-aromatic hydrocarbyl. An example of an alicyclic group is cyclohexane.

As used herein, the term “alkyl”, used either alone or in compound words denotes straight chain, branched or cyclic alkyl, for example C₁₋₄₀ alkyl, or C₁₋₂₀ or C₁₋₁₀. Examples of straight chain and branched alkyl include methyl, ethyl, n-propyl, isopropyl, n-butyl, sec-butyl, t-butyl, n-pentyl, 1,2-dimethylpropyl, 1,1-dimethyl-propyl, hexyl, 4-methylpentyl, 1-methylpentyl, 2-methylpentyl, 3-methylpentyl, I,I-dimethylbutyl, 2,2-dimethylbutyl, 3,3-dimethylbutyl, 1,2-dimethylbutyl, 1,3-dimethylbutyl, 1,2,2-trimethylpropyl, 1,1,2-trimethylpropyl, heptyl, 5-methylhexyl, 1-methylhexyl, 2,2-dimethylpentyl, 3,3-dimethylpentyl, 4,4-dimethylpentyl, 1,2-dimethylpentyl, 1,3-dimethylpentyl, 1,4-dimethyl-pentyl, 1,2,3-trimethylbutyl, 1,1,2-trimethylbutyl, 1,1,3-trimethylbutyl, octyl, 6-methylheptyl, 1-methylheptyl, 1,1,3,3-tetramethylbutyl, nonyl, 1-, 2-, 3-, 4-, 5-, 6- or 7-methyloctyl, 1-, 2-, 3-, 4- or 5-ethylheptyl, 1-, 2- or 3-propylhexyl, decyl, 1-, 2-, 3-, 4-, 5-, 6-, 7- and 8-methylnonyl, 1-, 2-, 3-, 4-, 5- or 6-ethyloctyl, 1-, 2-, 3- or 4-propylheptyl, undecyl, 1-, 2-, 3-, 4-, 5-, 6-, 7-, 8- or 9-methyldecyl, 1-, 2-, 3-, 4-, 5-, 6- or 7-ethylnonyl, 1-, 2-, 3-, 4- or 5-propyloctyl, 1-, 2- or 3-butylheptyl, 1-pentylhexyl, dodecyl, 1-, 2-, 3-, 4-, 5-, 6-, 7-, 8-, 9- or 10-methylundecyl, 1-, 2-, 3-, 4-, 5-, 6-, 7- or 8-ethyldecyl, 1-, 2-, 3-, 4-, 5- or 6-propylnonyl, 1-, 2-, 3- or 4-butyloctyl, 1-2-pentylheptyl, tridecyl, tetradecyl, pentadecyl, hexadecyl, heptadecyl, octadecyl, nonoadecyl, eicosyl and the like. Examples of cyclic alkyl include mono- or polycyclic alkyl groups such as cyclopropyl, cyclobutyl, cyclopentyl, cyclohexyl, cycloheptyl, cyclooctyl, cyclononyl, cyclodecyl and the like. Where an alkyl group is referred to generally as “propyl”, butyl” etc, it will be understood that this can refer to any of straight, branched and cyclic isomers where appropriate. An alkyl group may be optionally substituted by one or more optional substituents as herein defined.

As used herein, term “alkenyl” denotes groups formed from straight chain, branched or cyclic hydrocarbon residues containing at least one carbon to carbon double bond including ethylenically mono-, di- or polyunsaturated alkyl or cycloalkyl groups as previously defined, for example C₂₋₄₀ alkenyl, or C₂₋₂₀ or C₂₋₁₀. Thus, alkenyl is intended to include propenyl, butylenyl, pentenyl, hexaenyl, heptaenyl, octaenyl, nonaenyl, decenyl, undecenyl, dodecenyl, tridecenyl, tetradecenyl, pentadecenyl, hexadecenyl, heptadecenyl, octadecenyl, nondecenyl, eicosenyl hydrocarbon groups with one or more carbon to carbon double bonds. Examples of alkenyl include vinyl, allyl, 1-methylvinyl, butenyl, iso-butenyl, 3-methyl-2-butenyl, 1-pentenyl, cyclopentenyl, 1-methyl-cyclopentenyl, 1-hexenyl, 3-hexenyl, cyclohexenyl, 1-heptenyl, 3-heptenyl, 1-octenyl, cyclooctenyl, 1-nonenyl, 2-nonenyl, 3-nonenyl, 1-decenyl, 3-decenyl, 1,3-butadienyl, 1,4-pentadienyl, 1,3-cyclopentadienyl, 1,3-hexadienyl, 1,4-hexadienyl, 1,3-cyclohexadienyl, 1,4-cyclohexadienyl, 1,3-cycloheptadienyl, 1,3,5-cycloheptatrienyl and 1,3,5,7-cyclooctatetraenyl. An alkenyl group may be optionally substituted by one or more optional substituents as herein defined.

As used herein the term “alkynyl” denotes groups formed from straight chain, branched or cyclic hydrocarbon residues containing at least one carbon-carbon triple bond including ethylenically mono-, di- or polyunsaturated alkyl or cycloalkyl groups as previously defined, for example, C₂₋₄₀ alkenyl, or C₂₋₂₀ or C₂₋₁₀. Thus, alkynyl is intended to include propynyl, butylynyl, pentynyl, hexaynyl, heptaynyl, octaynyl, nonaynyl, decynyl, undecynyl, dodecynyl, tridecynyl, tetradecynyl, pentadecynyl, hexadecynyl, heptadecynyl, octadecynyl, nondecynyl, eicosynyl hydrocarbon groups with one or more carbon to carbon triple bonds. Examples of alkynyl include ethynyl, 1-propynyl, 2-propynyl, and butynyl isomers, and pentynyl isomers.

An alkynyl group may be optionally substituted by one or more optional substituents as herein defined.

An alkenyl group may comprise a carbon to carbon triple bond and an alkynyl group may comprise a carbon to carbon double bond (i.e. so called ene-yne or yne-ene groups).

As used herein, the term “aryl” (or “carboaryl)” denotes any of single, polynuclear, conjugated and fused residues of aromatic hydrocarbon ring systems. Examples of aryl include phenyl, biphenyl, terphenyl, quaterphenyl, naphthyl, tetrahydronaphthyl, anthracenyl, dihydroanthracenyl, benzanthracenyl, dibenzanthracenyl, phenanthrenyl, fluorenyl, pyrenyl, idenyl, azulenyl, chrysenyl. Preferred aryl include phenyl and naphthyl. An aryl group may be optionally substituted by one or more optional substituents as herein defined.

As used herein, the terms “alkylene”, “alkenylene”, and “arylene” are intended to denote the divalent forms of “alkyl”, “alkenyl”, and “aryl”, respectively, as herein defined.

The term “halogen” (“halo”) denotes fluorine, chlorine, bromine or iodine (fluoro, chloro, bromo or iodo). Preferred halogens are chlorine, bromine or iodine.

The term “carbocyclyl” includes any of non-aromatic monocyclic, polycyclic, fused or conjugated hydrocarbon residues, preferably C₃₋₂₀ (e.g. C₃₋₁₀ or C₃₋₈). The rings may be saturated, e.g. cycloalkyl, or may possess one or more double bonds (cycloalkenyl) and/or one or more triple bonds (cycloalkynyl). Particularly preferred carbocyclyl moieties are 5-6-membered or 9-10 membered ring systems. Suitable examples include cyclopropyl, cyclobutyl, cyclopentyl, cyclohexyl, cycloheptyl, cyclooctyl, cyclononyl, cyclodecyl, cyclopentenyl, cyclohexenyl, cyclooctenyl, cyclopentadienyl, cyclohexadienyl, cyclooctatetraenyl, indanyl, decalinyl and indenyl.

The term “heterocyclyl” when used alone or in compound words includes any of monocyclic, polycyclic, fused or conjugated hydrocarbon residues, preferably C₃₋₂₀ (e.g. C₃₋₁₀ or C₃₋₈) wherein one or more carbon atoms are replaced by a heteroatom so as to provide a non-aromatic residue. Suitable heteroatoms include O, N, S, P and Se, particularly O, N and S. Where two or more carbon atoms are replaced, this may be by two or more of the same heteroatom or by different heteroatoms. The heterocyclyl group may be saturated or partially unsaturated, i.e. possess one or more double bonds. Particularly preferred heterocyclyl are 5-6 and 9-10 membered heterocyclyl. Suitable examples of heterocyclyl groups may include azridinyl, oxiranyl, thiiranyl, azetidinyl, oxetanyl, thietanyl, 2H-pyrrolyl, pyrrolidinyl, pyrrolinyl, piperidyl, piperazinyl, morpholinyl, indolinyl, imidazolidinyl, imidazolinyl, pyrazolidinyl, thiomorpholinyl, dioxanyl, tetrahydrofuranyl, tetrahydropyranyl, tetrahydropyrrolyl, tetrahydrothiophenyl, pyrazolinyl, dioxalanyl, thiazolidinyl, isoxazolidinyl, dihydropyranyl, oxazinyl, thiazinyl, thiomorpholinyl, oxathianyl, dithianyl, trioxanyl, thiadiazinyl, dithiazinyl, trithianyl, azepinyl, oxepinyl, thiepinyl, indenyl, indanyl, 3H-indolyl, isoindolinyl, 4H-quinolazinyl, chromenyl, chromanyl, isochromanyl, pyranyl and dihydropyranyl.

The term “heteroaryl” includes any of monocyclic, polycyclic, fused or conjugated hydrocarbon residues, wherein one or more carbon atoms are replaced by a heteroatom so as to provide an aromatic residue. Preferred heteroaryl have 3-20 ring atoms, e.g. 3-10. Particularly preferred heteroaryl are 5-6 and 9-10 membered bicyclic ring systems. Suitable heteroatoms include, O, N, S, P and Se, particularly O, N and S. Where two or more carbon atoms are replaced, this may be by two or more of the same heteroatom or by different heteroatoms. Suitable examples of heteroaryl groups may include pyridyl, pyrrolyl, thienyl, imidazolyl, furanyl, benzothienyl, isobenzothienyl, benzofuranyl, isobenzofuranyl, indolyl, isoindolyl, pyrazolyl, pyrazinyl, pyrimidinyl, pyridazinyl, indolizinyl, quinolyl, isoquinolyl, phthalazinyl, 1,5-naphthyridinyl, quinozalinyl, quinazolinyl, quinolinyl, oxazolyl, thiazolyl, isothiazolyl, isoxazolyl, triazolyl, oxadialzolyl, oxatriazolyl, triazinyl, and furazanyl.

The term “acyl” either alone or in compound words denotes a group containing the agent C═O (and not being a carboxylic acid, ester or amide) Preferred acyl includes C(O)—R^{x}, wherein R^{x} is hydrogen or an alkyl, alkenyl, alkynyl, aryl, heteroaryl, carbocyclyl, or heterocyclyl residue. Examples of acyl include formyl, straight chain or branched alkanoyl (e.g. C₁₋₂₀) such as, acetyl, propanoyl, butanoyl, 2-methylpropanoyl, pentanoyl, 2,2-dimethylpropanoyl, hexanoyl, heptanoyl, octanoyl, nonanoyl, decanoyl, undecanoyl, dodecanoyl, tridecanoyl, tetradecanoyl, pentadecanoyl, hexadecanoyl, heptadecanoyl, octadecanoyl, nonadecanoyl and icosanoyl; cycloalkylcarbonyl such as cyclopropylcarbonyl cyclobutylcarbonyl, cyclopentylcarbonyl and cyclohexylcarbonyl; aroyl such as benzoyl, toluoyl and naphthoyl; aralkanoyl such as phenylalkanoyl (e.g. phenylacetyl, phenylpropanoyl, phenylbutanoyl, phenylisobutylyl, phenylpentanoyl and phenylhexanoyl) and naphthylalkanoyl (e.g. naphthylacetyl, naphthylpropanoyl and naphthylbutanoyl]; aralkenoyl such as phenylalkenoyl (e.g. phenylpropenoyl, phenylbutenoyl, phenylmethacryloyl, phenylpentenoyl and phenylhexenoyl and naphthylalkenoyl (e.g. naphthylpropenoyl, naphthylbutenoyl and naphthylpentenoyl); aryloxyalkanoyl such as phenoxyacetyl and phenoxypropionyl; aryithiocarbamoyl such as phenylthiocarbamoyl; arylglyoxyloyl such as phenylglyoxyloyl and naphthylglyoxyloyl; arylsulfonyl such as phenylsulfonyl and napthylsulfonyl; heterocycliccarbonyl; heterocyclicalkanoyl such as thienylacetyl, thienylpropanoyl, thienylbutanoyl, thienylpentanoyl, thienylhexanoyl, thiazolylacetyl, thiadiazolylacetyl and tetrazolylacetyl; heterocyclicalkenoyl such as heterocyclicpropenoyl, heterocyclicbutenoyl, heterocyclicpentenoyl and heterocyclichexenoyl; and heterocyclicglyoxyloyl such as thiazolyglyoxyloyl and thienylglyoxyloyl. The R^{x} residue may be optionally substituted as described herein.

The term “sulfoxide”, either alone or in a compound word, refers to a group —S(O)R^{y} wherein R^{y} is selected from hydrogen, alkyl, alkenyl, alkynyl, aryl, heteroaryl, heterocyclyl, carbocyclyl, and aralkyl. Examples of preferred R^{y} include C₁₋₂₀alkyl, phenyl and benzyl.

The term “sulfonyl”, either alone or in a compound word, refers to a group S(O)₂—R^{y}, wherein R^{y} is selected from hydrogen, alkyl, alkenyl, alkynyl, aryl, heteroaryl, heterocyclyl, carbocyclyl and aralkyl. Examples of preferred R^{y} include C₁₋₂₀alkyl, phenyl and benzyl.

The term “sulfonamide”, either alone or in a compound word, refers to a group S(O)NR^{y}R^{y} wherein each R^{y} is independently selected from hydrogen, alkyl, alkenyl, alkynyl, aryl, heteroaryl, heterocyclyl, carbocyclyl, and aralkyl. Examples of preferred R^{y} include C₁₋₂₀alkyl, phenyl and benzyl. In a preferred embodiment at least one R^{y} is hydrogen. In another form, both R^{y} are hydrogen.

The term, “amino” is used here in its broadest sense as understood in the art and includes groups of the formula NR^{A}R^{B} wherein R^{A} and R^{B} may be any independently selected from hydrogen, alkyl, alkenyl, alkynyl, aryl, carbocyclyl, heteroaryl, heterocyclyl, aralkyl, and acyl. R^{A} and R^{B}, together with the nitrogen to which they are attached, may also form a monocyclic, or polycyclic ring system e.g. a 3-10 membered ring, particularly, 5-6 and 9-10 membered systems. Examples of “amino” include NH₂, NHalkyl (e.g. C₁₋₂₀alkyl), NHaryl (e.g. NHphenyl), NHaralkyl (e.g. NHbenzyl), NHacyl (e.g. NHC(O)C₁₋₂₀alkyl, NHC(O)phenyl), Nalkylalkyl (wherein each alkyl, for example C₁₋₂₀, may be the same or different) and 5 or 6 membered rings, optionally containing one or more same or different heteroatoms (e.g. O, N and S).

The term “amido” is used here in its broadest sense as understood in the art and includes groups having the formula C(O)NR^{A}R^{B}, wherein R^{A} and R^{B} are as defined as above. Examples of amido include C(O)NH₂, C(O)NHalkyl (e.g. C₁₋₂₀alkyl), C(O)NHaryl (e.g. C(O)NHphenyl), C(O)NHaralkyl (e.g. C(O)NHbenzyl), C(O)NHacyl (e.g. C(O)NHC(O)C₁₋₂₀alkyl, C(O)NHC(O)phenyl), C(O)Nalkylalkyl (wherein each alkyl, for example C₁₋₂₀, may be the same or different) and 5 or 6 membered rings, optionally containing one or more same or different heteroatoms (e.g. O, N and S).

The term “carboxy ester” is used here in its broadest sense as understood in the art and includes groups having the formula CO₂R^{z}, wherein R^{z} may be selected from groups including alkyl, alkenyl, alkynyl, aryl, carbocyclyl, heteroaryl, heterocyclyl, aralkyl, and acyl. Examples of carboxy ester include CO₂C₁₋₂₀alkyl, CO₂aryl (e.g. CO₂-phenyl), CO₂aralkyl (e.g. CO₂ benzyl).

The term “heteroatom” or “hetero” as used herein in its broadest sense refers to any atom other than a carbon atom which may be a member of a cyclic organic group. Particular examples of heteroatoms include nitrogen, oxygen, sulfur, phosphorous, boron, silicon, selenium and tellurium, more particularly nitrogen, oxygen and sulfur.

It is understood that the compounds of the present invention (including monomers and polymers) may exist in one or more stereoisomeric forms (eg enantiomers, diastereomers). The present invention includes within its scope all of these stereoisomeric forms either isolated (in for example enantiomeric isolation), or in combination (including racemic mixtures).

The invention will now be described with reference to the following non-limiting examples:

### EXAMPLES

### Experimental Procedures

### Procedure 1

### General Procedure for HBTU Coupling

A solution of prostaglandin free acid (1) (1.0 eq.) in anhydrous THF is added dropwise into a stirred solution of HBTU (˜1.2 eq.), the alcohol/glycerol derivative (˜1.6 eq.) and triethylamine (˜4.3 eq.) in anhydrous THF under nitrogen atmosphere. The mixture was stirred at room temperature for 3 days, with the exclusion of light, or until the reaction is complete. The reaction was quenched with 1M aqueous citric acid and extracted with ethyl acetate. The organic phase was then washed with saturated aqueous sodium hydrogen carbonate, followed by brine. The organic phase was then dried over Na₂SO₄, filtered, concentrated and dried in vacuo.

### Procedure 2

### General Procedure for Benzylidene Deprotection

Benzylidene protected derivative (˜1 mmol) is dissolved in 80% acetic acid (20 mL) and stirred at room temperature for 48 h or until the reaction is complete. The solvent is removed under reduced pressure and the residue is washed with toluene and dried in vacuo.

### Procedure 3

### General Procedure for Formation of 9,11-Boronated Prostaglandin

N-butylboronic acid (˜1.1 eq.) is added to a solution of prostaglandin derivative (1 eq.) in anhydrous DCM. The mixture is heated at 45° C. for 1 h under nitrogen atmosphere. Solvent is removed and dried in vacuo. Additional anhydrous DCM is added and removed in vacuo for a further 3 h. The residue is further heated in anhydrous DCM (10 mL) at 45° C. for 16 h and the solvent is removed under reduced pressure, to provide the 9,11-Boronated Prostaglandin.

### Procedure 4

### General Procedure for Formation of 15-O-Ester Prostaglandin

A mixture of boronate prostaglandin (1 eq.), 4-nitrophenyl 2-phenyl-1,3-dioxane-5-carboxylate (˜1.5 eq.) and DMAP (˜3.8 eq.) in anhydrous DCM was stirred at room temperature for 48 h or until the reaction is complete. The solvent was removed in vacuo to give a residue, which is dissolved in methanol and stirred at room temperature for a further 16 h.

### Polymerisation Method A:

An isocyanate (˜1.15 eq.) is added to a solution of prostaglandin-monomer conjugate (1 eq.) and dibutyltindilaurate (DBTDL) (catalytic, ˜0.1 eq.) in anhydrous THF under nitrogen atmosphere. The reaction mixture is stirred at room temperature for 24 h and the solvent is removed under reduced pressure. The residue is dissolved in DCM and added dropwise to a stirred solution of diethyl ether. The mixture is stirred at room temperature for 1 h and the solvent is decanted. The residue is washed with diethyl ether and then dried in vacuo to obtain the desired polymer drug conjugate.

### Polymerisation Method B:

An isocyanate (˜1.15 eq.) is added to a solution of prostaglandin-monomer conjugate (1 eq.) and dibutyltindilaurate (DBTDL) (catalytic, ˜0.1 eq.) in anhydrous THF under nitrogen atmosphere. The reaction mixture is heated to 45° C. and stirred for 24 h under nitrogen atmosphere. The reaction mixture is allowed to cool to room temperature and the solvent is removed under reduced pressure. The residue is dissolved in DCM and added dropwise to a stirred solution of diethyl ether. The mixture is stirred at room temperature for 1 h and the solvent is decanted. The residue is washed with diethyl ether and then dried in vacuo to obtain the desired polymer drug conjugate.

### Polymerisation Method C:

This method introduces a hydrophilic component in the polymer backbone the hydrophilic component is introduced by copolymeising a hydrophilic monomer with the drug-monomer conjugate.

An isocyanate (˜1.15 eq.) is added to a solution of prostaglandin-monomer conjugate (X eq.) and a desired hydrophilic co-monomer (Y eq.) in THF, such that the combined amounts of prostaglandin monomer and hydrophilic co-monomer is 1.0 eq. (X+Y=1.0). Dibutyltindilaurate (DBTDL) (catalytic, ˜0.1 eq.) is added and the reaction mixture heated to 45° C. and stirred for 24 h under nitrogen atmosphere. The reaction mixture is allowed to cool to room temperature and the solvent is removed under reduced pressure. The residue is dissolved in DCM and added dropwise to a stirred solution of diethyl ether. The mixture is stirred at room temperature for 1 h and the solvent is decanted. The residue is washed with diethyl ether and the dried in vacuo to obtain the desired polymer drug conjugate.

### Polymerisation Method D:

This method introduces a hydrophilic component by blending a hydrophilic polymer with a polymer drug conjugate. The polymer drug conjugate is preformed according to any one of procedures A to C and then dissolved in THF. A hydrophilic polymer is added and the mixture is stirred for 1 h. The solvent is removed under reduced pressure and the process is repeated to provide a polymer drug conjugate with a co-monomer blend.

### Synthesis of Drug-Monomer Conjugates

### Latanoprost Free Acid (1)

Synthesis of (Z)-7-((1R,2R,3R,5S)-3,5-Dihydroxy-2-((R)-3-hydroxy-5-phenylpentyl)cyclopentyl)hept-5-enoic acid, latanoprost free acid (1) was carried out according to the literature, Eur. J. Org. Chem., 2007, 689-703.

### Fluprostenol-Travoprost Free Acid (8)

Synthesis of (Z)-isopropyl 7-((1R,2R,3R,5S)-3,5-dihydroxy-2-((R,E)-3-hydroxy-4-(3-(trifluoromethyl)phenoxy)but-1-en-1-yl)cyclopentyl)hept-5-enoate, travoprost free acid (8) was carried out according to the literature, Lett. Org. Chem. 2011, 8, 234-241.

### Example 1

### (Z)-3-Hydroxy-2-(hydroxymethyl)-2-methylpropyl 7-((1R,2R,3R,5S)-3,5-dihydroxy-2-((R)-3-hydroxy-5-phenylpentyl)cyclopentyl)hept-5-enoate (2)

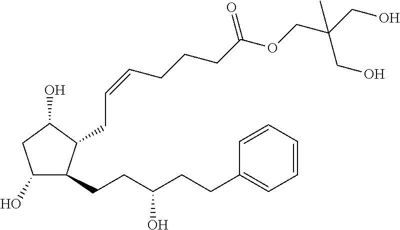 [SMILES: CC(CO)(CO)COC(=O)CCC/C=C\C[C@@H]1[C@@H](CC[C@@H](O)CCc2ccccc2)[C@H](O)C[C@@H]1O]

The general procedure for HBTU coupling (Procedure 1) was followed using latanoprost free acid (1) (407.1 mg, 1.0 mmol), HBTU (440.3 mg, 1.2 mmol), 1,1,1-trishydroxymethyl ethane (187.9 mg, 1.6 mmol) and triethylamine (0.60 mL, 4.3 mmol) in anhydrous THF. The residue was chromatographed (SiO₂, MeOH—CHCl₃, 10:90) to give the title compound (2) (322.0 mg, 63% yield) as a clear colourless oil. ESI-MS: m/z 538 ([M+2Na]⁺); ¹H NMR (400 MHz, CDCl₃) δ (ppm): 7.34-7.16 (m, 3H), 7.16-7.00 (m, 2H), 5.43-5.36 (m, 1H), 5.35-5.18 (m, 1H), 4.16-3.97 (m, 2H), 3.89-3.74 (m, 1H), 3.61-3.51 (m, 1H), 3.45 (s, 3H), 3.41-3.31 (m, 4H), 2.80-2.65 (m, 2H), 2.65-2.46 (m, 2H), 2.40-1.96 (m, 5H), 1.91-1.35 (m, 8H), 1.35-1.20 (m, 2H), 0.77 (s, 2H).

### Example 2

### (Z)-1,3-Dihydroxypropan-2-yl 7-((1R,2R,3R,5S)-3,5-dihydroxy-2-((R)-3-hydroxy-5-phenylpentyl)cyclopentyl)hept-5-enoate (5)

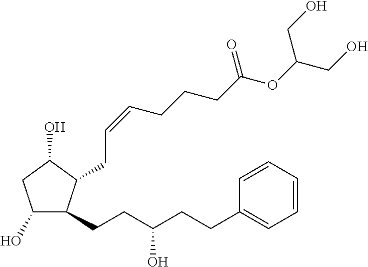 [SMILES: O=C(CCC/C=C\C[C@@H]1[C@@H](CC[C@@H](O)CCc2ccccc2)[C@H](O)C[C@@H]1O)OC(CO)CO]

The general procedure for HBTU coupling (Procedure 1) was followed, using latanoprost free acid (1) (528.2 mg, 1.35 mmol), 1,3-benzylidene glycerol (309.0 mg, 1.71 mmol), HBTU (564.5 mg, 1.49 mmol) and triethylamine (0.8 mL, 5.75 mmol) in anhydrous DCM. The crude material was chromatographed (SiO₂, EtOAc, 100%) to give the benzylidene ester (3) (412.3 mg, 55% yield) as a clear colourless oil. ESI-MS: m/z 575 ([M+Na]⁺); ¹H NMR (400 MHz, CDCl₃) δ (ppm): 7.49-7.37 (m, 2H), 7.37-7.24 (m, 3H), 7.24-7.16 (m, 2H), 7.16-7.03 (m, 3H), 5.48 (s, 1H), 5.41-5.31 (m, 4H), 4.70-4.57 (m, 1H), 4.26-3.94 (m, 5H), 3.90-3.69 (m, 1H), 3.81-3.82 (m, 1H), 2.77-2.64 (m, 1H), 2.62-2.54 (m, 1H), 2.38 (td, J=7.2, 1.2 Hz, 3H), 2.30-1.98 (m, 6H), 1.82-1.35 (m, 10H), 1.35-1.13 (m, 2H).

The general procedure for benzylidene deprotection (Procedure 2) was followed using the benzylidene ester (3) (412.3 mg, 0.75 mmol) in 80% acetic acid (20 mL). The crude product was chromatographed (SiO₂, MeOH:CHCl₃, 10:90%) to give the title compound (5) (317.5 mg, 92% yield) as a clear colourless oil. ESI-MS: m/z 510 ([M+2Na]⁺); ¹H NMR (400 MHz, CDCl₃) δ (ppm): 7.26-7.15 (m, 2H), 7.15-7.02 (m, 3H), 5.45-5.17 (m, 2H), 4.83 (p, J=4.8 Hz, 1H), 4.21-3.95 (m, 2H), 3.95-3.75 (m, 2H), 3.75-3.13 (m, 8H), 2.82-2.46 (m, 2H), 2.39-2.16 (m, 2H), 2.16-1.91 (m, 3H), 1.91-1.78 (m, 1H), 1.78-0.96 (m, 12H).

### Example 3

### 1,3-Dihydroxypropan-2-yl 4-(((Z)-7-((1R,2R,3R,5S)-3,5-dihydroxy-2-((R)-3-hydroxy-5-phenylpentyl)cyclopentyl)hept-5-enoyl)oxy)benzoate (6)

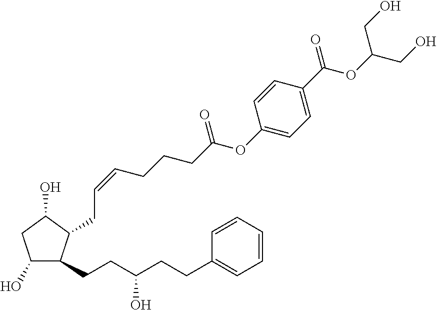 [SMILES: O=C(CCC/C=C\C[C@@H]1[C@@H](CC[C@@H](O)CCc2ccccc2)[C@H](O)C[C@@H]1O)Oc1ccc(C(=O)OC(CO)CO)cc1]

The general procedure for HBTU coupling (Procedure 1) was followed, using latanoprost free acid (1) (234.1 mg, 0.60 mmol), 2-phenyl-1,3-dioxan-5-yl 4-hydroxybenzoate (361.5 mg, 1.20 mmol), HBTU (251.4 mg, 0.66 mmol) and triethylamine (0.5 mL 3.59 mmol) in anhydrous DCM (15 mL). The crude material was chromatographed (SiO₂, EtOAc, 100%) to give the benzylidene ester (4) (258.7 mg, 63% yield) as a clear colourless oil. ESI-MS: m/z 695 ([M+Na]⁺); ¹H NMR (400 MHz, CDCl₃) δ (ppm): 8.17-8.04 (m, 2H), 7.55-7.40 (m, 2H), 7.40-7.25 (m, 3H), 7.25-7.16 (m, 2H), 7.16-7.02 (m, 5H), 5.55 (s, 1H), 5.50-5.26 (m, 2H), 4.94-4.79 (m, 1H), 4.41-4.12 (m, 4H), 4.12-3.97 (m, 1H), 3.93-3.79 (m, 1H), 3.65-3.49 (m, 1H), 2.73-2.55 (m, 2H), 2.43-2.06 (m, 5H), 1.87-1.38 (m, 13H), 1.38-1.22 (m, 2H).

The general procedure for benzylidene deprotection (Procedure 2) was followed, using the benzylidene ester (4) (196.9 mg, 0.29 mmol) in 80% acetic acid (5 mL). The crude material was chromatographed (SiO₂, MeOH:CHCl₃, 10:90%) to give the title compound (6) (122.9 mg, 72% yield) as a clear colourless oil. ESI-MS: m/z 630 ([M+2Na]⁺); ¹H NMR (400 MHz, CDCl₃) δ (ppm): 8.08-7.95 (m, 2H), 7.28-7.15 (m, 2H), 7.15-7.02 (m, 5H), 5.39 (dtd, J=18.1, 10.9, 7.2 Hz, 2H), 5.04 (p, J=4.7 Hz, 1H), 4.13-3.98 (m, 1H), 3.92-3.75 (m, 5H), 3.59-3.46 (m, 1H), 3.40 (s, 1H), 2.74-2.44 (m, 5H), 2.36-2.03 (m, 5H), 1.86-1.32 (m, 12H), 1.32-1.19 (m, 2H).

### Example 4

### (Z)-3-hydroxy-2-(hydroxymethyl)-2-methylpropyl 7-((1R,2R,3R,5S)-3,5-dihydroxy-2-((R,E)-3-hydroxy-4-(3-(trifluoromethyl)phenoxy)but-1-en-1-yl)cyclopentyl)hept-5-enoate (24)

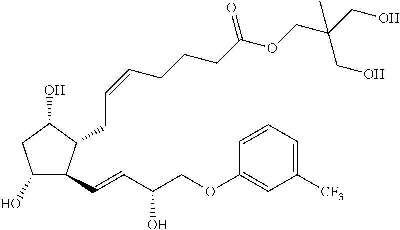 [SMILES: CC(CO)(CO)COC(=O)CCC/C=C\C[C@@H]1[C@@H](/C=C/[C@@H](O)COc2cccc(C(F)(F)F)c2)[C@H](O)C[C@@H]1O]

The general procedure for HBTU coupling (Procedure 1) was followed, using travoprost free acid (8) (410.1 mg, 0.89 mmol), 1,1,1-trishydroxymethyl ethane (167.0 mg, 1.39 mmol), HBTU (374.7 mg, 0.98 mmol) and triethylamine (0.55 mL, 3.95 mmol) in anhydrous DCM (15 mL) to give the title compound (24) (39 mg) as a clear colourless oil. ESI-MS: m/z 583 ([M+Na]⁺).

### Example 5

### (Z)-isopropyl 7-((1R,2R,3R,5S)-3,5-dihydroxy-2-((R)-3-((3-hydroxy-2-(hydroxymethyl)propanoyl)oxy)-5-phenylpentyl)cyclopentyl)hept-5-enoate (14)

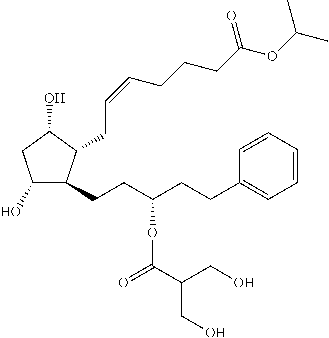 [SMILES: CC(C)OC(=O)CCC/C=C\C[C@@H]1[C@@H](CC[C@H](CCc2ccccc2)OC(=O)C(CO)CO)[C@H](O)C[C@@H]1O]

The general procedure for formation of 9,11-boronate latanoprost (Procedure 3) was followed, using latanoprost (222.0 mg, 0.51 mmol) and n-butylboronic acid (60.1 mg, 0.59 mmol) in anhydrous DCM (1 mL). The 9,11-boronate of latanoprost (9) was obtained as a clear colourless oil and used directly without further purification. ¹H NMR (400 MHz, CDCl₃) δ (ppm): 7.28-7.17 (m, 2H), 7.17-7.03 (m, 3H), 5.49-5.27 (m, 2H), 4.93 (ddd, J=15.2, 7.6, 4.9 Hz, 1H), 4.28-4.13 (m, 1H), 4.07-3.90 (m, 1H), 3.65-3.46 (m, 1H), 2.78-2.67 (m, 1H), 2.67-2.41 (m, 1H), 2.28-2.11 (m, 4H), 2.09-1.98 (m, 2H), 1.91-1.79 (m, 1H), 1.79-1.53 (m, 7H), 1.53-1.38 (m, 3H), 1.38-1.07 (m, 12H), 0.89-0.75 (m, 3H), 0.64-0.52 (m, 2H).

### Via Benzylidene Ester

The general procedure for the formation of 15-O-Ester Prostaglandin (Procedure 4) was followed, using 9,11-boronate of latanoprost (9) (116.6 mg, 0.23 mmol), 4-nitrophenyl 2-phenyl-1,3-dioxane-5-carboxylate (114.0 mg, 0.35 mmol) and DMAP (107.1 mg, 0.88 mmol) in anhydrous DCM (5 mL). The residue was dissolved in methanol (5 mL) and stirred for 16 h. The crude material was chromatographed (SiO₂, MeOH:CHCl₃, 10:90%) to give the benzylidene ester (11) (193.1 mg, 82% yield) as a clear colourless oil. ESI-MS: m/z 645 ([M+Na]⁺); ¹H NMR (400 MHz, CDCl₃) δ (ppm): 7.47-7.34 (m, 2H), 7.34-7.16 (m, 4H), 7.16-6.95 (m, 2H), 6.82-6.70 (m, 2H), 5.43-5.23 (m, 3H), 5.01-4.77 (m, 2H), 4.48-4.30 (m, 2H), 4.15 (s, 1H), 3.97 (s, 1H), 3.95-3.82 (m, 2H), 3.04 (tt, J=11.2, 4.8 Hz, 1H), 2.65-2.43 (m, 3H), 2.43-1.91 (m, 6H), 1.93-0.94 (m, 17H).

The general procedure for benzylidene deprotection (Procedure 2) was followed, using (11) (193.1 mg, 0.31 mmol) in 80% acetic acid (5 mL). The crude material was chromatographed (SiO₂, EtOAc, 100%) to give the title compound (14) (55.0 mg, 33% yield) as a clear colourless oil.

### Via 4-OMe Substituted Benzylidene Ester

The general procedure for the formation of 15-O-Ester Prostaglandin (Procedure 4) was followed, using 9,11-boronate of latanoprost (9) (526.1 mg, 1.05 mmol), 4-nitrophenyl 2-(4-methoxyphenyl)-1,3-dioxane-5-carboxylate (412.1 mg, 1.15 mmol) and DMAP (402.6 mg, 3.30 mmol) in anhydrous DCM (15 mL). The residue was dissolved in methanol (10 mL) and stirred for 16 h. The crude material was chromatographed (SiO₂, EtOAc:Hexane, 70:30%) to give the benzylidene ester (12) (444.2 mg, 64% yield) as a clear colourless oil. ESI-MS: m/z 676 ([M+Na]⁺); ¹H NMR (400 MHz, CDCl₃) δ (ppm): 7.37-7.28 (m, 2H), 7.26-7.16 (m, 2H), 7.16-7.03 (m, 3H), 6.88-6.73 (m, 2H), 5.43-5.23 (m, 3H), 5.02-4.83 (m, 2H), 4.43-4.27 (m, 2H), 4.10 (s, 1H), 3.96-3.84 (m, 2H), 3.82 (s, 1H), 3.77-3.68 (m, 3H), 3.03 (tt, J=11.2, 4.8 Hz, 1H), 2.63-2.46 (m, 3H), 2.37 (s, 1H), 2.33-2.16 (m, 3H), 2.16-1.94 (m, 3H), 1.93-1.53 (m, 10H), 1.45-1.23 (m, 2H), 1.23-0.95 (m, 6H).

The general procedure for benzylidene deprotection (Procedure 2) was followed, using (12) (297.2 mg, 0.46 mmol) in 80% acetic acid (10 mL). The mixture was stirred at room temperature for 4 h. The crude material was chromatographed (SiO₂, EtOAc, 100%) to give the title compound (14) (146.9 mg, 60% yield) as a clear colourless oil. ESI-MS: m/z 580 ([M+2Na]⁺); ¹H NMR (400 MHz, CDCl₃) δ (ppm): 7.27-7.15 (m, 2H), 7.15-6.92 (m, 3H), 5.50-5.20 (m, 2H), 5.02-4.78 (m, 2H), 4.13-3.97 (m, 1H), 3.94-3.72 (m, 5H), 3.60-3.02 (bs, 3H), 2.75-2.41 (m, 4H), 2.29-2.15 (m, 3H), 2.15-1.50 (m, 12H), 1.50-1.34 (m, 1H), 1.31-1.01 (m, 8H).

### Example 6

### (Z)-isopropyl 7-((1R,2R,3R,5S)-3,5-dihydroxy-2-((R,E)-3-((3-hydroxy-2-(hydroxymethyl)propanoyl)oxy)-4-(3-(trifluoromethyl)phenoxy)but-1-en-1-yl)cyclopentyl)hept-5-enoate (15)

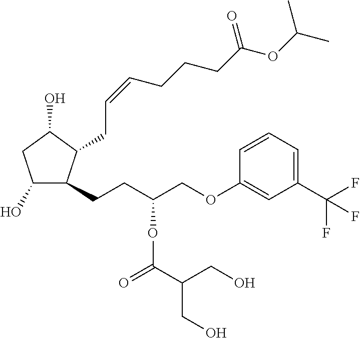 [SMILES: CC(C)OC(=O)CCC/C=C\C[C@@H]1[C@@H](CC[C@H](COc2cccc(C(F)(F)F)c2)OC(=O)C(CO)CO)[C@H](O)C[C@@H]1O]

The general procedure for 9,11-boronated prostaglandin (Procedure 3) was followed, using travoprost (55.1 mg, 0.11 mmol) and n-buytlboronic acid (13.6 mg, 0.13 mmol) in anhydrous DCM (1 mL). The 9,11-boronated travoprost (10) was obtained as a clear colourless oil and used directly without further purification. ¹H NMR δ: 7.37-7.27 (m, 1H), 7.22-7.10 (m, 1H), 7.10-7.04 (m, 1H), 7.04-6.92 (m, 1H), 5.75-5.48 (m, 2H), 5.45-5.24 (m, 2H), 5.03-4.78 (m, 1H), 4.65 (s, 1H), 4.53-4.38 (m, 1H), 4.27 (s, 1H), 4.13-4.00 (m, 1H), 4.00-3.76 (m, 2H), 2.51-2.32 (m, 2H), 2.31-2.11 (m, 4H), 2.11-1.97 (m, 2H), 1.97-1.83 (m, 1H), 1.83-1.67 (m, 2H), 1.67-1.56 (m, 2H), 1.54 (s, 1H), 1.37-1.05 (m, 8H), 0.91-0.68 (m, 3H), 0.67-0.49 (m, 2H).

The general procedure for the formation of 15-O-Ester of Prostaglandin (Procedure 4) was followed, using the 9,11-boronated travoprost (10) (62.4 mg, 0.11 mmol), 4-nitrophenyl 2-(4-methoxyphenyl)-1,3-dioxane-5-carboxylate (46.5 mg, 0.13 mmol) and DMAP (56.4 mg, 0.46 mmol) in anhydrous DCM (1 mL). The residue was dissolved in methanol (1 mL) and stirred for 16 h. The crude material was chromatographed (SiO₂, EtOAc:Hexane, 70:30%) to give the benzylidene ester (13) (59.9 mg, 75% yield) as a clear colourless oil. ESI-MS: m/z 765 ([M+2Na]⁺); ¹H NMR (400 MHz, CDCl₃) δ (ppm): 7.40-7.26 (m, 3H), 7.23-7.11 (m, 1H), 7.07-7.02 (m, 1H), 7.02-6.96 (m, 1H), 6.86-6.74 (m, 2H), 5.76-5.45 (m, 3H), 5.39-5.21 (m, 3H), 5.00-4.84 (m, 1H), 4.44-4.30 (m, 2H), 4.20-4.09 (m, 1H), 4.09-3.97 (m, 2H), 3.97-3.79 (m, 3H), 3.73 (s, 3H), 3.16-3.00 (m, 1H), 2.47-1.85 (m, 8H), 1.85-1.72 (m, 1H), 1.72-1.35 (m, 5H), 1.35-1.08 (m, 6H).

The general procedure for benzylidene deprotection (Procedure 2) was followed, using (13) (53.4 mg, 0.07 mmol) in 80% acetic acid (2 mL). The mixture was stirred at room temperature for 4 h. The crude mixture was passed through a thin layer of silica gel eluting with 70% ethyl acetate:hexanes, followed by 30% MeOH:CHCl₃. The title compound (15) (33.9 mg, quantitative yield) was obtained as a clear colourless oil. ESI-MS: m/z 647 ([M+2Na]⁺); ¹H NMR (400 MHz, CDCl₃) δ (ppm): 7.36-7.27 (m, 1H), 7.19-7.11 (m, 1H), 7.10-7.04 (m, 1H), 7.01 (dd, J=8.3, 2.3 Hz, 1H), 5.81-5.47 (m, 3H), 5.41-5.20 (m, 2H), 4.90 (hept, J=6.3 Hz, 1H), 4.18-3.97 (m, 3H), 3.95-3.75 (m, 5H), 2.67 (p, J=5.0 Hz, 2H), 2.36-2.10 (m, 5H), 2.09-1.83 (m, 4H), 1.70-1.50 (m, 3H), 1.50-1.34 (m, 1H), 1.25-1.05 (m, 7H).

### Example 7

### (R)-1-((1R,2R,3S,5R)-3,5-dihydroxy-2-((Z)-7-Isopropoxy-7-oxohept-2-en-1-yl)cyclopentyl)-5-phenylpentan-3-yl (1,3-dihydroxypropan-2-yl) succinate (23)

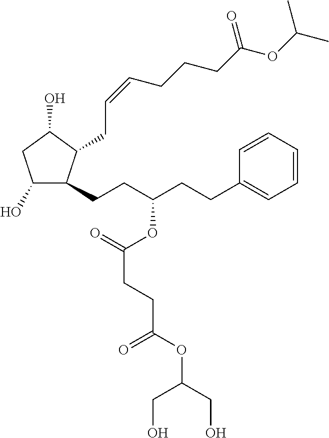 [SMILES: CC(C)OC(=O)CCC/C=C\C[C@@H]1[C@@H](CC[C@H](CCc2ccccc2)OC(=O)CCC(=O)OC(CO)CO)[C@H](O)C[C@@H]1O]

The general procedure for the formation of 15-O-Ester of Prostaglandin (Procedure 4) was followed, using (9) (151.0 mg, 3.03 mmol), 4-nitrophenyl (2-phenyl-1,3-dioxan-5-yl) succinate (163.3 mg, 0.41 mmol) and DMAP (117.1 mg, 0.96 mmol) in anhydrous DCM (10 mL). The residue was dissolved in methanol (10 mL) and stirred for 16 h. The benzylidene ester (22) was obtained. ESI-MS: m/z 717 ([M+Na]⁺).

The general procedure for benzylidene deprotection (Procedure 2) was followed, using (22) (114.2 mg, 0.16 mmol) in 80% acetic acid (5 mL). The mixture was stirred at room temperature for 48 h. The crude material was chromatographed (SiO₂, EtOAc, 100%) to give the title compound (23) as a pale yellow oil. ESI-MS: m/z 629 ([M+Na]*).

### Example 8

### (Z)-isopropyl 7-((1R,2R,3R,5S)-5-hydroxy-3-((3-hydroxy-2-(hydroxymethyl)propanoyl)oxy)-2-((R)-3-hydroxy-5-phenylpentyl)cyclopentyl)hept-5-enoate (25)

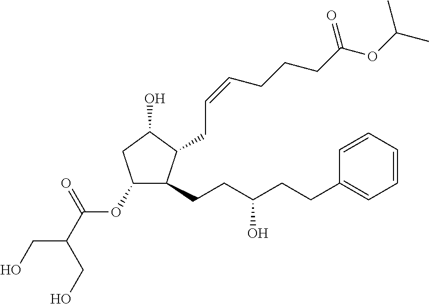 [SMILES: CC(C)OC(=O)CCC/C=C\C[C@@H]1[C@@H](CC[C@@H](O)CCc2ccccc2)[C@H](OC(=O)C(CO)CO)C[C@@H]1O]

A method similar to that described by Gu et al. Org. Lett. 2005, 7(18), 3945 was used. A mixture of PdCl₂ (8.3 mg, 0.03 mmol), LiCl (3.5 mg, 0.08 mmol) in MeOH (1 mL) was heated under reflux until it become a clear solution (about 45 min to 1 h). The MeOH was then removed under reduced pressure, vinyl acetate (2 mL) was added and the solution was concentrated to dryness. The residue was then re-dissolved in vinyl acetate (2 mL) and was added to a mixture of 2-(4-methoxyphenyl)-1,3-dioxane-5-carboxylic acid (270.7 mg, 1.14 mmol) in vinyl acetate (2 mL). The mixture was refluxed for 16 h under nitrogen atmosphere. The solvent was evaporated under reduced pressure and the oily residue was then dissolved in hexane (2 mL). The hexane solution was concentrated and the crude product, vinyl 2-(4-methoxyphenyl)-1,3-dioxane-5-carboxylate was used without further purification. ¹H NMR spectroscopy showed the desired vinyl ester along with some starting material in a ratio of 7:3.

Latanoprost (133.3 mg, 0.31 mmol) and Novozyme 432 (82.3 mg) are dried under vacuum for 3 h. Anhydrous THF (2 mL) and vinyl 2-(4-methoxyphenyl)-1,3-dioxane-5-carboxylate (253.1 mg, 1.08 mmol) are added. The reaction mixture is heated at 64° C. for 16 h. The reaction is quenched with chloroform (2 mL) and filtered. The solvent is removed in vacuo to give the benzylidene ester which is used without further purification.

The general procedure for benzylidene deprotection (Procedure 2) should be followed, using (1R,2R,3R,4S)-4-hydroxy-2-((R)-3-hydroxy-5-phenylpentyl)-3-((Z)-7-isopropoxy-7-oxohept-2-en-1-yl)cyclopentyl 2-phenyl-1,3-dioxane-5-carboxylate in 80% acetic acid. The crude material should be chromatographed (SiO₂, MeOH:CHCl₃, 10%) to give the title compound.

### Example 9

### (1S,2R,3R,4R)-2-((Z)-7-(ethylamino)-7-oxohept-2-en-1-yl)-4-hydroxy-3-((S,E)-3-hydroxy-5-phenylpent-1-en-1-yl)cyclopentyl 3-hydroxy-2-(hydroxymethyl)propanoate (26)

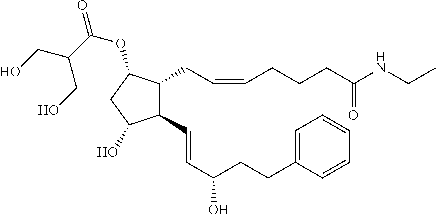 [SMILES: CCNC(=O)CCC/C=C\C[C@@H]1[C@@H](/C=C/[C@@H](O)CCc2ccccc2)[C@H](O)C[C@@H]1OC(=O)C(CO)CO]

To a solution of bimatoprost (800 mg, 1.82 mmol) in dichloromethane (20 ml) was added TBSCl (638 mg, 4.23 mmol), triethylamine (802 μl, 5.76 mmol) and dimethylaminopyridine (40 mg). The solution was stirred at room temperature overnight. DCM (500 ml) was added and the solution was washed with water (3×200 ml). The organic layer was washed with brine, dried over Na₂SO₄, filtered, concentrated in vacuo and purified by flash chromatography (silica, petroleum ether:ethyl acetate 10:1 to 3:1) to give the desired 11,15-TBS-protected product as a colourless oil (650 mg, 52%); ¹H NMR (400 MHz, DMSO) δ 7.71 (t, J=5.0 Hz, 1H), 7.27 (t, J=7.4 Hz, 2H), 7.20-7.08 (m, 3H), 5.50 (dd, J=15.4, 5.3 Hz, 1H), 5.46-5.34 (m, 2H), 5.34-5.19 (m, 1H), 4.47 (d, J=4.8 Hz, 1H), 4.17 (dd, J=5.7 Hz, 1H), 3.99-3.88 (m, 1H), 3.84 (dd, J=13.9, 8.0 Hz, 1H), 3.12-2.93 (m, 2H), 2.59 (dd, J=9.7, 6.0 Hz, 2H), 2.38-2.18 (m, 2H), 2.17-2.03 (m, 1H), 1.96 (dt, J=19.1, 7.4 Hz, 5H), 1.74 (dd, J=9.9, 5.2 Hz, 2H), 1.48 (dt, J=15.0, 7.4 Hz, 2H), 1.42 (dd, J=5.7, 1.8 Hz, 1H), 1.37-1.17 (m, 1H), 0.98 (t, J=7.2 Hz, 3H), 0.88 (s, 9H), 0.82 (s, 9H), 0.04 (s, 3H), 0.01 (s, 3H), −0.00 (s, 3H), −0.02 (s, 3H).

To a solution of the 11,15-TBS-protected product (430 mg, 0.67 mmol) and 2-phenyl-1,3-dioxane-5-carboxylic acid (180 mg, 0.87 mmol) in DMF (3 ml) were added HATU (509 mg, 1.34 mmol) and DMAP (30 mg). The reaction vessel was sealed and heated in a microwave at 140 C for 20 min. The reaction was allowed to cool to room temperature and the residue was purified by flash chromatography (silica, petroleum spirit:ethyl acetate, 3:1) to give the desired benzylidene ester as a colourless oil (190 mg, 34.1%). ¹H NMR (400 MHz, DMSO) δ 7.75 (t, J=5.2 Hz, 1H), 7.52-7.35 (m, 5H), 7.31 (t, J=7.4 Hz, 2H), 7.19 (t, J=8.5 Hz, 3H), 5.64 (dd, J=15.3, 5.5 Hz, 1H), 5.54 (s, 1H), 5.52 (dd, J=23.3, 16.8 Hz, 1H), 5.42-5.28 (m, 2H), 5.01 (t, J=4.5 Hz, 1H), 4.43-4.33 (m, 2H), 4.23 (dd, J=11.5, 5.9 Hz, 1H), 4.06-4.01 (m, 1H), 3.98 (dd, J=11.4, 3.9 Hz, 2H), 3.17-3.01 (m, 3H), 2.63 (dd, J=9.6, 6.6 Hz, 2H), 2.44 (ddd, J=14.3, 8.2, 5.7 Hz, 1H), 2.39-2.29 (m, 1H), 2.09 (t, J=7.5 Hz, 2H), 2.03 (t, J=7.5 Hz, 2H), 2.00-1.88 (m, 2H), 1.85-1.73 (m, 2H), 1.73-1.63 (m, 1H), 1.52 (dt, J=11.8, 6.1 Hz, 2H), 1.46 (d, J=4.6 Hz, 1H), 1.00 (t, J=7.2 Hz, 3H), 0.92 (s, 9H), 0.86 (s, 9H), 0.08 (s, 3H), 0.05 (s, 3H), 0.04 (s, 3H), 0.03 (s, 3H).

To a solution of the above product (180 mg, 0.22 mmol) in THF (0.5 ml) was added TBAF (1.0 M in THF, 0.65 ml, 0.65 mmol). The solution was stirred at room temperature overnight. The reaction mixture was concentrated in vacuo and the residue taken up in ethyl acetate (200 ml) and washed with water (3×200 ml). The organic layer was washed with brine, dried over Na₂SO₄, filtered, concentrated in vacuo and purified by flash chromatography (silica, DCM:MeOH, 50:1 to 20:1) to give an oil (70 mg). TLC (petroleum:ethyl acetate, 3:1) and ¹H NMR spectroscopy showed mono TBS-protected material so it was subjected to a repeat of the above conditions and purified to give 40 mg of a mixture of the desired material and mono TBS-protected material which was taken on without further purification.

The general procedure for benzylidene deprotection (Procedure 2) was then followed, using the product above (32.1 mg, 0.05 mmol) in 80% acetic acid (2 mL) stirred at room temperature for 48 h. The crude material was chromatographed (SiO₂, MeOH:CHCl₃, 10%) to give the title compound (22.4 mg) as a pale yellow oil. ESI-MS: m/z 563 ([M+2Na]⁺).

### Synthesis of Polymer Drua Conjugates

### Example 10

### Polyurethane of (Z)-1,3-dihydroxypropan-2-yl 7-((1R,2R,3R,5S)-3,5-dihydroxy-2-((R)-3-hydroxy-5-phenylpentyl)cyclopentyl)hept-5-enoate and ELDI

The general procedure for polymerisation, Method A, was followed, using (5) (108.2 mg, 0.23 mmol), ethyl ester of lysine diisocyanate (ELDI) (68.4 mg, 0.30 mmol) and DBTDL (11.0 mg, 0.02 mmol) in anhydrous THF (1 mL). The title polymer drug conjugate (87.5 mg) was obtained as a white solid. (GPC in DMF showed Mw=2.583 kDa with polydispersity (PDI)=1.25).

The polymer was then melt extruded into rods of 1.0 mm diameter at melt temperature of 40° C. and @5 mL/min using a micro extruder.

### Example 11

### Polyurethane of (Z)-1,3-dihydroxypropan-2-yl 7-((1R,2R,3R,5S)-3,5-dihydroxy-2-((R)-3-hydroxy-5-phenylpentyl)cyclopentyl)hept-5-enoate and HDI

The general procedure for Polymerisation Method B, was followed, using (5) (70.2 mg, 0.15 mmol), hexamethylene diisocyanate (HDI) (32.9 mg, 0.20 mmol) and DBTDL (12.0 mg, 0.02 mmol) in anhydrous THF (1 mL) at 45° C. The title polymer drug conjugate (38.8 mg) was obtained as a white solid. (GPC in DMF showed Mw=143 kDa with PDI=3.12).

The polymer was then melt extruded into rods of 0.3 mm diameter at melt temperature of 75° C. and @5 mL/min using a micro extruder.

### Example 12

### Polyurethane of (Z)-1,3-dihydroxypropan-2-yl 7-((1R,2R,3R,5S)-3,5-dihydroxy-2-((R)-3-hydroxy-5-phenylpentyl)cyclopentyl)hept-5-enoate and DVDIP

The general procedure for Polymerisation Method A, was followed, using (5) (102.1 mg, 0.22 mmol), propane-1,3-diyl bis(2-isocyanato-3-methylbutanoate) (DVDIP) (95.2 mg, 0.29 mmol) and DBTDL (11.0 mg, 0.02 mmol) in anhydrous THF (1 mL). The title polymer drug conjugate (93.3 mg) was obtained as a white solid. (GPC in DMF showed Mw=2.325 kDa with PDI=1.095).

The polymer was then melt extruded into rods of 1.0 mm diameter at melt temperature of 40° C. and @5 mL/min using a micro extruder.

### Example 13

### Polyurethane of (Z)-1,3-dihydroxypropan-2-yl 7-((1R,2R,3R,5S)-3,5-dihydroxy-2-((R)-3-hydroxy-5-phenylpentyl)cyclopentyl)hept-5-enoate, ELDI and PEG (1000)

The general procedure for Polymerisation Method C, was followed, using (5) (57.5 mg, 0.12 mmol), ELDI (55.9 mg, 0.25 mmol), PEG (1000) (140.5 mg, 0.15 mmol) and DBTDL (12.8 mg, 0.02 mmol) in anhydrous THF (1 mL) at 45° C. The title polymer drug conjugate was obtained as a white cloudy oil. (GPC in DMF showed Mw=23.5 kDa with PDI=1.14)

### Example 14

### Polyurethane of (Z)-1,3-dihydroxypropan-2-yl 7-((1R,2R,3R,5S)-3,5-dihydroxy-2-((R)-3-hydroxy-5-phenylpentyl)cyclopentyl)hept-5-enoate, ELDI and PCL (1000)

The general procedure for Polymerisation Method C, was followed, using (5) (54.5 mg, 0.12 mmol), ELDI (54.8 mg, 0.24 mmol), PCL (1000) (118.1 mg, 0.12 mmol) and DBTDL (13.0 mg, 0.02 mmol) in anhydrous THF (1 mL) at 45° C. The title polymer drug conjugate was obtained as a white cloudy oil. (GPC in DMF showed Mw=22.9 kDa with PDI=1.30)

### Example 15

### Poly(urethane-ester) of (Z)-1,3-dihydroxypropan-2-yl 7-((1R,2R,3R,5S)-3,5-dihydroxy-2-((R)-3-hydroxy-5-phenylpentyl)cyclopentyl)hept-5-enoate, ELDI and PLGA

The general procedure for Polymerisation Method C, was followed, using (5) (54.6 mg, 0.12 mmol), ELDI (62.1 mg, 0.27 mmol), PLGA (50:50) (Mw=1175) (138.3 mg, 0.12 mmol) and DBTDL (9.9 mg, 0.02 mmol) in anhydrous THF (1 mL) at 45° C. The title polymer drug conjugate was obtained as a solid. (GPC in DMF showed Mw=11.9 kDa with PDI=2.77)

### Example 16

### Polyurethane of (Z)-3-hydroxy-2-(hydroxymethyl)-2-methylpropyl 7-((1R,2R,3R,5S)-3,5-dihydroxy-2-((R)-3-hydroxy-5-phenylpentyl)cyclopentyl)hept-5-enoate and DVDIP

The general procedure for Polymerisation Method A, was followed, using (2) (89.8 mg, 0.18 mmol), propane-1,3-diyl bis(2-isocyanato-3-methylbutanoate) (70.4 mg, 0.22 mmol) and DBTDL (12.0 mg, 0.02 mmol) in anhydrous THF (1 mL). The title polymer drug conjugate (51.7 mg) was obtained as a white solid. (GPC in DMF showed Mw=6.093 kDa with PDI=1.34).

The polymer was then melt extruded into rods of 1.0 mm diameter at melt temperature of 40° C. and @5 mL/min using a micro extruder.

### Example 17

### Polyurethane of (Z)-3-hydroxy-2-(hydroxymethyl)-2-methylpropyl 7-((1R,2R,3R,5S)-3,5-dihydroxy-2-((R)-3-hydroxy-5-phenylpentyl)cyclopentyl)hept-5-enoate and DVDIP

The general procedure for Polymerisation Method A, was followed, using (2) (44 mol %), propane-1,3-diyl bis(2-isocyanato-3-methylbutanoate) (56 mol %) and DBTDL (catalytic) in anhydrous THF (1 mL). The title polymer drug conjugate was obtained as a white solid.

### Example 18

### Polyurethane of (Z)-3-hydroxy-2-(hydroxymethyl)-2-methylpropyl 7-((1R,2R,3R,5S)-3,5-dihydroxy-2-((R)-3-hydroxy-5-phenylpentyl)cyclopentyl)hept-5-enoate and DVDIP

The general procedure for Polymerisation Method B, was followed, using (2) (47 mol %), propane-1,3-diyl bis(2-isocyanato-3-methylbutanoate) (53 mol %) and DBTDL (catalytic) in anhydrous THF (1 mL). The title polymer drug conjugate was obtained as a white solid.

### Example 19

### Polyurethane of (Z)-3-hydroxy-2-(hydroxymethyl)-2-methylpropyl 7-((1R,2R,3R,5S)-3,5-dihydroxy-2-((R,E)-3-hydroxy-4-(3-(trifluoromethyl)phenoxy)but-1-en-1-yl)cyclopentyl)hept-5-enoate and ELDI

The general procedure for Polymerisation Method B, was followed, using (24) (38.7 mg, 0.069 mmol), ELDI (18.6 mg, 0.082 mmol) and DBTDL (9.3 mg, 0.015 mmol) in anhydrous THF (1 mL) at 45° C. The title polymer drug conjugate was obtained as a cream foam (28.3 mg).

### Example 20

### Polyurethane of 1,3-dihydroxypropan-2-yl 4-(((Z)-7-((1R,2R,3R,5S)-3,5-dihydroxy-2-((R)-3-hydroxy-5-phenylpentyl)cyclopentyl)hept-5-enoyl)oxy)benzoate and ELDI

The general procedure for Polymerisation Method B, was followed, using (6) (111.3 mg, 0.19 mmol), ELDI (56.6 mg, 0.25 mmol) and DBTDL (11.4 mg, 0.02 mmol) in anhydrous THF (1 mL) at 45° C. The title polymer drug conjugate (128.2 mg) was obtained as a white solid. (GPC in DMF showed Mw=31.8 kDa with PDI=4.35).

The polymer was then melt extruded into rods of 0.6 mm diameter at melt temperature of 85° C. and @5 mL/min using a micro extruder. GPC in DMF showed Mw=34.4 kDa with PDI=2.96.

### Example 21

### Polyurethane of (Z)-isopropyl 7-((1R,2R,3R,5S)-3,5-dihydroxy-2-((R)-3-((3-hydroxy-2-(hydroxymethyl)propanoyl)oxy)-5-phenylpentyl)cyclopentyl)hept-5-enoate and ELDI

The general procedure for Polymerisation Method B, was followed, using (14) (81.1 mg, 0.15 mmol), ELDI (39.4 mg, 0.18 mmol) and DBTDL (11.0 mg, 0.02 mmol) in anhydrous THF (1 mL) at 45° C. The title polymer drug conjugate (10 mg) was obtained as a clear colourless semi-solid.

### Example 22

### Polyurethane of (2)-isopropyl 7-((1R,2R,3R,5S)-3,5-dihydroxy-2-((R,E)-3-((3-hydroxy-2-(hydroxymethyl)propanoyl)oxy)-4-(3-(trifluoromethyl)phenoxy)but-1-en-1-yl)cyclopentyl)hept-5-enoate and ELDI

The general procedure for polymerisation, Method B, was followed, using (15) (34.7 mg, 0.06 mmol), ELDI (15.0 mg, 0.07 mmol) and DBTDL (11.4 mg, 0.02 mmol) in anhydrous THF (1 mL) at 45° C. The title polymer drug conjugate (36.5 mg) was obtained as a white solid. (GPC in DMF showed Mw=19.9 kDa with PDI=2.50)

The polymer was then melt extruded into rods of 0.3 mm diameter at melt temperature of 75° C. and @5 mL/min using a micro extruder.

### Example 23

### Polyurethane of (1S,2R,3R,4R)-2-((Z)-7-(ethylamino)-7-oxohept-2-en-1-yl)-4-hydroxy-3-((S,E)-3-hydroxy-5-phenylpent-1-en-1-yl)cyclopentyl 3-hydroxy-2-(hydroxymethyl)propanoate and ELDI

The general procedure for Polymerisation Method B, was followed, using (1S,2R,3R,4R)-2-((Z)-7-(ethylamino)-7-oxohept-2-en-1-yl)-4-hydroxy-3-((S,E)-3-hydroxy-5-phenylpent-1-en-1-yl)cyclopentyl 3-hydroxy-2-(hydroxymethyl)propanoate (26) (22.4 mg, 0.043 mmol), ELDI (13.6 mg, 0.060 mmol) and DBTDL (11.0 mg, 0.017 mmol) in anhydrous THF (1 mL) at 45° C.

The title polymer drug conjugate was obtained as a white solid (30.1 mg).

### Example 24

### Polyurethane of (Z)-3-hydroxy-2-(hydroxymethyl)-2-methylpropyl 7-((1R,2R,3R,5S)-3,5-dihydroxy-2-((R)-3-hydroxy-5-phenylpentyl)cyclopentyl)hept-5-enoate and ELDI

The general procedure for Polymerisation Method B, was followed, using (2) (16.2 mg, 0.033 mmol), ELDI (15.6 mg, 0.07 mmol) and DBTDL (10.4 mg, 0.016 mmol) in anhydrous THF (1 mL) at 45° C. The title polymer drug conjugate (18.4 mg) was obtained as a white solid.

### Example 25

### Polyurethane of (R)-1-((1R,2R,3S,5R)-3,5-dihydroxy-2-((Z)-7-Isopropoxy-7-oxohept-2-en-1-yl)cyclopentyl)-5-phenylpentan-3-yl (1,3-dihydroxypropan-2-yl) succinate and ELDI

The general procedure for Polymerisation Method B was followed, using (23) (236.9 mg, 0.39 mmol), ELDI (103.2 mg, 0.456 mmol) and DBTDL (10.4 mg, 0.016 mmol) in anhydrous THF (1 mL) at 45° C. The title polymer drug conjugate was obtained as a cream solid (81 mg).

The above polymer drug conjugates are summarised in Table 2.

TABLE 2
Prostaglandin Polymer Drug Conjugate Examples:

| | | Isocyanate | Hydrophilic | | |
|---|---|---|---|---|---|
| | Drug-Monomer Conjugate | Monomer | Component | | Polym. |
| No. | (mol %) | (mol %) | (mol %) | Linkage | Method |
| 10 | Latanoprost-2-MG (5) | ELDI | — | 1-COOH | A |
| | (43%) | (57%) | | | |
| 11 | Latanoprost-2-MG (5) | HDI | — | 1-COOH | B |
| | (47%) | (53%) | | | |
| 12 | Latanoprost-2-MG (5) | DVDIP | — | 1-COOH | A |
| | (43%) | (57%) | | | |
| 13 | Latanoprost-2-MG (5) | ELDI | PEG1000 | 1-COOH | C |
| | (25%) | (50%) | (25%) | | |
| 14 | Latanoprost-2-MG (5) | ELDI | PCL | 1-COOH | C |
| | (25%) | (50%) | (25%) | | |
| 15 | Latanoprost-2-MG (5) | ELDI | PLGA | 1-COOH | C |
| | (25%) | (50%) | (25%) | | |
| 16 | Latanoprost-THE (2) | DVDIP | — | 1-COOH | A |
| | (45%) | (55%) | | | |
| 17 | Latanoprost-THE (2) | DVDIP | — | 1-COOH | A |
| | (44%) | (56%) | | | |
| 18 | Latanoprost-THE (2) | DVDIP | — | 1-COOH | B |
| | (47%) | (53%) | | | |
| 19 | Travoprost-THE (24) | ELDI | — | 1-COOH | B |
| | (47%) | (53%) | | | |
| 20 | Latanoprost-p-hydroxybenzoic | ELDI | — | 1-COOH | B |
| | acid-2-MG (6) | (57%) | | | |
| | (43%) | | | | |
| 21 | Latanoprost- | ELDI | — | 15-OH | B |
| | dihydroxyisobutyric acid (14) | (55%) | | | |
| | (45%) | | | | |
| 22 | Travoprost-dihydroxyisobutyric | ELDI | — | 15-OH | B |
| | acid (15) | (53%) | | | |
| | (47%) | | | | |
| 23 | Bimatoprost- | ELDI | — | 9-OH | B |
| | dihydroxyisobutyric acid (26) | (53%) | | | |
| | (47%) | | | | |
| 24 | Latanoprost-THE (2) | ELDI | — | 1-COOH | B |
| | (32%) | (68%) | | | |
| 25 | Latanoprost-succinate-2-MG | ELDI | — | 1-COOH | B |
| | (23) | (54%) | | | |
| | (46%) | | | | |

### Drug Delivery System

Drug delivery systems comprising a polymer-drug conjugate of the invention admixed with a hydrophilic polymer were also prepared.

### Example 26

### Polyurethane of (Z)-1,3-dihydroxypropan-2-yl 7-((1R,2R,3R,5S)-3,5-dihydroxy-2-((R)-3-hydroxy-5-phenylpentyl)cyclopentyl)hept-5-enoate and ELDI blended with PEG (3000)

Following polymerisation method D, the polyurethane of (Z)-1,3-dihydroxypropan-2-yl 7-((1R,2R,3R,5S)-3,5-dihydroxy-2-((R)-3-hydroxy-5-phenylpentyl)cyclopentyl)hept-5-enoate and ELDI (Example 10) (52.3 mg) and PEG (3000) (55.5 mg) were dissolved in anhydrous DCM (1 mL) and stirred at room temperature for 1 h. Solvent was removed in vacuo to give the blend material as an off-white semi solid.

### Example 27

### Polyurethane of (Z)-1,3-dihydroxypropan-2-yl 7-((1R,2R,3R,5S)-3,5-dihydroxy-2-((R)-3-hydroxy-5-phenylpentyl)cyclopentyl)hept-5-enoate and DVDIP blended with PEG (3000)

Following polymerisation method D, the polyurethane of (Z)-1,3-dihydroxypropan-2-yl 7-((1R,2R,3R,5S)-3,5-dihydroxy-2-((R)-3-hydroxy-5-phenylpentyl)cyclopentyl)hept-5-enoate and DVDIP (Example 12) (63.9 mg) and PEG (3000) (64.2 mg) were dissolved in anhydrous DCM (1 mL) and stirred at room temperature for 1 h. Solvent was removed in vacuo to give the blend material as an off-white semi solid.

The above drug delivery systems are summarised in Table 3.

TABLE 3
Drug Delivery System Examples:

| | | Isocyanate | Hydrophilic | | |
|---|---|---|---|---|---|
| | Drug-Monomer Conjugate | Monomer | Component | | |
| No. | (mol %) | (mol %) | (mol %) | Linkage | Polym. Method |
| 26 | Latanoprost-2-MG (5) | ELDI | PEG3000 | 1-COOH | D |
| | | | (50%) | | |
| 27 | Latanoprost-2-MG (5) | DVDIP | PEG3000 | 1-COOH | D |
| | | | (51%) | | |

### General Melt Extrusion Method

The polymer drug conjugates can be formed into a rod-shaped fibre or implant by a simple melt extrusion method. The polymer drug conjugate is forced under pressure and at elevated temperatures through a die to provide a continuous feed of rod-shaped material with a fixed outer diameter. The rod-shaped material may then be cut with a hot knife in predetermined lengths to provide the final product.

A basic plunger based extruder is used to produce the final product. Firstly, a barrel is charged with the material to be extruded. At one end of the barrel is a die with a single cylindrical shaped hole (ranging in diameter form 0.3-2.0 mm) from which the material extrudes. At the other end of the barrel is a plunger that forces the contents of the barrel through the die at a constant rate. The barrel and die are heated, up to 300° C. if necessary, though more usually 40-120° C., to ensure the material within the barrel is extruded at or close to its melting point.

The exudate from the die is air cooled prior to handling and may be dried in a vacuum oven if deemed necessary.

A number of the polymers were melt extruded into rods of various diameter. The melt temperature varied from 40 to 120° C. and the extrusion was conducted 5 mL/min using a micro extruder.

TABLE 4
Table of rod-shaped fibres and implants produced (conducted @ 5
mL/min using a micro extruder) with various polymer drug conjugates.

| Polymer Drug | | |
|---|---|---|
| Conjugate Example | Extrusion | Rod Diameter |
| No. | Temperature (° C.) | (mm) |
| 10 | 40 | 1.0 |
| 12 | 40 | 1.0 |
| 16 | 40 | 1.0 |
| 11 | 75 | 0.3 |
| 17 | 120 | 1.0 |
| 18 | 80 | 0.6 |
| 20 | 85 | 0.6 |
| 22 | 75 | 0.3 |

### Drug Release Method

Following in vitro release guidelines recommended by the International Organisation of Standardisation, rod-shaped samples prepared by melt extrusion, were suspended in wire baskets which were immersed in isotonic phosphate buffer (IPB), adjusted to pH 7.4 using orthophosphoric acid and containing 0.01% sodium azide as a preservative, and incubated at 37° C. with continuous stirring. Aliquots of the receptor solution were removed for analysis at predetermined time points until drug release from the polymer no longer increased.

The amount of prostaglandin drug released from the rods at the various time points was quantified by reverse phase high performance liquid chromatography (HPLC) with a UV absorbance detector and analyte separation was performed on a C18 column either isocratically or with a gradient system using a degassed mobile phase.

Using the above method, the rate of release of the prostaglandin drug latanoprost from various polymer-drug conjugates was determined. The results are shown below in Table 5.

TABLE 5
Release rate of latanoprost free acid
from latanoprost-polymer conjugates.

| | Release Rate (ng of free | |
|---|---|---|
| | acid Latanoprost/mg | Period of zero-order |
| Polymer Drug | polymer drug conjugate/ | release |
| Conjugate Example | 24 hours) | (days) |
| 10 | 899 | 61 |
| 12 | 637 | 61 |
| 16 | 138 | 61 |
| 26 | 540 | 60 |
| 27 | 465 | 60 |

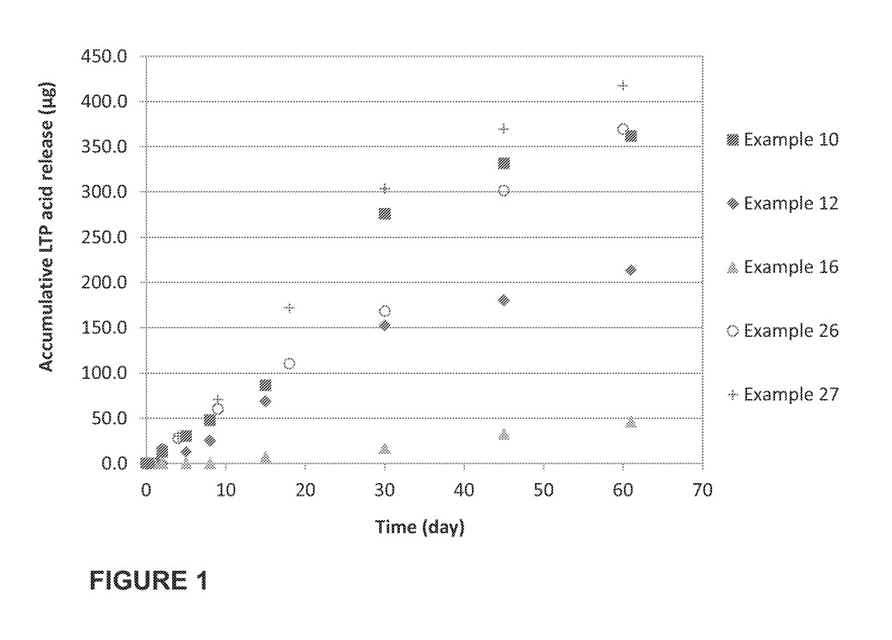

The rate of release from the polymer drug conjugates was measured over 60 days and zero-order drug release was exhibited over the entire time (see FIG. 1). The zero order release profile indicates that a constant amount of prostaglandin drug is released per time period, providing a more constant dose of drug to the site of delivery.

It is anticipated the other polymer-drug conjugates of the invention will behave similarly, exhibiting zero-order release of the prostaglandin drug over time, typically at least 60 days.

### Ocular Implant Production

The polymer-drug conjugate or material containing the polymer-drug conjugate can be formed into a device suitably shaped to facilitate delivery to the eye. One such device is a rod-shaped implant able to be housed within the lumen of a 20 to 23 gauge needle. The outer diameter of the implant would be about 0.4 mm. The length of the implant can be selected to deliver the required dose of prostaglandin drug, Typical size of an implant is 0.3 mm diameter×1-2 mm in length. The implant can be administered subconjunctivally to the affected eye where it would absorb moisture from surrounding tissue to trigger release of the prostaglandin drug and polymer erosion.

One method that could be used to produce the rod-shaped implant would involve melt-extrusion, where the polymer-drug conjugate or material containing the polymer drug conjugate is forced under pressure and at elevated temperatures through a die to provide a continuous feed of rod-shaped material with an outer diameter of about 0.4 mm. The rod-shaped material may then be cut with a hot knife at predefined intervals to provide the final implant.

In one example a basic plunger based extruder is used to produce the implant. Firstly, a barrel is charged with the material to be extruded. At one end of the barrel is a die with a single cylindrical shaped hole about 0.4 mm in diameter from which the material extrudes. At the other end of the barrel is a plunger that forces the contents of the barrel through the die at a constant rate. The barrel and die are heated to ensure the material within the barrel and extruded are at or close to their melting point (typically greater than 70° C.).

In another example a single screw extruder is used to produce the implant. The material to be extruded enters through a feed throat (an opening near the rear of the barrel) and comes into contact with the screw. The rotating screw (normally turning at up to 120 rpm) forces the material forward into the barrel which is heated to the desired melt temperature of the molten plastic (typically greater than 70° C.). Typically, heating zones gradually increase the temperature of the barrel from the rear (where the plastic enters) to the front (where the die is located). This allows the material to melt gradually as it is pushed through the barrel and lowers the risk of overheating which may cause degradation in the polymer. The high pressure and friction of the material inside the barrel also contributes heat to the process. Also the extruder can be operated in a constant flow rate mode with the pressure varied to maintain flow of material or constant pressure mode with the rate of screw rotation varied to maintain a constant pressure. After passing through the barrel the molten material enters the die, which gives the final product its profile.

The exudate from the die of either of these two methods is cooled and this is usually achieved by pulling the exudate through a water bath or a cooling curtain of air.

Those skilled in the art will appreciate that the invention described herein is susceptible to variations and modifications other than those specifically described. It is understood that the invention includes all such variations and modifications which fall within the spirit and scope of the present invention.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

1.-32. (canceled)

33. A polymer-drug conjugate comprising a polymer backbone and a prostaglandin drug conjugated to the polymer backbone via an ester, anhydride or carbonate linking group.

34. A polymer-drug conjugate according to claim 33 comprising a polymer backbone and a PGF_{2α} class of prostaglandin or substituted prostaglandin conjugated to the polymer backbone via an ester, anhydride or carbonate linking group.

35. A polymer-drug conjugate according to claim 33 wherein the ester linking group links the prostaglandin drug at a position selected from the 1, 9, 11 and 15 position.

36. A polymer-drug conjugate according to claim 33, wherein the polymer-drug conjugate comprises a plurality of prostaglandin drugs of formula (XX): 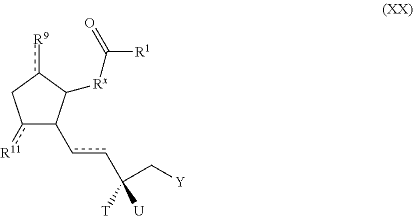
wherein:
R^{x} is a straight chain aliphatic of six carbon atoms optionally comprising one or two substituents selected from the group consisting of oxo (═O) and hydroxy; [symbol]
represents a double or single bond;
T and U are selected from the group consisting of where T and U together form oxo (═O), where T and U are each halo, and where T is R¹⁵ and U is hydrogen;
Y is optionally substituted C₄ to C₁₀ hydrocarbyl or optionally substituted C₄ to C₁₀ hydrocarbyloxy; and
one of R¹, R⁹, R¹¹ and R¹⁵ is linked to the polymer backbone and wherein:
R⁹, R¹¹ and R¹⁵ when linked to the polymer backbone are the alcohol residue of an ester or carbonate linking group and R¹ when linked to the polymer backbone forms the acid residue of an ester or anhydride linking group; and
R¹ when not linked to the backbone is selected from the group consisting of —OH, —O(C₁₋₆ alkyl), and —NR^{a}R^{b} where R^{a} and R^{b} are each independently selected from the group consisting of H and C₁, alkyl;
R⁹ and R¹¹ when not linked to the polymer backbone are both hydroxy or one is hydroxy and one is oxo and where one of R⁹ and R¹¹ is linked to the backbone, the other is hydroxy or oxo; and
when R¹⁵ is not linked to the backbone then T is hydroxy and U is hydrogen, or T and U are each fluoro, or T and U together form oxo.

37. A polymer-drug conjugate according to claim 36, wherein the polymer-drug conjugate comprises a plurality of prostaglandin drugs of formula (XXi): 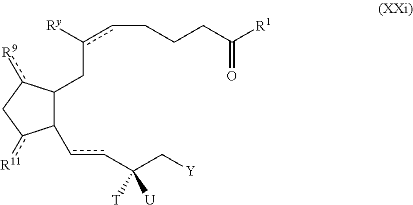
where: [symbol]
represents a double or single bond;
T and U are selected from the group consisting of where T and U together form oxo (═O), where T and U are each halo, and where T is R¹⁵ and U is hydrogen;
R^{y} is an optional substituent selected from the group consisting of oxo and hydroxy;
Y is optionally substituted C₄ to C₁₀ hydrocarbyl or optionally substituted C₄ to C₁₀ hydrocarbyloxy; and
one of R¹, R⁹, R¹¹ and R¹⁵ is linked to the polymer backbone and wherein:
R⁹, R¹¹ and R¹⁵ when linked to the polymer backbone are the alcohol residue of an ester or carbonate linking group and R¹ when linked to the polymer backbone forms the acid residue of an ester or anhydride linking group; and
R¹ when not linked to the backbone is selected from the group consisting of OH, —O(C₁₋₆ alkyl), and —NR^{a}R^{b} where R^{a} and R^{b} are each independently selected from the group consisting of H and C₁₋₆ alkyl;
R⁹ and R¹¹ when not linked to the polymer backbone are both hydroxy or one is hydroxy and one is oxo and where one of R⁹ and R¹¹ is linked to the backbone, the other is hydroxy or oxo; and
when R¹⁵ is not linked to the backbone then T is hydroxy and U is hydrogen, or T and U are each fluoro, or T and U together form oxo.

38. A polymer-drug conjugate according to claim 33 wherein the polymer-drug conjugate comprising as part of its polymer backbone a moiety of general formula (I): 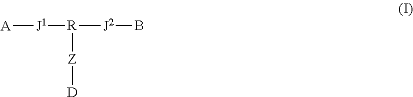
where:
A and B, which may be the same or different, represent the remainder of the polymer backbone and are (i) attached to the -J¹-R(ZD)-J²- moiety as shown in formula (I) via a bioerodible moiety, and (ii) each formed from monomeric units that are coupled via bioerodible moieties;
J¹ and J² are independently selected from the group consisting of oxygen, C(O), and NR^{a} where R^{a} is hydrogen or C₁ to C₆ alkyl;
R is an optionally substituted hydrocarbon;
Z is a linking group;
D is a prostaglandin drug of formula (XX); and
D and Z together form an ester, anhydride or carbonate linking group.

39. A polymer drug conjugate according to claim 38 wherein:
(a) the group D is a prostaglandin drug of formula (XX), wherein R¹ is the acid residue of an ester or anhydride linking group and Z is of a formula selected from the group consisting of:
(i) (R) —O— (D);
(ii) (R) -Q-Ar—O— (D);
(iii) (R) -Q-C₁-C₁₂alkylene-O— (D);
(iv) (R) -Q-Ar-Q-C₁-C₁₂alkylene-O— (D);
(v) (R) -Q-C₁-C₁₂alkylene-Q-Ar—O(D);
(vi) (R) -Q-C₁-C₁₂alkylene-Q-Ar-Q-C₁-C₁₂alkylene-O— (D);
(vii) (R) —OC(O)— (D);
(Viii) (R) -Q-Ar—OC(O)— (D); and
(ix) (R) -Q-C₁-C₁₂alkylene-OC(O)— (D).
(b) the group D is the prostaglandin drug of formula (XX) wherein one of R⁹, R¹¹ and R¹⁵ is the hydroxy residue (—O—) of an ester or carbonate linking group and Z is of formula selected from the group consisting of
(i) (R) —C(O) (D);
(ii) (R) —OC(O)— (D);
(ii) (R) -Q-Ar—C(O)— (D);
(iii) (R) -Q-C₁-C₁₂alkylene-C(O)— (D);
(iv) (R) -Q-Ar-Q-C₁-C₁₂alkylene-C(O)— (D);
(v) (R) -Q-Ar-Q-C₁-C₂alkylene-OC(O)— (D);
(vi) (R) -Q-C₁-C₁₂alkylene-Q-Ar—C(O) (D); and
(vii) (R) -Q-C₁-C₁₂alkylene-Q-Ar-Q-C₁-C₁₂alkylene-C(O)— (D);
wherein:
(R) indicates the end of the linking group bonded to the R group and (D) indicates the end of the linking group bonded to the prostaglandin drug D;
Ar is optionally substituted aromatic or heteroaromatic hydrocarbon; and
Q is selected from the group consisting of —O—, —C(O)—, —O—C(O)—, —C(O)—O—, —C(O)OC(O)—, —C(O)NR^{a}C(O)—, —OC(O)NR^{a}—, —NR^{a}C(O)O—, —NR^{a}—, —NR^{a}C(O)NR^{a}—, —NR^{a}C(O)—, —C(O)NR^{a}—, —S—, —O—C(S)—, —C(S)—O—, —S—C(O)—, —C(O)—S—, —NR^{a}C(S)—, and —C(S)NR^{a}—, where R^{a} is hydrogen or C₁ to C₆ alkyl.

40. A polymer-drug conjugate according to claim 36 wherein the prostaglandin drug (D) is of formula: 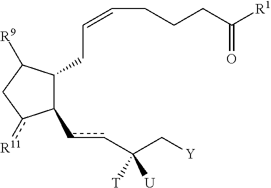
wherein R¹, R⁹, R¹¹, T, U and Y are as herein defined.

41. A polymer-drug conjugate according to claim 36 wherein the prostaglandin drug (D) is selected from the group consisting of: 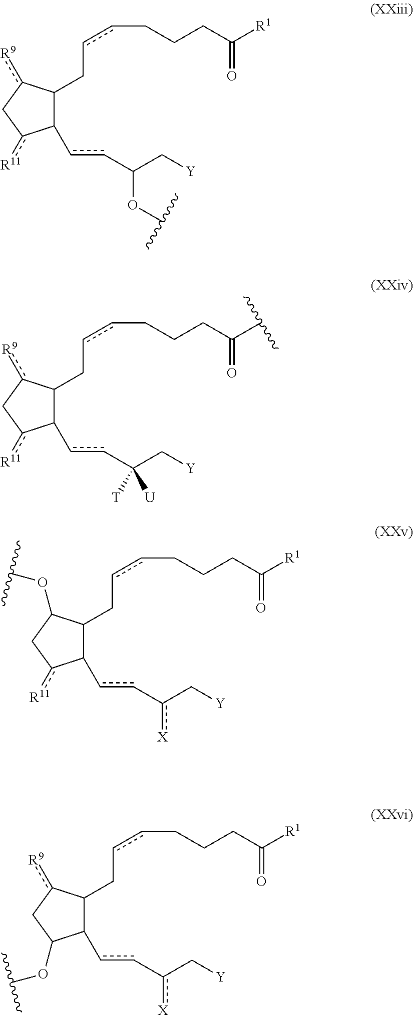
wherein: [symbol]
represents the point of attachment of the prostaglandin drug to linking group Z; [symbol]
represents a double or single bond;
Y is optionally substituted C₄ to C₁₀ hydrocarbyl or optionally substituted C₄ to C₁₀ hydrocarbyloxy;
in formulae (XXiii), (XXv) and (XXvi) R¹ is hydroxy, C₁ to C₆ alkoxy or C₁ to C₆ alkylamino (preferably, isopropoxy or ethylamino);
in formulae (XXiii) and (XXiv) R⁹ and R¹″ are hydroxy or one of R⁹ and R¹¹ is oxo and the other is hydroxy;
in formula (XXv) R¹¹ is hydroxy or oxo and X is O or hydroxy;
in formula (XXvi) R⁹ is hydroxy or oxo; and
in formulae (XXiv) and (XXvi) T is hydroxy and U is hydrogen, or T and U are both fluoro, or T and U together form oxo.

42. A polymer-drug conjugate according to claim 33 wherein the polymer backbone is a polyurethane, polyester, polyether, or a combination thereof, or a copolymer thereof.

43. A polymer-drug conjugate according to claim 33 comprising as part of its polymer backbone a moiety of general formula (Ic): 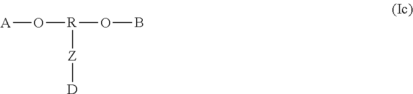
where:
A and B, which may be the same or different, represent the remainder of the polymer backbone and are (i) attached to the —O—R(ZD)-O— moiety as shown in formula (I) via a bioerodible moiety, and (ii) each formed from monomeric units that are coupled via bioerodible moieties;
R is an optionally substituted hydrocarbon;
Z is a linking group; and
D is a releasable drug selected from prostaglandin drugs of general formulae (II) and (III): 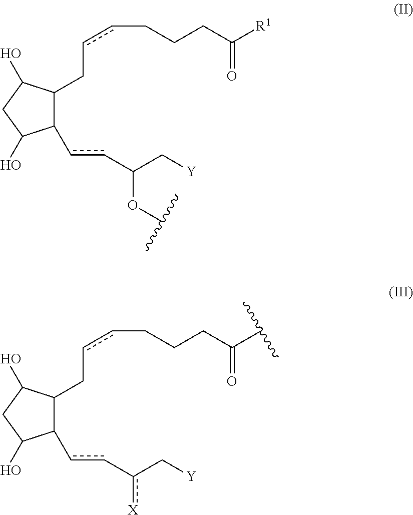
where [symbol] represents a double bond or single bond, [symbol] represents where the prostaglandin analogue is attached to the linking group Z, R¹ is hydroxy, C₁₋₆alkoxy or C₁₋₆alkylamino, X is O, OH or difluoro, and Y is selected from —(CH₂)₃CH₃, —OC₆H₄(meta-CF₃), (CH₂)₅CH₃, —OC₆H₅ and —CH₂C₆H₅.

44. A biodegradable drug conjugate according to claim 43 wherein R¹ is selected from hydroxy, iso-propyloxy and ethylamino.

45. A polymer drug conjugate according to claim 38 wherein the polymer drug conjugate is a polymer of a monomer of formula: 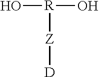
wherein R, Z and D are as hereinbefore defined.

46. A polymer drug conjugate according to claim 45 wherein the polymer is a polyurethane polymer formed with a polyisocyanate and optionally one or more monomers comprising a plurality of active-hydrogen containing groups selected from hydroxy, amine and carboxylic acid.

47. A polymer-drug conjugate obtained by polymerising a drug-monomer conjugate of formula: 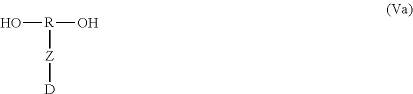
with at least one monomer selected from the group consisting oft polyacid halides, polycarboxylic acids, polycarboxylic acid esters, polycarboxylic anhydrides, polyisocyanates, polyamines, cyclic esters and cyclic carbonates.

48. A polymer-drug conjugate according to claim 47 wherein the drug-monomer conjugate is of formula: 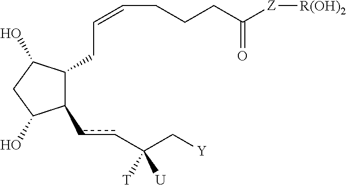
wherein
T and U are each fluoro, or T and U together form oxo, or T is hydroxy and U is hydrogen; and
Z, Y and R are as herein defined.

49. A polymer-drug conjugate according to claim 47 wherein the drug-monomer conjugate is of formula: 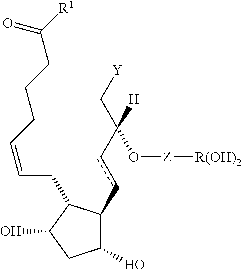
wherein R¹ is OH, C₁ to C₆ alkoxy or C₁ to C₆ alkylamino; and Z, R and Y are as herein defined.

50. A monomer-drug conjugate of formula: 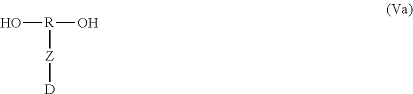
wherein R, Z and D are as defined according to claim 38.

51. A method of preparing a polymer-drug conjugate according to claim 33 comprising polymerising a drug-monomer of formula: 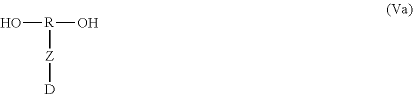
with at least one monomer selected from the group consisting of: polyacid halides, polycarboxylic acids, polycarboxylic acid esters, polycarboxylic anhydrides, polyisocyanates, polyamines, cyclic esters and cyclic carbonates.

52. A method of treatment of glaucoma in a subject suffering glaucoma in one or both eyes, the method comprising administering to an eye afflicted with glaucoma the polymer-drug conjugate according to claim 33.
